(19) United States
(12) Patent Application Publication
(10) Pub. No.: US 2007/0161627 A1
(43) Pub. Date: Jul. 12, 2007

(54) Antibacterial agents

(76) Inventors: William Henry Miller, Collegeville, PA (US); Meagan B. Rouse, Collegeville, PA (US); Mark Andrew Seefeld, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/573,270

(22) PCT Filed: Aug. 9, 2005
(86) PCT No.: PCT/US05/28107

§ 371 Date: Feb. 6, 2007

Related U.S. Application Data
(60) Provisional application No. 60/599,931, filed on Aug. 9, 2004.

(51) Int. Cl. A61K 31/551; A61K 31/5415; A61K 31/5383; A61K 31/496; A61K 31/4745; C07D 498/02; C07D 491/02; C07D 471/02

(52) U.S. Cl. 514/218; 514/224.2; 514/230.5; 514/300; 514/253.04; 540/575; 544/362; 546/123; 544/105; 544/51

(57) ABSTRACT

Naphthalene, quinoline, quinoxaline and naphthyridine derivatives useful in the treatment of bacterial infections in mammals, particularly humans, are disclosed herein.

### FIELD OF THE INVENTION

This invention relates to novel compounds, compositions containing them, their use as antibacterials, and processes for their preparation.

### BACKGROUND OF THE INVENTION

The emergence of pathogens resistant to known antibiotic therapy is becoming a serious global healthcare problem (Chu, et al., (1996) J. Med. Chem., 39: 3853-3874). Thus, there is a need to discover new broad spectrum antibiotics useful in combating multidrug-resistant organisms. Importantly, it has now been discovered that certain compounds have antibacterial activity, and, therefore, may be useful for the treatment of bacterial infections in mammals, particularly in humans.

### SUMMARY OF THE INVENTION

This invention comprises compounds of the formula (I), as described hereinafter, which are useful in the treatment of bacterial infections. This invention is also a pharmaceutical composition comprising a compound according to formula (I) and a pharmaceutically acceptable carrier. This invention is also processes for the preparation of compounds of formula (I), as well as processes for the preparation of intermediates useful in the synthesis of compounds of formula (I). This invention is also a method of treating bacterial infections in mammals, particularly in humans.

### DETAILED DESCRIPTION OF THE INVENTION

In one aspect, this invention provides a compound of formula (I) or a pharmaceutically acceptable salt, solvate or derivative thereof: 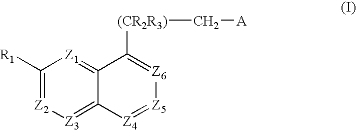
wherein:

Z₁, Z₃, and Z₄ are independently N or CR^{1a};

Z₂, Z₅, and Z₆ are each CR^{1a};

R₁ and R^{1a} are independently at each occurrence hydrogen; cyano; halogen; hydroxy; (C₁₋₆)alkoxy unsubstituted or substituted by (C₁₋₆)alkoxy, hydroxy, amino, piperidyl, guanidino or amidino any of which is unsubstitued or N-substituted by one or two (C₁₋₆)alkyl, acyl, (C₁₋₆)alkylsulphonyl, CONH₂, hydroxy, (C₁₋₆)alkylthio, heterocyclylthio, heterocyclyloxy, arylthio, aryloxy, acylthio, acyloxy or (C₁₋₆)alkylsulphonyloxy; (C₁₋₆)alkyl; (C₁₋₆)alkylthio; trifluoromethyl; trifluoromethoxy; nitro; azido; acyl; acyloxy; acylthio; (C₁₋₆)alkylsulphonyl; (C₁₋₆)alkylsulphoxide; arylsulphonyl; arylsulphoxide; or an amino, piperidyl, guanidino or amidino group unsubstituted or N-substituted by one or two (C₁₋₆)alkyl, acyl or (C₁₋₆)alkylsulphonyl groups; or R₁ and R^{1a} of Z₂ together form ethylenedioxy;

R₂ is hydrogen; halogen; hydroxy; acyloxy; or (C₁₋₆)alkoxy;

R₃ is hydrogen; 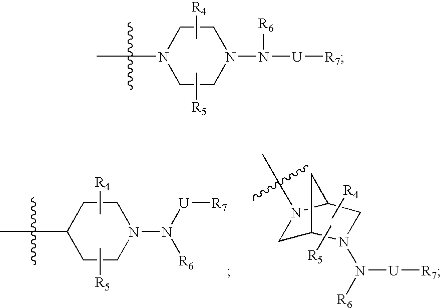
A is

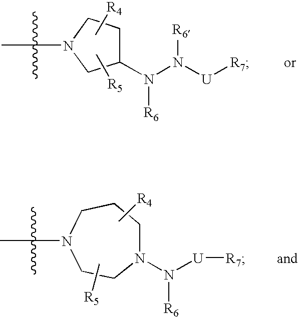

R₄ and R₅ are independently hydrogen; thiol; (C₁₋₆)alkylthio; halogen; trifluoromethyl; azido; (C₁₋₆)alkyl (optionally substituted with hydroxy or (C₁₋₆)alkoxy); (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonyl; aryl; aralkyl; aryl; heterocyclyl; heterocyclylalkyl; hydroxy; NR^{1b}R^{1b′}; (C₁₋₆)alkylsulphonyl; (C₂₋₆)alkenylsulphonyl; or (C₁₋₆)aminosulphonyl wherein the amino group is optionally and independently substituted with hydrogen; (C₁₋₆)alkyl; (C₂₋₆)alkenyl; or aralkyl;

R^{1b} and R^{1b′} are independently at each occurrence hydrogen; (C₁₋₆)alkyl; aralkyl; aryl; heterocyclyl; heterocyclylalkyl; or together with the nitrogen that they are attached form an aziridine, azetidine, pyrrolidine, piperidine or hexamethyleneimine ring (wherein said aziridine, azetidine, pyrrolidine, piperidine or hexamethyleneimine ring are optionally substiuted with from 1 to 3 substituents selected from halogen, hydroxy; cyano; nitro; (C₁₋₆)alkyl; and aryl);

R₆ and R_{6′} are independently hydrogen, trifluoromethyl; (C₁₋₆)alkyl; (C₂₋₆)alkenyl; (C₁₋₆)alkoxycarbonyl; (C₁₋₆)alkylcarbonyl; (C₂₋₆)alkenyloxycarbonyl; aryl; aralkyl; (C₃₋₈)cycloalkyl; heterocyclyl; or heterocyclylalkyl;

U is (C(═O))ₙ or SO₂;

n is 1 or 2;

R₇ is a substituted or unsubstituted bicyclic carbocyclic or heterocyclic ring system (A):

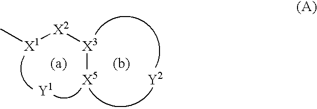

containing up to four heteroatoms in each ring in which at least one of rings (a) and (b) is aromatic;

X¹ is C or N when part of an aromatic ring or CR₈ when part of a non aromatic ring;

X² is N, NR₉, O, S(O)_{n′}, CO, a bond, or CR₈ when part of an aromatic or non-aromatic ring or may in addition be CR₁₀R₁₁ when part of a non aromatic ring;

n′ is independently at each occurrence 0, 1 or 2;

X³ and X⁵ are independently N or C;

Y¹ is a 0 to 4 atom linker group each atom of which is independently selected from N, NR₉, O, S(O)_{n′}, CO and CR₈ when part of an aromatic or non-aromatic ring or may additionally be CR₁₀R₁₁ when part of a non aromatic ring,

Y⁻ is a 2 to 6 atom linker group, each atom of Y² being independently selected from N, NR₉, O, S(O)_{n′}, CO and CR₈ when part of an aromatic or non-aromatic ring or may additionally be CR₁₀R₁₁ when part of a non aromatic ring;

R₈, R₁₀ and R₁₁ are at each occurrence independently selected from: H; (C₁₋₄)alkylthio; halogen; (C₁₋₄)alkyl; (C₂₋₄)alkenyl; hydroxy; hydroxy(C₁₋₄)alkyl; mercapto(C₁₋₄)alkyl; (C₁₋₄)alkoxy; trifluoromethoxy; nitro; cyano; carboxy; amino or aminocarbonyl unsubstituted or substituted by (C₁₋₄)alkyl;

R₉ is at each occurrence independently hydrogen; trifluoromethyl; (C₁₋₄)alkyl unsubstituted or substituted by hydroxy, carboxy, (C₁₋₄)alkoxy, (C₁₋₆)alkylthio, halogen or trifluoromethyl; (C₂₋₄)alkenyl; or aminocarbonyl wherein the amino group is optionally substituted with (C₁₋₄)alkyl;

or a pharmaceutically acceptable salt or solvate thereof.

In some embodiments, this invention describes a compound of formula (I) wherein Z₁ and Z₄ are N and Z₃ is CR^{1a}.

In certain embodiments, this invention describes a compound of formula (I) wherein R₁, is OCH₃.

In some embodiments, this invention describes a compound of formula (I) wherein R^{1a} is at each occurrence independently hydrogen; halogen; or cyano.

In some aspects, this invention describes a compound of formula (I) wherein:

A is

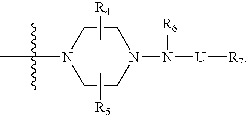

In other aspects, this invention describes a compound of formula (I) wherein:

A is

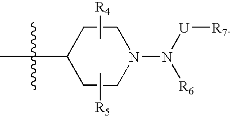

In still yet another aspect, this invention describes a compound of formula (I) wherein:

A is

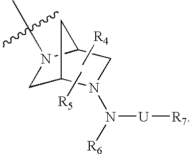

In some embodiments, this invention describes a compound of formuls (I) wherein:

A is

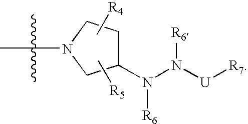

In certain embodiments, this invention describes a compound of formula (I) wherein:

A is

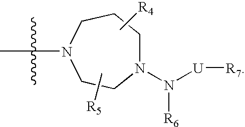

In some embodiments, this invention describes a compound of formula 1, wherein R₇ is Indol-3-yl; 5-Fluoro-indol-2-yl; 4H-Pyrido[3,2-b][1,4]thiazin-3-oxo-6-yl; 4H-Pyrido[3,2-b][1,4]oxazin-3-oxo-6-yl; 2,3-Dihydro-[1,4]dioxino[2,3-c]-pyridin-6-yl; 4H-Benzo[3,2-b][1,4]thiazin-3-oxo-6-yl; 4H-Pyrido[3,2-b][1,4]oxazin-7-chloro-3-oxo-6-yl; or 3,4-Dihydro-2H-benzo[b][1,4]dioxepin-7-yl.

In certain embodiments, this invention describes a compound of formula (I) wherein
A is 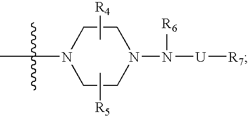
R₁ is OCH₃; Z₁ and Z₄ are N; Z₃ is CR^{1a}; R^{1a} of Z₂, Z₃, and Z₅ is hydrogen; R^{1a} of Z₆ is hydrogen, fluoro, chloro, or cyano; R₂ is hydrogen or hydroxy; R₄ and R₅ are independently hydrogen, hydroxy or (C₁₋₆)alkyl (optionally substituted with hydroxy or (C₁₋₆)alkoxy); and R₆ is hydrogen or (C₁₋₆)alkyl.

In some embodiments, this invention describes a compound of formula (I) wherein
A is 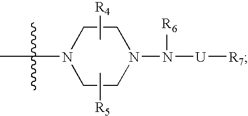
R₁ is OCH₃; Z₁ and Z₄ are N; Z₃ is CR^{1a}; R^{1a} of Z₂, Z₃, and Z₅ is hydrogen; R^{1a} of Z₆ is hydrogen, fluoro, chloro, or cyano; R₂ is hydrogen or hydroxy; R₄ and R₅ are independently hydrogen, hydroxy or (C₁₋₆)alkyl (optionally substituted with hydroxy or (C₁₋₆)alkoxy); R₆ is hydrogen or (C₁₋₆)alkyl; and R₇ is Indol-3-yl; 5-Fluoro-indol-2-yl; 4H-Pyrido[3,2-b][1,4]thiazin-3-oxo-6-yl; 4H-Pyrido[3,2-b][1,4]oxazin-3-oxo-6-yl; 2,3-Dihydro-[1,4]dioxino[2,3-c]-pyridin-6-yl; 4H-Benzo[3,2-b][1,4]thiazin-3-oxo-6-yl; 4H-Pyrido[3,2-b][1,4]oxazin-7-chloro-3-oxo-6-yl; or 3,4-Dihydro-2H-benzo[b][1,4]dioxepin-7-yl.

In certain embodiments, this invention describes a compound of formula (I) wherein
A is 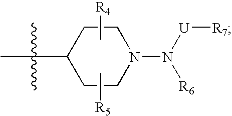
R₁ is OCH₃; Z₁ and Z₄ are N; Z₃ is CR^{1a}; R^{1a} of Z₂, Z₃, and Z₅ is hydrogen; R^{1a} of Z₆ is hydrogen, fluoro, chloro, or cyano; R₂ is hydrogen or hydroxy; R₄ and R₅ are independently hydrogen, hydroxy or (C₁₋₆)alkyl (optionally substituted with hydroxy or (C₁₋₆)alkoxy); and R₆ is hydrogen or (C₁₋₆)alkyl.

In some embodiments, this invention describes a compound of formula (I) wherein
A is 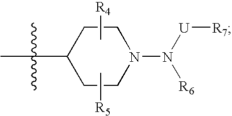
R₁ is OCH₃; Z₁ and Z₄ are N; Z₃ is CR^{1a}; R^{1a} of Z₂, Z₃, and Z₅ is hydrogen; R^{1a} of Z₆ is hydrogen, fluoro, chloro, or cyano; R₂ is hydrogen or hydroxy; R₄ and R₅ are independently hydrogen, hydroxy or (C₁₋₆)alkyl (optionally substituted with hydroxy or (C₁₋₆)alkoxy); R₆ is hydrogen or (C₁₋₆)alkyl; and R₇ is Indol-3-yl; 5-Fluoro-indol-2-yl; 4H-Pyrido[3,2-b][1,4]thiazin-3-oxo-6-yl; 4H-Pyrido[3,2-b][1,4]oxazin-3-oxo-6-yl; 2,3-Dihydro-[1,4]dioxino[2,3-c]-pyridin-6-yl; 4H-Benzo[3,2-b][1,4]thiazin-3-oxo-6-yl; 4H-Pyrido[3,2-b][1,4]oxazin-7-chloro-3-oxo-6-yl; or 3,4-Dihydro-2H-benzo[b][1,4]dioxepin-7-yl.

In certain embodiments, this invention describes a compound of formula (I) wherein
A is 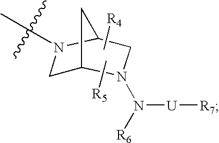
R₁ is OCH₃; Z₁ and Z₄ are N; Z₃ is CR^{1a}; R^{1a} of Z₂, Z₃, and Z₅ is hydrogen; R^{1a} of Z₆ is hydrogen, fluoro, chloro, or cyano; R₂ is hydrogen or hydroxy; R₄ and R₅ are independently hydrogen, hydroxy or (C₁₋₆)alkyl (optionally substituted with hydroxy or (C₁₋₆)alkoxy); and R₆ is hydrogen or (C₁₋₆)alkyl.

In some embodiments, this invention describes a compound of formula (I) wherein
A is 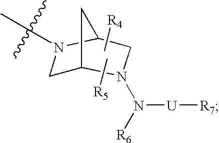
R₁ is OCH₃; Z₁ and Z₄ are N; Z₃ is CR^{1a}; R^{1a} of Z₂, Z₃, and Z₅ is hydrogen; R^{1a} of Z₆ is hydrogen, fluoro, chloro, or cyano; R₂ is hydrogen or hydroxy; R₄ and R₅ are independently hydrogen, hydroxy or (C₁₋₆)alkyl (optionally substituted with hydroxy or (C₁₋₆)alkoxy); R₆ is hydrogen or (C₁₋₆)alkyl; and R₇ is Indol-3-yl; 5-Fluoro-indol-2-yl; 4H-Pyrido[3,2-b][1,4]thiazin-3-oxo-6-yl; 4H-Pyrido[3,2-b][1,4]oxazin-3-oxo-6-yl; 2,3-Dihydro-[1,4]dioxino[2,3-c]-pyridin-6-yl; 4H-Benzo[3,2-b][1,4]thiazin-3-oxo-6-yl; 4H-Pyrido[3,2-b][1,4]oxazin-7-chloro-3-oxo-6-yl; or 3,4-Dihydro-2H-benzo[b][1,4]dioxepin-7-yl.

In certain embodiments, this invention describes a compound of formula (I) wherein
A is 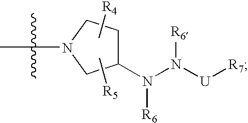
R₁ is OCH₃; Z₁ and Z₄ are N; Z₃ is CR^{1a}; R^{1a} of Z₂, Z₃, and Z₅ is hydrogen; R^{1a} of Z₆ is hydrogen, fluoro, chloro, or cyano; R₂ is hydrogen or hydroxy; R₄ and R₅ are independently hydrogen, hydroxy or (C₁₋₆)alkyl (optionally substituted with hydroxy or (C₁₋₆)alkoxy); and R₆ and R_{6′} are independently hydrogen or (C₁₋₆)alkyl.

In some embodiments, this invention describes a compound of formula (I) wherein
A is 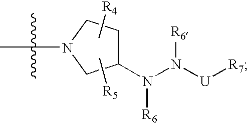
R₁ is OCH₃; Z₁ and Z₄ are N; Z₃ is CR^{1a}; R^{1a} of Z₂, Z₃, and Z₅ is hydrogen; R^{1a} of Z₆ is hydrogen, fluoro, chloro, or cyano; R₂ is hydrogen or hydroxy; R₄ and R₅ are independently hydrogen, hydroxy or (C₁₋₆)alkyl (optionally substituted with hydroxy or (C₁₋₆)alkoxy); R₆ and R_{6′} are independently hydrogen or (C₁₋₆)alkyl; and R₇ is Indol-3-yl; 5-Fluoro-indol-2-yl; 4H-Pyrido[3,2-b][1,4]thiazin-3-oxo-6-yl; 4H-Pyrido[3,2-b][1,4]oxazin-3-oxo-6-yl; 2,3-Dihydro-[1,4]dioxino[2,3-c]-pyridin-6-yl; 4H-Benzo[3,2-b][1,4]thiazin-3-oxo-6-yl; 4H-Pyrido[3,2-b][1,4]oxazin-7-chloro-3-oxo-6-yl; or 3,4-Dihydro-2H-benzo[b][1,4]dioxepin-7-yl.

In certain embodiments, this invention describes a compound of formula (I) wherein

A is 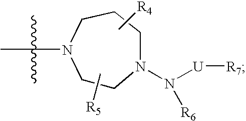
R₁ is OCH₃; Z₁ and Z₄ are N; Z₃ is CR^{1a}; R^{1a} of Z₂, Z₃, and Z₅ is hydrogen; R^{1a} of Z₆ is hydrogen, fluoro, chloro, or cyano; R₂ is hydrogen or hydroxy; R₄ and R₅ are independently hydrogen, hydroxy or (C₁₋₆)alkyl (optionally substituted with hydroxy or (C₁₋₆)alkoxy); and R₆ is hydrogen or (C₁₋₆)alkyl.

In some embodiments, this invention describes a compound of formula (I) wherein
A is 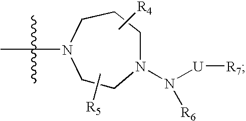
R₁ is OCH₃; Z₁ and Z₄ are N; Z₃ is CR^{1a}; R^{1a} of Z₂, Z₃, and Z₅ is hydrogen; R^{1a} of Z₆ is hydrogen, fluoro, chloro, or cyano; R₂ is hydrogen or hydroxy; R₄ and R₅ are independently hydrogen, hydroxy or (C₁₋₆)alkyl (optionally substituted with hydroxy or (C₁₋₆)alkoxy); R₆ is hydrogen or (C₁₋₆)alkyl; and R₇ is Indol-3-yl; 5-Fluoro-indol-2-yl; 4H-Pyrido[3,2-b][1,4]thiazin-3-oxo-6-yl; 4H-Pyrido[3,2-b][1,4]oxazin-3-oxo-6-yl; 2,3-Dihydro-[1,4]dioxino[2,3-c]-pyridin-6-yl; 4H-Benzo[3,2-b][1,4]thiazin-3-oxo-6-yl 4H-Pyrido[3,2-b][1,4]oxazin-7-chloro-3-oxo-6-yl; or 3,4-Dihydro-2H-benzo[b][1,4]dioxepin-7-yl.

In certain embodiments, this invention describes a compound of formula (I) wherein the compound is N-(4-{2-[6-(methyloxy)-1,5-naphthyridin-4-yl]ethyl}-1-piperazinyl)-3-oxo-3,4-dihydro-2H-pyrido[3,2-b][1,4]thiazine-6-carboxamide; N-(4-{2-[6-(methyloxy)-1,5-naphthyridin-4-yl]ethyl}-1-piperazinyl)-3-oxo-3,4-dihydro-2H-pyrido[3,2-b][1,4]thiazine-6-sulfonamide; N-(4-{2-[3-fluoro-6-(methyloxy)-1,5-naphthyridin-4-yl]ethyl}-1-piperazinyl)-3-oxo-3,4-dihydro-2H-pyrido[3,2-b][1,4]thiazine-6-carboxamide; N-(4-{(2S)-2-hydroxy-2-[6-(methyloxy)-1,5-naphthyridin-4-yl]ethyl}-1-piperazinyl)-3-oxo-3,4-dihydro-2H-pyrido[3,2-b][1,4]thiazine-6-carboxamide; N-(4-{2-[3-chloro-6-(methyloxy)-1,5-naphthyridin-4-yl]ethyl}-1-piperazinyl)-3-oxo-3,4-dihydro-2H-pyrido[3,2-b][1,4]thiazine-6-carboxamide; N-(4-{2-[3-chloro-6-(methyloxy)-1,5-naphthyridin-4-yl]ethyl}-1-piperazinyl)-3-oxo-3,4-dihydro-2H-pyrido[3,2-b][1,4]oxazine-6-carboxamide; N-methyl-N-(4-{2-[6-(methyloxy)-1,5-naphthyridin-4-yl]ethyl}-1-piperazinyl)-3-oxo-3,4-dihydro-2H-pyrido[3,2-b][1,4]thiazine-6-carboxamide; N-4-{2-[3-fluoro-6-(methyloxy)-1,5-naphthyridin-4-yl]ethyl}-1-piperazinyl)-3-oxo-3,4-dihydro-2H-pyrido[3,2-b][1,4]oxazine-6-carboxamide; N-(4-{2-[3-fluoro-6-(methyloxy)-1,5-naphthyridin-4-yl]ethyl}-1-piperazinyl)-2,3-dihydro[1,4]dioxino[2,3-c]pyridine-7-carboxamide; 7-chloro-N-(4-{2-[3-fluoro-6-(methyloxy)-1,5-naphthyridin-4-yl]ethyl}-1-piperazinyl)-3-oxo-3,4-dihydro-2H-pyrido[3,2-b][1,4]oxazine-6-carboxamide; N-((1S,4S)-5-{2-[3-fluoro-6-(methyloxy)-1,5-naphthyridin-4-yl]ethyl}-2,5diazabicyclo[2.2.1]hept-2-yl)-3-oxo-3,4-dihydro-2H-pyrido[3,2-b][1,4]thiazine-6-carboxamide; N-methyl-N-(1-{2-[6-(methyloxy)-1,5-naphthyridin-4-yl]ethyl}-3-pyrrolidinyl)-3-oxo-3,4-dihydro-2H-pyrido[3,2-b][1,4]thiazine-6-carboxamide; N-(4-hydroxy-4-{2-[6-(methyloxy)-1,5-naphthyridin-4-yl]ethyl}-1-piperidinyl)-3-oxo-3,4-dihydro-2H-pyrido[3,2-b][1,4]thiazine-6-carboxamide; N-(4-{2-[3-fluoro-6-(methyloxy)-1,5-naphthyridin-4-yl]ethyl}-4-hydroxy-1-piperidinyl)-3-oxo-3,4-dihydro-2H-pyrido[3,2-b][1,4]thiazine-6-carboxamide; 5-fluoro-N-(4-{2-[3-fluoro-6-(methyloxy)-1,5-naphthyridin-4-yl]ethyl}-1-piperazilnyl)-1H-indole-2-carboxamide; N-(4-{2-[3-fluoro-6-(methyloxy)-1,5-naphthyridin-4-yl]ethyl}-1-piperazinyl)-3,4-dihydro-2H-1,5-benzodioxepin-7-carboxamide; N-(4-{2-[3-fluoro-6-(methyloxy)-1,5-naphthyridin-4-yl]ethyl}-1-piperazinyl)-2-(1H-indol-3-yl)-2-oxoacetamide; N-((2R,5S)-4-{2-[3-fluoro-6-(methyloxy)-1,5-naphthyridin-4-yl]ethyl}-2,5-dimethyl-1-piperazinyl)-3-oxo-3,4-dihydro-2H-pyrido[3,2-b][1,4]thiazine-6-carboxamide; N-(4-{2-[3-fluoro-6-(methyloxy)-1,5-naphthyridin-4-yl]ethyl}-2-methyl-1-piperazinyl)-3-oxo-3,4-dihydro-2 H-pyrido[3,2-b][1,4]thiazine-6-carboxamide; N-(4-{2-[3-fluoro-6-(methyloxy)-1,5-naphthyridin-4-yl]ethyl}hexahydro-1H-1,4-diazepin-1-yl)-3-oxo-3,4-dihydro-2H-pyrido[3,2-b][1,4]thiazine-6-carboxamide; N-(4-{(2S)-[3-fluoro-6-(methyloxy)-1,5-naphthyridin-4-yl]-2-hydroxyethyl}-1-piperazinyl)-3-oxo-3,4-dihydro-2H-pyrido[3,2-b][1,4]thiazine-6-carboxamide; N-(4-{(2R)-[3-fluoro-6-(methyloxy)-1,5-naphthyridin-4-yl]-2-hydroxyethyl}-1-piperazinyl)-3-oxo-3,4-dihydro-2H-pyrido[3,2-b][1,4]thiazine-6-carboxamide; N-(4-{2-[3-cyano-6-(methyloxy)-1,5-naphthyridin-4-yl]ethyl}-1-piperazinyl)-3-oxo-3,4-dihydro-2H-pyrido[3,2-b][1,4]thiazine-6-carboxamide; N-[4-{2-[3-fluoro-6-(methyloxy)-1,5-naphthyridin-4-yl]ethyl}-2-(hydroxymethyl)-1-piperazinyl]-3-oxo-3,4-dihydro-2H-pyrido[3,2-b][1,4]thiazine-6-carboxamide; or N-{4-{2-[3-fluoro-6-(methyloxy)-1,5-naphthyridin-4-yl]ethyl}-2-[(methyloxy)methyl]-1-piperazinyl}-3-oxo-3,4-dihydro-2H-pyrido[3,2-b][1,4]thiazine-6-carboxamide; or a pharmaceutically acceptable salt or solvate thereof.

In some embodiments, this invention describes a compound of formula (I), which process comprises:

(a) reaction of a compound of formula (II) with a compound of formula (Ill) to form a compound of formula (I);
wherein:

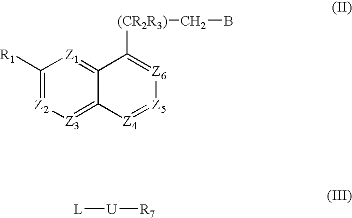

Z₁, R₁, Z₂, Z₃, Z₄, Z₅, Z₆, R₂, R₃, R₄, R₅, R₆, R_{6′}, R₇ and U are as defined in claim 1; 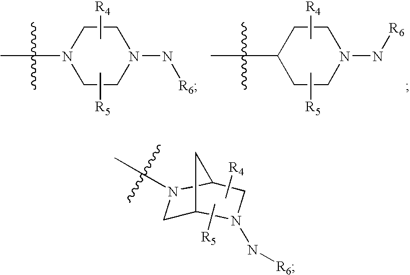
B is

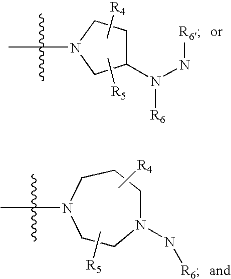

L is a leaving group.

In certain embodiments, this invention describes a pharmaceutical composition comprising a compound of formula I or any one of the embodiments described herein, and a pharmaceutically acceptable carrier.

In some embodiments, this invention describes a method of treating bacterial infections which comprises administering to a mammal in need thereof an effective amount of a compound of formula I or any of its embodiments described herein.

In some embodiments, this invention describes compounds of formula I wherein the (a) and (b) rings of R₁₁ are both aromatic as demonstrated by the following non-limiting examples: 1H-pyrrolo[2,3-b]-pyridin-2-yl, 1H-pyrrolo[3,2-b]-pyridin-2-yl, 3H-imidazo[4,5-b]-pyrid-2-yl, 3H-quinazolin-4-one-2-yl, benzimidazol-2-yl, benzo[1,2,3]-thiadiazol-5-yl, benzo[1,2,5]-oxadiazol-5-yl, benzofur-2-yl, benzothiazol-2-yl, benzo[b]thiophen-2-yl, benzoxazol-2-yl, chromen-4-one-3-yl, imidazo[1,2-a]pyridin-2-yl, imidazo-[1,2-a]-pyrimidin-2-yl, indol-2-yl, indol-6-yl, isoquinolin-3-yl, [1,8]-naphthyridine-3-yl, oxazolo[4,5-b]-pyridin-2-yl, quinolin-2-yl, quinolin-3-yl, quinoxalin-2-yl, indan-2-yl, naphthalen-2-yl, 1,3-dioxo-isoindol-2yl, benzimidazol-2-yl, benzothiophen-2-yl, 1H-benzotriazol-5-yl, 1H-indol-5-yl, 3H-benzooxazol-2-one-6-yl, 3H-benzooxazol-2-thione-6-yl, 3H-benzothiazol-2-one-5-yl, 3H-quinazolin-4-one-2-yl, 3H-quinazolin-4-one-6-yl, 4-oxo-4H-pyrido[1,2-a]pyrimidin-3-yl, benzo[1,2,3]thiadiazol-6-yl, benzo[1,2,5]thiadiazol-5-yl, benzo[1,4]oxazin-2-one-3-yl, benzothiazol-5-yl, benzothiazol-6-yl, cinnolin-3-yl, imidazo[1,2-a]pyridazin-2-yl, imidazo[1,2-b]pyridazin-2-yl, pyrazolo[1,5-a]pyrazin-2-yl, pyrazolo[1,5-a]pyridin-2-yl, pyrazolo[1,5-a]pyrimidin-6-yl, pyrazolo[5,1-c][1,2,4]triazin-3-yl, pyrido[1,2-a]pyrimdin-4-one-2-yl, pyrido[1,2-a]pyrimidin-4-one-3-yl, quinazolin-2-yl, quinoxalin-6-yl, thiazolo[3,2-a]pyrimidin-5-one-7-yl, thiazolo[5,4-b]pyridin-2-yl, thieno[3,2-b]pyridin-6-yl, thiazolo[5,4-b]pyridin-6-yl, 4-oxo-4H-pyrido[1,2-a]pyrimidin-2-yl, 1-oxo-1,2-dihydro-isoquinolin-3-yl, thiazolo[4,5-b]pyridin-5-yl, [1,2,3]thiadiazolo[5,4-b]pyridin-6-yl, 2H-isoquinolin-1-one-3-yl.

In yet other embodiments, R₁₁ is defined by a non-aromatic (a) ring and aromatic (b) ring as illustrated by the following non-limiting examples:_(2S)-2,3-dihydro-1H-indol-2-yl, (2S)-2,3-dihydro-benzo[1,4]dioxine-2-yl, 3-(R,S)-3,4-dihydro-2H-benzo[1,4]thiazin-3-yl, 3-(R)-2,3-dihydro-[1,4]dioxino[2,3-b]pyridin-3-yl, 3-(S)-2,3-dihydro-[1,4]dioxino[2,3-b]pyridin-3-yl, 2,3-dihydro-benzo[1,4]dioxan-2-yl, 3-substituted-3H-quinazolin-4-one-2-yl, 2,3-dihydro-benzo[1,4]dioxan-2-yl, 1-oxo-1,3,4,5-tetrahydrobenzo[c]azepin-2-yl. In still other embodiments, R₁₁ is defined by an aromatic (a) ring and a non aromatic (b) ring as illustrated by the following non-limiting examples: 1,1,3-trioxo-1,2,3,4-tetrahydro-1 I⁶-benzo[1,4]thiazin-6-yl, benzo[1,3]dioxol-5-yl, 2,3-dihydro-benzo[1,4]dioxin-6-yl, 2-oxo-2,3-dihydro-benzooxazol-6-yl, 4H-benzo[1,4]oxazin-3-one-6-yl (3-oxo-3,4-dihydro-2H-benzo[1,4]oxazin-6-yl), 4H-benzo[1,4]thiazin-3-one-6-yl(3-oxo-3,4-dihydro-2H-benzo[1,4]thiazin-6-yl), 4H-benzo[1,4]oxazin-3-one-7-yl, 4-oxo-2,3,4,5-tetrahydro-benzo[b][1,4]thiazepine-7-yl, 5-oxo-2,3-dihydro-5H-thiazolo[3,2-a]pyrimidin-6-yl, benzo[1,3]dioxol-5-yl, 2-oxo-2,3-dihydro-1H-pyrido[2,3-b][1,4]thiazin-7-yl, 2-oxo-2,3-dihydro-1H-pyrido[3,4-b][1,4]thiazin-7-yl, 3-oxo-3,4-dihydro-2H-pyrido[3,2-b][1,4]thiazin-6-yl, 2,3-dihydro-[1,4]dioxino[2,3-b]pyridin-6-yl, 2,3-dihydro-[1,4]dioxino[2,3-c]pyridin-7-yl, 2,3-dihydro-[1,4]dioxino[2,3-b]pyridin-7-yl, 6,7-dihydro-[1,4]dioxino[2,3-d]pyrimidin-2-yl, 3-oxo-3,4-dihydro-2H-pyrido[3,2-b][1,4]oxazin-6-yl, 2-oxo-2,3-dihydro-1H-pyrido[3,4-b][1,4]oxazin-7-yl, 2-oxo-2,3-dihydro-1H-pyrido[2,3-b][1,4]oxazin-7-yl, 6-oxo-6,7-dihydro-5H-8-thia-1,2,5-triaza-naphthalen-3-yl, 3,4-dihydro-2H-benzo[1,4]oxazin-6-yl, 3-substituted-3H-benzooxazol-2-one-6-yl, 3-substituted-3H-benzooxazole-2-thione-6-yl, 3-substituted-3H-benzothiazol-2-one-6-yl, 2,3-dihydro-1H-pyrido[2,3-b][1,4]thiazin-7-yl, 3,4-dihydro-2H-benzo[1,4]thiazin-6-yl, 3,4-dihydro-1H-quinolin-2-one-7-yl, 3,4-dihydro-1H-quinoxalin-2-one-7-yl, 6,7-dihydro-4H-pyrazolo[1,5-a]pyrimidin-5-one-2-yl, 5,6,7,8-tetrahydro-[1,8]naphthyridin-2-yl, 2-oxo-3,4-dihydro-1H-[1,8]naphthyridin-6-yl, 3,4-dihydro-2H-pyrido[3,2-b][1,4]thiazin-6-yl.

Unless otherwise defined, the term “alkyl” when used alone or when forming part of other groups (such as the ‘alkoxy’ group) includes substituted or unsubstituted, straight or branched chain alkyl groups containing the specified range of carbon atoms. For example, the term “(C₁₋₆)alkyl” include methyl; ethyl, propyl, butyl, iso-propyl, sec-butyl, tert-butyl, iso-pentyl, and the like.

The term “alkenyl” means a substituted or unsubstituted alkyl group of the specified range of carbon atoms, wherein one carbon-carbon single bond is replaced by a carbon-carbon double bond. For example, the term “(C₂₋₆)alkenyl” include ethylene, 1-propene, 2-propene, 1-butene, 2-butene, and isobutene, and the like. Both cis and trans isomers are included.

The term “cycloalkyl” refers to subsituted or unsubstituted carbocyclic system of the specifed range of carbon atoms, which may contain up to two unsaturated carbon-carbon bonds. For example, the term “(C₃₋₇)cycloalkyl” include cyclopropyl, cyclobutyl, cyclopentyl, cyclopentenyl, cyclohexyl, cyclohexenyl, and cycloheptyl.

The term “alkoxy” refers to an O-alkyl radical where the alkyl group contains the specified range of carbon atoms and is as defined herein.

The term “acyl” refers to a C(═O)alkyl or a C(═O)aryl radical. In some embodiments, the alkyl group contains 13 or less carbons; in some embodiments 10 or less carbon atoms; in some embodiments 6 or less carbon atoms; and is as otherwise defined. Aryl is as defined herein.

The term “alkylcarbonyl” refers to a (C₁₋₆)alkyl(C═O)(C₁₋₆)alkyl group wherein alkyl is as otherwise defined herein.

The term “alkylsulphonyl” refers to a SO₂alkyl radical wherein the alkyl group contains the specified range of carbon atoms and is as defined herein.

The term “alkylthio” refers to a Salkyl wherein the alkyl group contains the specified range of carbon atoms and is as defined herein.

The term “aminosulphonyl” refers to a SO₂N radical wherein the nitrogen is substituted as specified.

The term “aminocarbonyl” refers to a carboxamide radical wherein the nitrogen of the amide is substituted as defined.

The term “heterocyclylthio” refers to a S-heterocyclyl radical wherein the heterocyclyl moiety is as defined herein.

The term “heterocyclyloxy” refers to an O-heterocyclyl radical wherein heterocyclyl is as defined herein.

The term “arylthio” refers to an S-aryl radical wherein aryl is as defined herein.

The term “aryloxy” refers to an O-aryl radical wherein aryl is as defined herein.

The term “acylthio” refers to a S-acyl radical wherein acyl is as defined herein.

The term “acyloxy” refers to an O-acyl radical wherein acyl is as defined herein.

The term “alkoxycarbonyl” refers to a CO₂alkyl radical wherein the alkyl group contains the specified range of carbon atoms and is as defined herein.

The term “alkenyloxycarbonyl” refers to a CO₂alkyl radical wherein the alkenyl group contains the specified range of carbon atoms and is as defined herein.

The term “alkylsulphonyloxy” refers to an O—SO₂alkyl radical wherein the alkyl group contains the specified range of carbon atoms and is as defined herein.

The term “arylsulphonyl” refers to a SO₂aryl radical wherein aryl is as herein defined.

The term “arylsulphoxide” refers to a SOaryl radical wherein aryl is as defined herein.

Unless otherwise defined, suitable substituents for any alkyl, alkoxy, alkenyl, and cycloalkyl groups includes up to three substituents selected from the group consisting of hydroxy, halogen, nitro, cyano, carboxy, amino, amidino, sulphonamido, unsubstituted (C₁₋₃)alkoxy, trifluromethyl, and acyloxy.

Halo or halogen includes fluoro, chloro, bromo and iodo.

The term “haloalkyl” refers to an alkyl radical containing the specified range of carbon atoms and is as otherwise defined herein, which is further substituted with 1-3 halogen atoms.

The term “haloalkoxy” refers to an alkoxy radical of the specified range and as defined herein, which is further substituted with 1-3 halogen atoms.

The term “hydroxyalkyl” refers to an alkyl group as defined herein, further substituted with a hydroxy group.

Unless otherwise defined, the term “heterocyclic” or “heterocyclyl” as used herein includes optionally substituted aromatic and non-aromatic, single and fused, mono- or bicyclic rings suitably containing up to four hetero-atoms in each ring selected from oxygen, nitrogen and sulphur, which rings may be unsubstituted or C-substituted by, for example, up to three groups selected from (C₁₋₄)alkylthio; halo; (C₁₋₄)haloalkoxy; (C₁₋₄)haloalkyl; (C₁₋₄)alkyl; (C₂₋₄)alkenyl; hydroxy; hydroxy, (C₁₋₄)alkyl; (C₁₋₄)thioalkyl; (C₁₋₄)alkoxy; nitro; cyano, carboxy; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl.

Each heterocyclic ring suitably has from 3 to 7, preferably 5 or 6, ring atoms. A fused heterocyclic ring system may include carbocyclic rings and need include only one heterocyclic ring.

Compounds within the invention containing a heterocyclyl group may occur in two or more tautometric forms depending on the nature of the heterocyclyl group; all such tautomeric forms are included within the scope of the invention.

Where an amino group forms part of a single or fused non-aromatic heterocyclic ring as defined above suitable optional substituents in such substituted amino groups include hydrogen; trifluoromethyl; (C₁₋₄)alkyl optionally substituted by hydroxy, (C₁₋₄)alkoxy, (C₁₋₄)alkylthio, halo or trifluoromethyl; and (C₂₋₄)alkenyl.

The term “heterocyclylalkyl” refers to a (C₁₋₆)alkyl radical which bears as a substituent a heterocyclyl group, wherein heterocyclyl and alkyl are as herein defined. The heterocyclyl group maybe joined to a primary, secondary or tertiary carbon of the (C₁₋₆)alkyl chain.

When used herein the term “aryl”, includes optionally substituted phenyl and naphthyl.

Aryl groups may be optionally substituted with up to five, preferably up to three, groups selected from (C₁₋₄)alkylthio; halo; (C₁₋₄)haloalkoxy; (C₁₋₄)haloalkyl; (C₁₋₄)alkyl; (C₂₋₄)alkenyl; hydroxy; (C₁₋₄)hydroxyalkyl; (C₁₋₄)alkylthio; (C₁₋₄)alkoxy; nitro; cyano; carboxy; amino or aminocarbonyl optionally substituted by (C₁₋₄)alkyl; (C₁₋₄)alkylsulphonyl; (C₂₋₄)alkenylsulphonyl.

The term “aralkyl” refers to a (C₁₋₆)alkyl radical which bears as a substituent an aryl group, wherein aryl and alkyl are as herein defined. The aryl group maybe joined to a primary, secondary or tertiary carbon of the (C₁₋₆)alkyl chain.

This invention also contemplates that some of its structural embodiments maybe present as a solvate. Solvates maybe produced from crstallization from a given solvent or mixture of solvents, inorganic or organic. Solvates may also produced upon contact or exposure to solvent vapors, such as water. This invention includes within its scope stoichiometric and non-stoichiometric solvates including hydrates.

Furthermore, it will be understood that phrases such as “a compound of Formula I or a pharmaceutically acceptable salt, solvate or derivative thereof” are intended to encompass the compound of Formula I, a derivative of formula (I), a pharmaceutically acceptable salt of the compound of formula (I), a solvate of formula (I), or any pharmaceutically acceptable combination of these. Thus by way of non-limiting example used here for illustrative purpose, “a compound of Formula I or a pharmaceutically acceptable salt or solvate thereof” may include a pharmaceutically acceptable salt of a compound of formula (I) that is further present as a solvate.

Since the compounds of formula (I) are intended for use in pharmaceutical compositions it will readily be understood that they are each provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the formula (I) or pharmaceutically acceptable derivative thereof.

Pharmaceutically acceptable salts of the above-mentioned compounds of formula (I) include the free base form or their acid addition or quaternary ammonium salts, for example their salts with mineral acids e.g. hydrochloric, hydrobromic, sulphuric, nitric or phosphoric acids, or organic acids, e.g. acetic, fumaric, succinic, maleic, citric, benzoic, p-toluenesulphonic, methanesulphonic, naphthalenesulphonic acid or tartaric acids. Compounds of formula (I) may also be prepared as the N-oxide. Compounds of formula (I) having a free carboxy group may also be prepared as an in vivo hydrolysable ester. The invention extends to all such derivatives. One of skill in the art will recognize that where compounds of the invention contain multiple basic sites, a compound of the invention maybe present as a salt complexed with more than one equivalent of a corresponding acid or mixture of acids.

Pharmaceutically acceptable derivatives refers to compounds of formula (I) that have been covalently modifed with a group that undergoes at least some in vivo cleavage to a compound of formula (I).

Examples of suitable pharmaceutically acceptable in vivo hydrolysable ester-forming groups include those forming esters which break down readily in the human body to leave the parent acid or its salt.

Suitable groups of this type include those of part formulae (i), (ii), (iii), (iv) and (v):

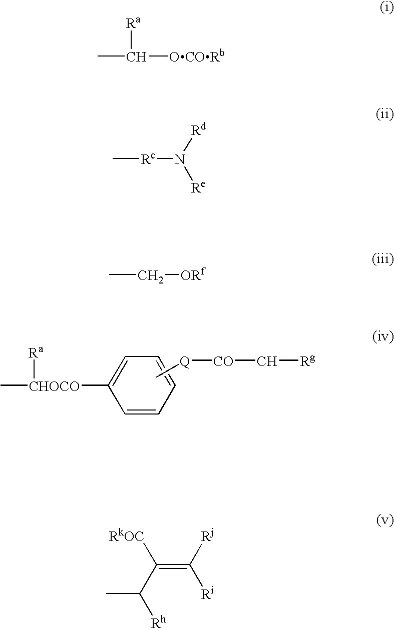

wherein R^{a} is hydrogen, (C₁₋₆) alkyl, (C₃₋₇) cycloalkyl, methyl, or phenyl, R^{b} is (C₁₋₆) alkyl, (C₁₋₆)alkoxy, phenyl, benzyl, (C₃₋₇)cycloalkyl, (C₃₋₇)cycloalkyloxy, (C₁₋₆)alkyl(C₃₋₇) cycloalkyl, 1-amino(C₁₋₆)alkyl, or

1-(C₁₋₆alkyl)amino(C₁₋₆) alkyl; or R^{a} and R^{b} together form a 1,2-phenylene group optionally substituted by one or two methoxy groups; R^{c} represents (C₁₋₆)alkylene optionally substituted with a methyl or ethyl group and R^{d} and R^{e} independently represent (C₁₋₆) alkyl; R^{f} represents (C₁₋₆) alkyl; R^{g} represents hydrogen or phenyl optionally substituted by up to three groups selected from halogen, (C₁₋₆) alkyl, or (C₁₋₆) alkoxy; Q is oxygen or NH; R^{h} is hydrogen or

(C₁₋₆) alkyl; Rⁱ is hydrogen, (C₁₋₆) alkyl optionally substituted by halogen, (C₂₋₆) alkenyl, (C₁₋₆)alkoxycarbonyl, aryl or heteroaryl; or R^{h} and Rⁱ together form (C₁₋₆) alkylene; R^{j} represents hydrogen, (C₁₋₆) alkyl or (C₁₋₆)alkoxycarbonyl; and R^{k} represents (C₁₋₈)alkyl, (C₁₋₈)alkoxy, (C₁₋₆)alkoxy(C₁₋₆)alkoxy or aryl.

Examples of suitable in vivo hydrolysable ester groups include, for example, acyloxy(C₁₋₆)alkyl groups such as acetoxymethyl, pivaloyloxymethyl, acetoxyethyl, pivaloyloxyethyl, 1-(cyclohexylcarbonyloxy)prop-1-yl, and (1-aminoethyl)carbonyloxymethyl; (C₁₋₆)alkoxycarbonyloxy(C₁₋₆)alkyl groups, such as ethoxycarbonyloxymethyl, ethoxycarbonyloxyethyl and propoxycarbonyloxyethyl; di(C₁₋₆)alkylamino(C₁₋₆)alkyl especially di(C₁₋₄)alkylamino(C₁₋₄)alkyl groups such as dimethylaminomethyl, dimethylaminoethyl, diethylaminomethyl or diethylaminoethyl; 2-(C₁₋₆)alkoxycarbonyl)-2-(C₂₋₆)alkenyl groups such as 2-(isobutoxycarbonyl)pent-2-enyl and 2-(ethoxycarbonyl)but-2-enyl; lactone groups such as phthalidyl and dimethoxyphthalidyl.

A further suitable pharmaceutically acceptable in vivo hydrolysable ester-forming group is that of the formula:

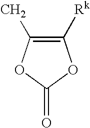

wherein R^{k} is hydrogen, C₁₋₆alkyl or phenyl.

R^{k} is preferably hydrogen.

Compounds of formula (I) may also be prepared as the corresponding N-oxides.

Certain of the compounds of formula (I) may exist in the form of optical isomers, e.g. diastereoisomers and mixtures of isomers in all ratios, e.g. racemic mixtures. The invention includes all such form, including pure isomeric forms. The different isomeric forms may be separated or resolved one from the other by conventional methods, or any given isomer may be obtained by conventional synthetic methods or by stereospecific or asymmetric syntheses.

One of skill in the readily appreciates that optimization for a given reaction may require some routine variation in reaction parmeters such as reaction time, temperature, energy source, pressure, light, pressure, solvent or solvents used, co-reagents, catalysts, and the like.

Protective groups wherever found herein maybe designated by their specific formula or alternatively, maybe referred to generically by P or Pₙ (wherein n is an integer). It is to be appreciated that where generic descriptors are used, that such descriptors are at each occurrence independent from each other. Thus, a compound with more than one of the same generic descriptors (e.g. P) does not indicate that each P is the same protective group, they maybe the same or different, so long as the group is suitable to the chemistry being employed. Where protection or deprotection is generically referred to, one of ordinary skill in the art will understand this to mean that suitable conditions are employed that will allow for the removal of the protecting group to be removed while minimizing reaction at other positions of the molecule, unless otherwise indicated. Many protective groups and protective group strategies are known to those of skill in the art in maybe found in numerous references including, Greene, et al. “Protective Groups in Organic Synthesis” (Published by Wiley-Interscience), which is herein incorporated by reference in its entirety.

Leaving groups wherever found herein maybe designated by a specific chemical formula, or alternatively, maybe generically referred to as L or Ln (wherein n is an integer). It is to be appreciated that where a generic descriptor is used, that such descriptors are at each occurrence independent from each other. Leaving groups can be single atoms such as Cl, Br, or I, or maybe a group such as OSO₂CH₃, OC(═O)CH₃, O(C═O)CF₃, OSO₂CF₃, and the like. Leaving groups may be formed during the course of a reaction and thus a compound containing a leaving group may not always be an isolated material but rather as a reactive intermediate. By way of non-limiting example, a carboxylic acid maybe reacted with a coupling reagent such as DCC, CDI, EDCI, isobutyl chloroformate, etc, and the corresponding reative intermediate thus formed is further reacted with the nucleophilic coupling partner. In such cases, one of skill in the art appreciates that the activation step maybe performed before the introduction of the nucleophilic coupling partner, or in some cases, even in the presence of the nucleophilic coupling partner (depending upon the identity of the particular activating agent, carboxylic acid and nuclephilic coupling partner used). One skilled in the art readily ascertains that leaving groups generally refer to atoms or groups which can be eliminated, substituted or otherwise dissociate during the course of the reaction.

The antibacterial compounds according to the invention may be formulated for administration in any convenient way for use in human or veterinary medicine, by analogy with other antibacterials.

The pharmaceutical compositions of the invention include those in a form adapted for oral, topical or parenteral use and may be used for the treatment of bacterial infection in mammals including humans.

The composition may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl phydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

The compound of formula (I) may be the sole therapeutic agent in the compositions of the invention or a combination with other antibacterials. If the other antibacterial is a β-lactam then a β-lactamase inhibitor may also be employed.

Compounds of formula (I) are active against a wide range of organisms including both Gram-negative and Gram-positive organisms.

The compounds of this invention may also be used in the manufacture of medicaments useful in treating bacterial infections in humans or other mammals.

All publications, including but not limited to patents and patent applications, cited in this specification are herein incorporated by reference (whether specifically stated to be so or not) as if each individual publication were specifically and individually indicated to be incorporated by reference herein as though fully set forth.

The following examples illustrate the preparation of certain compounds of formula (I) and the activity of certain compounds of formula (I) against various bacterial organisms. Although specific examples are described in the schemes, one of skill in the art appreciates that the methods are more generally applicable.

One of skill in the art readily appreciates that although the following schemes describe specific examples, they maybe more generally applied to produce additional embodiments of this invention. Furthermore, the examples set forth below are illustrative of the present invention and are not intended to limit, in any way, the scope of the present invention.

The compounds of the present invention were prepared by the methods illustrated in Schemes I, II, III and IV.

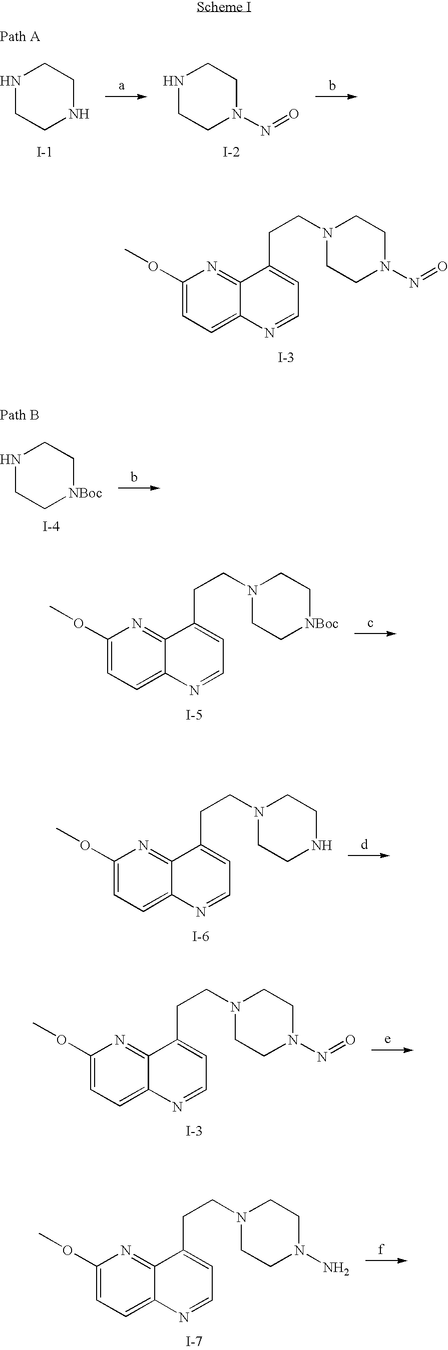

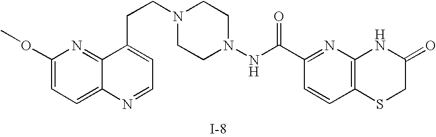

Reagents and conditions: (a) NH₂OH.HCl, potassium nitrosodisulfonate, 2% Na₂CO₃-pyr. (b) 8-ethenyl-2-(methyloxy)-1,5-naphthyridine, DMF, 90° C. (c) 4M HCl in dioxane, MeOH, 25° C. (d) Diazald® (N-Methyl-N-nitroso-p-toluenesulfonamide, Aldrich Chemical), DCM, reflux (e) lithium aluminum hydride (1M solution in tetrahydrofu ran), THF, 0-25° C. (f) N-(3-dimethylamino)propyl-N′-ethyl-carbodiimide, 1-hydroxybenzotriazole, 3-oxo-3,4-dihydro-2H-pyrido[3,2-b][1 ,4]thiazine-6-carboxylic acid, DCM-DMF, 25° C.

Nitrosamine (I-3) was prepared using two independent methods. In path A, commercial piperazine (I-1) was oxidized in a one-pot procedure (Vazques Tato, M. P.; Castedo, L.; Riguera, R. Chem. Lett. 1985, 623.) to the nitrosamine (I-2). The nitrosamine then underwent Michael addition into the vinyl substrate providing the adduct (I-3). The reaction proceeds most readily under high solvent concentration using protic or aprotic solvents, either EtOH or DMF. In path B, which was applicable to a wider variety of substrates, Boc-piperazine (I-4) underwent Michael addition as described above. The Boc group was removed under standard conditions, such as those described in standard references, such as Kocienski or Greene, cited previously herein. The free amine (I-6) was oxidized using excess Diazald® in either DCM or DCE at reflux providing the nitrosamine (I-3). Additionally, alternative nitrosating reagents could be employed (i.e. isoamyl nitrite). The nitrosamine (I-3) was subsequently reduced using a solution of lithium aluminum hydride in THF. The resulting hydrazine derivative (I-7) was unstable and therefore was used directly without further purification. The final hydrazide linkage was formed using standard coupling reagents.

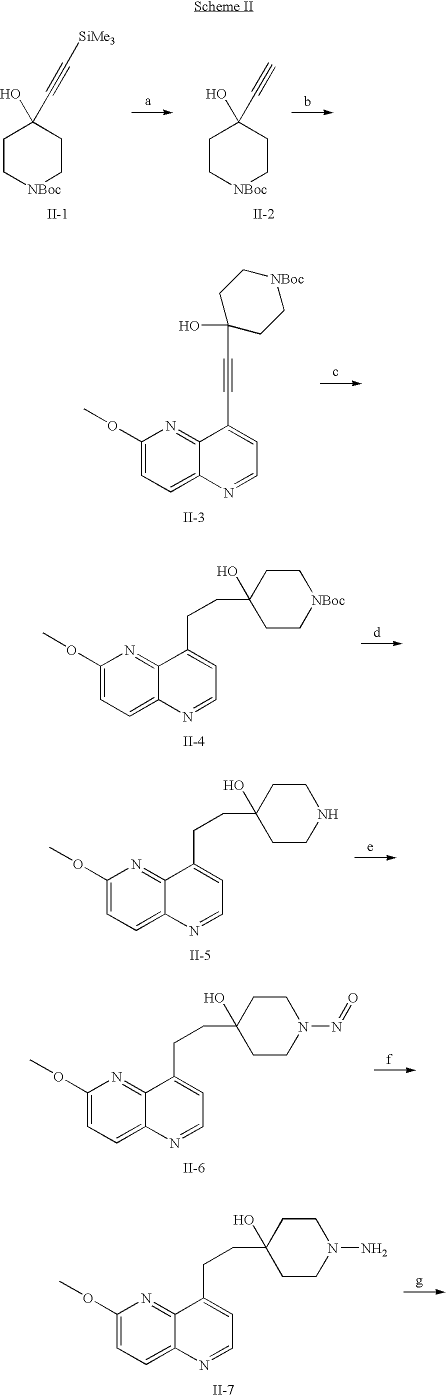

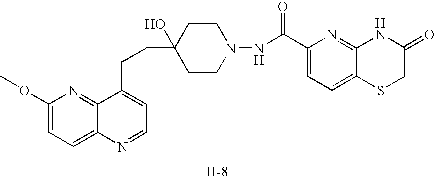

Reagents and conditions: (a) TBAF, THF, 0-25° C. (b) 6-(methyloxy)-1,5-naphthyridin-4-yl 4-methylbenzenesulfonate, CuI, DMF-Et₃N, Pd(PPh₃)₂Cl₂, microwave 120° C., 20 min (c) 10% Pd—C, EtOH, 50 psi of H₂ (d) 4M HCl in dioxane, MeOH, 25° C. (e) NH₂OH.HCl, potassium nitrosodisulfonate, 2% Na₂CO₃-pyr. (f) LAH, THF, 0-25° C. (g) N-(3-dimethylamino)propyl-N′-ethyl-carbodiimide, 1-hydroxybenzotriazole, 3-oxo-3,4-dihydro-2H-pyrido[3,2-b][1,4]thiazine-6-carboxylic acid, DCM-DMF, 25° C.

The piperidine (II-1) (Prepared according to van Niel, M. B.; et al. J. Med. Chem. 1999, 42, 12, 2087) was treated with an appropriate fluoride source removing the silyl protecting group and affording the free alkyne (II-2). Subsequent coupling of the alkyne using the standard Sonagashira coupling protocol (Sonogashira, K.; Tohda, Y.; Hagihara, N. Tetrahedron Lett. 1975, 4467), under microwave conditions, provided the alkyne (II-3). Hydrogenation with Pd—C provided the saturated substrate (II-4). The Boc group was removed under standard conditions, such as those described in standard references, such as Kocienski or Greene, cited previously herein. The resulting piperidine (II-5) was directly oxidized to the nitrosamine (II-6) using the oxidation methods described in Scheme 1. Reduction and acid coupling , conditions identical to that described in Scheme 1, provided the hydrazide (II-8).

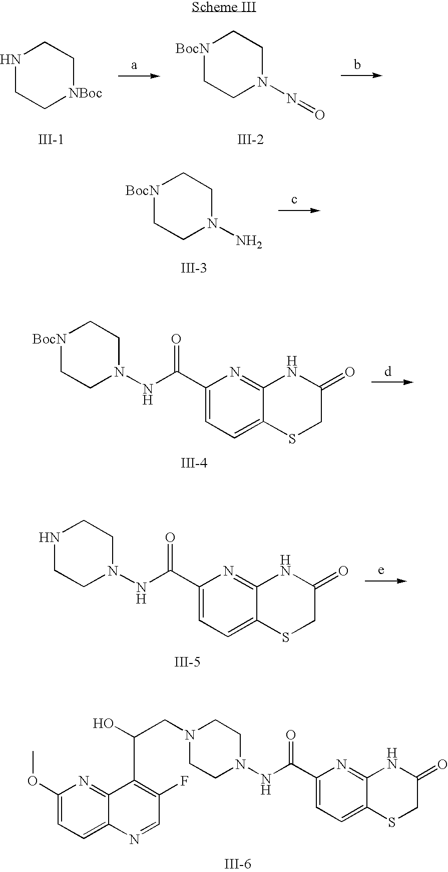

Reagents and conditions: (a) Diazald®, DCM, reflux (b) LAH, THF, 0-25° C. (c) N-(3-dimethylamino)propyl-N′-ethyl-carbodiimide, 1-hydroxybenzotriazole, 3-oxo-3,4-dihydro-2H-pyrido[3,2-b][1,4]thiazine-6-carboxylic acid, DCM-DMF, 25° C. (d) 4M HCl in dioxane, MeOH, 25° C. (e) 7-fluoro-2-(methyloxy)-8-(2-oxiranyl)-1,5-naphthyridine, DMF, 90° C.

Boc-piperazine (III-1) was oxidized to the nitrosamine (III-2) using Diazald® as described in Scheme 1. Reduction and acid coupling, conditions like that described in Scheme 1, provided the hydrazide (III-4). The Boc group was removed under standard conditions, such as those described in standard references, such as Kocienski or Greene, cited previously herein. Subsequent epoxide opening using free amine (III-5) yielded the final compound (III-6).

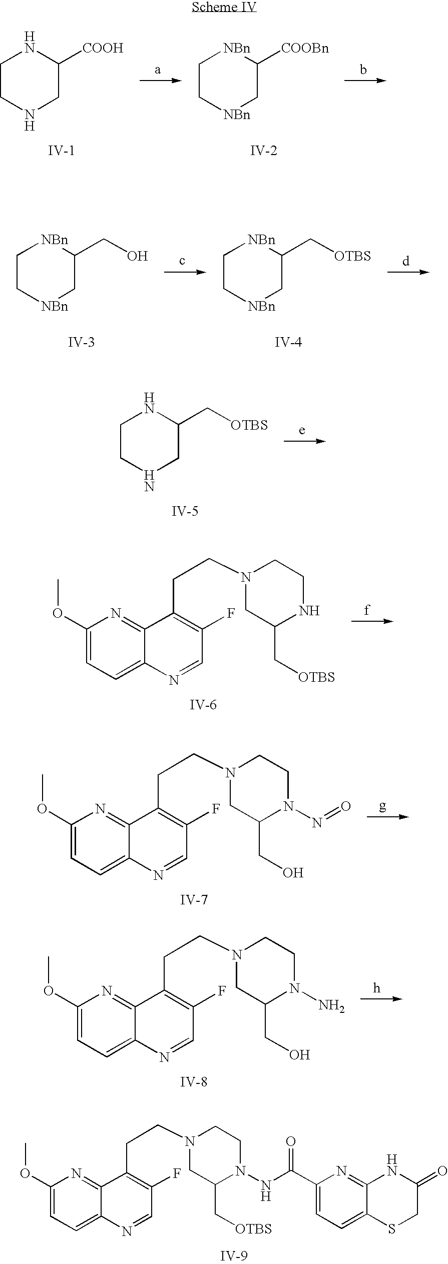

Reagents and conditions: (a) benzyl chloride, triethylamine, dioxane-DMF, 100° C. (b) LAH, THF, 0° C. (c) triethylamine, tert-butyidimethylchlorosilane, DMAP, DCM, 0° C. (d) 20% Pd(OH)₂, EtOH, 50 psi of H₂ (e) 8-ethenyl-7-fluoro-2-(methyloxy)-1,5-naphthyridine, EtOH, 90° C. (f) Diazald®, DCE, reflux (g) LAH, THF, 0° C. (h) N-(3-dimethylamino)propyl-N′-ethyl-carbodiimide, 1-hydroxybenzotriazole, 3-oxo-3,4-dihydro-2H-pyrido[3,2-b][1,4]thiazine-6-carboxylic acid, DCM-DMF, 25° C.

2-piperazinecarboxylic acid treated with excess benzyl chloride. The resulting benzyl ester was reduced to alcohol (IV-3) which was subsequently protected as the silyl ether. The remaining benzyl groups were removed through providing the free amine (IV-5), which then underwent Michael addition into the vinyl substrate providing the adduct (IV-6) as described in Scheme 1. Piperazine (IV-6) was oxidized using excess Diazald® providing the nitrosamine (IV-7). Treatment with LAH reduced the nitrosamine and removed the TBS protecting group. The free amine (IV-8) was then coupled with the acid under conditions described in Scheme 1 to afford the final compound (IV-9).

### General

Proton nuclear magnetic resonance (¹H NMR) spectra were recorded at 400 MHz, and chemical shifts are reported in parts per million (δ) downfield from the internal solvent standard CHCl₃ or MeOH. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. CDCl₃ is deuteriochloroform and CD₃OD is tetradeuteriomethanol. Mass spectra were obtained using electrospray (ES) ionization techniques. All temperatures are reported in degrees Celsius. E. Merck Silica Gel 60 F-254 thin layer plates were used for thin layer chromatography. Flash chromatography was carried out on E. Merck Kieselgel 60 (230-400 mesh) silica gel. Analytical HPLC was performed on Beckman chromatography systems. Preparative HPLC was performed using Gilson chromatography systems. ODS refers to an octadecylsilyl derivatized silica gel chromatographic support. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev. Celite® is a filter aid composed of acid-washed diatomaceous silica, and is a registered trademark of Manville Corp., Denver, Colo.

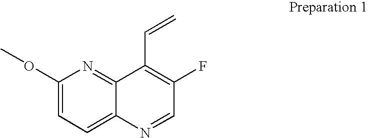

### Preparation of 8-ethenyl-7-fluoro-2-(methyloxy)-1,5-naphthyridine

### a) (2-[(6-Methoxypyridin-3-ylamino)methylene]malonic acid diethyl ester

A solution of 5-amino-2-methoxypyridine (Aldrich, 100 g, 0.806 mole) and diethyl ethoxymethylenemalonate (Aldrich, 163 mL, 0.806 mole) in EtOH (1 L) was heated at reflux for 4 hours, then was cooled to RT. Concentration to dryness gave the title compound (238 g, quantitative).

### b) 6-Methoxy-4-oxo-1,4-dihydro-[1,5]naphthyridine-3-carboxylic acid ethyl ester

Dowtherm A (Fluka, 500 mL) was brought to boiling (250° C. in a 2 L 3-neck flask fitted with a still-head and a reflux condenser. 2-[(6-Methoxypyridin-3-ylamino)methylene]malonic acid diethyl ester (100 g, 0.34 mole) was added portion-wise over 5 min. The solution was heated at reflux for an additional 15 min, allowing some solvent to distil over. The resulting solution was cooled to RT and diluted with hexanes (750 mL). The mixture was cooled in ice for 1 hr, then the brown solid was filtered off, washed with hexanes, and dried under vacuum to afford the title compound (61.72 g, 73%).

### c) 4-Bromo-6-methoxy-[1,5]naphthyridine-3-carboxylic acid ethyl ester

A suspension of 6-methoxy-4-oxo-1,4-dihydro-[1,5]naphthyridine-3-carboxylic acid ethyl ester (74.57 g, 300 mmole) in dry DMF (260 mL) under argon was stirred efficiently* in a water bath (to maintain approximately RT—may need slight ice-cooling on a large scale). Phosphorus tribromide (30.0 mL, 316 mmole) was added dropwise over 15 min and stirring was continued for an additional 30 min. Water (1 L) was added, followed by saturated sodium carbonate solution to pH 7. The solid was collected by suction filtration, washed with water and dried under vacuum over phosphorus pentoxide to give the title compound (83.56 g, 90%).

### d) 4-Bromo-6-methoxy-[1,5]naphthyridine-3-carboxylic acid

2 N NaOH (300 mL, 600 mmole) was added dropwise over 30 min to a stirred solution of 4-bromo-6-methoxy-[1,5]naphthyridine-3-carboxylic acid ethyl ester (83.56 g, 268 mmole) in THF (835 mL). Stirring was continued overnight, at which time LC/MS showed that the saponification was complete. 2 N HCl was added to pH 6 and the THF was removed in vacuo. 2 N HCl was added to pH 2, then water (250 mL) was added, and the mixture was cooled thoroughly in ice. The solid was collected by suction filtration, washed with water and dried (first using a rotary evaporator at 50° C. and then under high vacuum at 50° C. overnight) to give the title compound (76.7 g, slightly over quantitative). This material was used without further purification.

### e) 4-Bromo-6-methoxy-[1,5]naphthyridin-3-ylamine

A suspension of 4-bromo-6-methoxy-[1,5]naphthyridine-3-carboxylic acid (50 g, 177 mmole) in dry DMF (600 mL) was treated with triethylamine (222.5 mL, 1.60 mole), tert-butanol (265 mL, 2.77 mole), and diphenylphosphoryl azide (41.75 mL, 194 mmole). The reaction was stirred under argon at 100° C. for 1 hr, then was cooled to RT and concentrated to low volume. Ethyl acetate and excess aqueous sodium bicarbonate solution were added, the mixture was shaken, and some insoluble solid was filtered off. The layers were separated and the organic phase was washed with water (2×) and dried (MgSO₄). Concentration to dryness gave a crude mixture of 4-bromo-6-methoxy-[1,5]naphthyridin-3-ylamine (minor product) and (4-bromo-6-methoxy-[1,5]naphthyridin-3-ylamine)carbamic acid tert-butyl ester (major product) along with impurities.

Without further purification, this mixture was dissolved in CH₂Cl₂ (150 mL) and treated with trifluoroacetic acid (100 mL). The reaction was stirred for 3 hr then was concentrated to dryness. The residue was partitioned between CHCl₃ and saturated sodium bicarbonate solution and the layers were separated. The aqueous phase was extracted with CHCl₃, and the combined organics were dried (MgSO₄) and concentrated to low volume. The solid was collected by suction filtration, washed with a small volume of CHCl₃ and dried under vacuum to afford a first crop of the title compound (31.14 g). The filtrate was purified by flash chromatography on silica gel (30% EtOAc/CHCl₃) to afford further material (2.93 g, total=34.07 g, 76%). Alternatively, the filtrate was left at RT overnight and then filtered to give a second crop of the title compound (2.5 g).

### f) 4-Bromo-6-methoxy-[1,5]naphthyridine-3-diazonium tetrafluoroborate

A solution of 4-bromo-6-methoxy-[1,5]naphthyridin-3-ylamine (25.2 g, 99.2 mmole) in dry THF (400 mL) was maintained at −5° C. while nitrosonium tetrafluoroborate (12.9 g, 110 mmole) was added portion-wise over 30 min (approximately 2 g portions). The reaction was continued for an additional 1 hr at -5° C., at which time TLC* and LC/MS indicated that the reaction was complete. The orange solid was collected by suction filtration, washed with ice-cold THF and dried under vacuum to provide the title compound (31.42 g, 90%).

### g) 4-Bromo-3-fluoro-6-methoxy-[1,5]naphthyridine

A suspension of 4-bromo-6-methoxy-[1,5]naphthyridine-3-diazonium tetrafluoroborate (31.42 g, 89.0 mmole) in decalin (mixed isomers, 500 mL) in a 2 L flask* was heated to 180° C. and held at this temperature for 5 min. The mixture was cooled and diluted with CHCl₃ (500 mL, to keep the product in solution), and the resulting mixture was stirred vigorously for 30 min to break up a black solid by product. The mixture was then poured onto a column of silica gel and the column was eluted with CHCl₃ to remove decalin and then with 3% EtOAc/CHCl₃ to afford the title compound (9.16 g, 40%).

### h) 8-ethenyl-7-fluoro-2-(methyloxy)-1,5-naphthyridine

To a solution of 8-bromo-7-fluoro-2-(methyloxy)-1,5-naphthyridine (2.0 g, 7.81 mmol), potassium carbonate (1.08 g, 7.81 mmole), tetrakis-triphenylphosphine (90 mg, 0.08 mmole) in DME (60 mL) and H₂O (20 mL) was added 2,4,6-trivinylcycloborane-pyridine complex (0.94 g, 3.91 mmole). After stirring for 10 hours at 85° C. the reaction contents were concentrated and the product purified by chromatography on silica gel (hexanes/EtOAc, 4:1) to give a low melting solid (1.43 g, 90%).

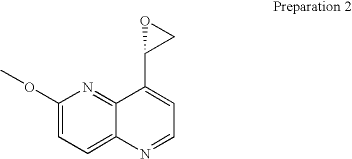

### Preparation of (S)-2-(6-Methoxy-[1,5]-naphthyridin-4-yl)oxirane

### (a) 4-Hydroxy-6-methoxy-[1,5]-naphthyridine

5-Amino-2-methoxypyridine (55 g, 0.44 mol) in methanol (1000 ml) with methyl propiolate (40 ml, 0.44 mol) was stirred for 48 h, then evaporated and the product purified by chromatography on silica gel (DCM) followed by recrystallisation from DCM-hexane (44.6 g, 48%).

The unsaturated ester (10.5 g, 0.05 mol) in warm Dowtherm A (50 ml) was added over 3 minutes to refluxing Dowtherm A, and after a further 20 minutes at reflux the mixture was cooled and poured into ether. The precipitate was filtered to give the title compound (6.26 g, 70%)

### (b) Bromomethyl-(6-methoxy-[1,5]-naphthyridin-4-yl)-ketone

4-Hydroxy-6-methoxy-[1,5]-naphthyridine (10 g, 0.057 mol) in DCM (200 ml) containing 2,6-lutidine (9.94 ml, 0.086 mol) and 4-dimethylaminopyridine (0.07 g, 0.0057 mol) was cooled in ice and treated with trifluoromethanesulfonic anhydride (10.5 ml, 0.063 mol). After stirring for 2.5 h the mixture was washed with saturated ammonium chloride solution, dried, evaporated and purified on silica (DCM). The triflate (13.2 g, 0.044 mol) in DMF (200 ml) with TEA (12 ml, 0.086 mol), butyl vinyl ether (22 ml, 0.17 mol), 1,3-bis(diphenylphosphino)propane (1.77 g, 0.0044 mol) and palladium (II) acetate (0.97 g, 0.0044 mol) was heated at 60° C. for 3 h then evaporated and chromatographed on silica gel (DCM) to give a yellow solid (10.7 g, 95%). This was dissolved in THF (250 ml), water (40 ml) and treated with N-bromosuccinimide (7.4 g. 0.042 mol) for 1 h, then evaporated and chromatographed on silica gel (DCM) to give the ketone (10.42 g, 98%).

### c) (R)-2-Bromo-1-(6-methoxy-[1,5]-naphthyridin-4-yl)ethanol

Bromomethyl-(6-methoxy-[1,5]-naphthyridin-4-yl)-ketone (6.6 g, 0.023 mol) in toluene was treated with (+)-B-chlorodiisopinocamphenylborane ((+)-DIP-chloride) (12 g, 0.037 mol) and stirred overnight, then diethanolamine (15 g, 0.14 mol) added and the mixture stirred for 3 hours, filtered and evaporated. Chromatography on silica gel (ethyl acetate-hexane )gave a white solid (4.73 g, 73%).

### d) (R)-2-(6-Methoxy-[1,5]-naphthyridin-4-yl)oxirane

(R)-2-Bromo-1-(6-methoxy-[1,5]-naphthyridin-4-yl)ethanol (4.8 g, 0.017 mol) in methanol (20 ml) was stirred with potassium carbonate (2.6 g, 0.019 mol) for 1 hour, then evaporated and chromatographed on silica gel (ethyl acetate-hexane-dichloromethane) to give a solid (3.14 g, 92%), (91% ee by chiral HPLC). MS (+ve ion electrospray) m/z 203 (M+H+).

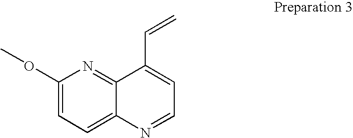

### Preparation of 8-ethenyl-2-(methyloxy)-1,5-naphthyridine

To a solution of 6-(methyloxy)-1,5-naphthyridin-4-yl trifluoromethanesulfonate (from Prep. 2b) (5.0 g, 16.23 mmole) in DME (80 mL) and H₂O (40 mL) was added trivinyl boronate (1.96 g, 8.1 mmole), K₂CO₃ (2.23 g, 16.23 mmole) and Pd(PPh₃)₄ (0.19 g, 0.16 mmole). After 3 h at 90° C. under N₂, the reaction solution was concentrated under vacuum and purified on silica (hexanes, EtOAc, 4:1) to give a yellow oil (2.44 g, 81%): LC-MS (m/z) (ES) 187 (M+H)⁺.

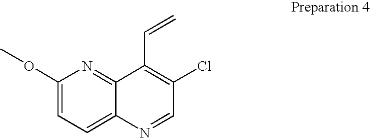

### Preparation of 7-chloro-8-ethenyl-2-(methyloxy)-1,5-naphthyridine

### a) 3-Chloro-6-methoxy-[1,5]naphthyridin-4-ol

6-Methoxy-[1,5]naphthyridin-4-ol (12 g) in acetic acid (200 mL) was sonicated and warmed until all had dissolved, and then it was treated with N-chlorosuccinimide (10.01 g) and the mixture was heated at 35° C. for 18 hr, cooled, and the solid collected and washed with acetic acid and dried in vacuo at 40° C. overnight, to give a white solid (9.5 g); MS (ES) m/z 211/213 (M+H)⁺.

### b) 1,1,1-Trifluoro-methanesulfonic acid 3-chloro-6-methoxy-[1,5]naphthyridin-4-yl ester

A suspension of 60% sodium hydride in oil (3.08 g) was washed with hexane, the hexane solution decanted, and dry DMF (200 mL) added followed by the phenol (1a) (11.62 g). The mixture was stirred at room temperature for 1 hr, cooled in ice, N-phenyltrifluoromethanesulphonimide (21.62 g) added and the mixture was allowed to stir at room temperature overnight. It was evaporated, azeotroped with toluene, taken up in ether-DCM and washed with sodium carbonate solution, dried (sodium sulfate) and evaporated to give a solid (15 g); MS (+ve ion electrospray) m/z 343/345 (MH+).

### c) 7-Chloro-2-methoxy-8-vinyl-[1,5]naphthyridine

1,1,1-Trifluoro-methanesulfonic acid 3-chloro-6-methoxy-[1,5]naphthyridin-4-yl ester (1 g) in DME (20 mL) under argon, was treated with tetrakis(triphenylphosphine)palladium(0) (0.21 g) and the mixture stirred at room temperature for 20 minutes. Anhydrous potassium carbonate (0.403 g), water (6 mL), and vinylborane:pyridine complex (see F. Kerins and D O'Shea J. Org. Chem. 2002, 67, 4968-4971) (1.056 g) were added and the mixture was heated at 100° C. for 1.5 hr. It was cooled, diluted with water and extracted with ether, dried (sodium sulfate), evaporated and chromatographed on silica gel, eluting with DCM then chloroform to afford a white solid (0.53 g); MS (ES) m/z 221/223 (M+H)⁺.

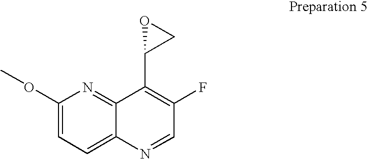

### Preparation of 7-fluoro-2-(methyloxy)-8-[(2S)-2-oxiranyl]-1,5-naphthyridine

### a) 1-[3-fluoro-6-(methoxy)-1,5-naphthyridin-4-yl]-1,2-ethanediol

To a solution of AD-mix-β(50 g) in tert-butanol/water (200 ml/200 mL), cooled in an ice-bath for 30 minutes, 8-ethenyl-7-fluoro-2-(methyloxy)-1,5-naphthyridine (prepared as Preparation 1) (8 g, 39.2 mmol) was added and the reaction mixture was stirred at room temperature for 48 hours. Sodium sulfite (75 g) was added and the mixture was stirred for a further 30 minutes. It was extracted with diethyl ether then several times with 10% methanol in chloroform. The organic extract was evaporated under vacuum to afford the desired product as an oil (8.93 g, 96%). MS (+ve ion electrospray) m/z 239 (MH+). enantiomeric excess=44%, as determined by chiral analytical HPLC

### b) 2-[3-fluoro-6-(methoxy)-1,5-naphthyridin-4-yl]-2-hydroxyethyl 4-methylbenzenesulfonate

To a solution of diol (a) (16.5 g, 6.93 mmol) in DCM (200 mL), triethylamine (10 mL) and dibutyltin oxide (350 mg) was added tosyl chloride (13.2 g, 6.94 mmol). After 3 hours, the mixture was diluted with water/sodium bicarbonate and extracted several times with chloroform. The combined organic extracts were dried over magnesium sulfate and evaporated under vacuum. The residue was chromatographed on silica gel eluting with 20-30% ethyl acetate in chloroform to afford the desired product (20.3 g, 75%). MS (+ve ion electrospray) m/z 393 (MH+).

### c) 7-fluoro-2-(methoxy)-8-(2-oxiranyl)-1,5-naphthyridine

To a suspension of tosylate (b) (10.5 g, 26.7 mmol) in anhydrous methanol (160 mL), cooled in an ice-bath, potassium carbonate (7.03 g, 50.9 mmol) was added. After 15 minutes with cooling, the mixture was stirred at room temperature for a further 1.75 hours. It was then diluted with water, extracted several times with dichloromethane, dried over magnesium sulfate and evaporated under vacuum. The residue was chromatographed on silica gel eluting with dichloromethane, chloroform then 20% ethyl acetate in chloroform to afford the title product as an oil (5.55 g, 94%). MS (+ve ion electrospray) m/z 221 (MH+).

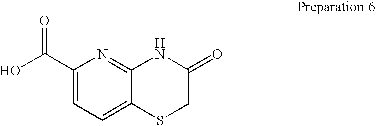

### Preparation of 3-oxo-3,4-dihydro-2H-pyrido[3,2-b][1,4]thiazine-6-carboxylic acid

### a) Methyl 3-oxo-3,4-dihydro-2H-pyrido[3,2-b][1,4]thiazine-6-carboxylate

A solution of ethyl 2-mercaptoacetate (1.473 mL) in DMF (48 mL) was ice-cooled and treated with sodium hydride (540 mg of a 60% dispersion in oil). After 1 hour methyl 6-amino-5-bromopyridine-2-carboxylate (3 g) (T. R. Kelly and F. Lang, J. Org. Chem. 61, 1996, 4623-4633) was added and the mixture stirred for 16 hours at room temperature. The solution was diluted with EtOAc (1 litre), washed with water (3×300 mL), dried and evaporated to about 10 mL. The white solid was filtered off and washed with a little EtOAc to give the ester (0.95 g); MS (APCI⁻) m/z 223 ([M−H]⁻, 100%).

### b) 3-Oxo-3,4-dihydro-2H-pyrido[3,2-b][1,4]thiazine-6-carboxylic acid

A solution of Methyl 3-oxo-3,4-dihydro-2H-pyrido[3,2-b][1,4]thiazine-6-carboxylate (788 mg) in dioxan (120 ml)/water (30 mL) was treated dropwise over 2 hours with 0.5M NaOH solution (8 mL) and stirred overnight. After evaporation to approx. 3 ml, water (5 mL) was added and 2M HCl to pH4. The precipitated solid was filtered off, washed with a small volume of water and dried under vacuum to give a solid (636 mg); MS (APCI⁻) m/z 209 ([M−H]⁻, 5%), 165([M−COOH]⁻, 100%).

### c) 6-Hydroxymethyl-3-oxo-3,4-dihydro-2H-pyrido[3,2-b][1,4]thiazine

A solution of 3-Oxo-3,4-dihydro-2H-pyrido[3,2-b][1,4]thiazine-6-carboxylic acid (500 mg) in THF (24 mL) with triethylamine (0.396 mL) was cooled to −10° C. and isobutyl chloroformate (0.339 ml) added. After 20 minutes the suspension was filtered through kieselguhr into an ice-cooled solution of sodium borohydride (272 mg) in water (8 mL), the mixture stirred 30 minutes and the pH reduced to 7 with dilute HCl. The solvent was evaporated and the residue triturated under water. The product was filtered and dried under vacuum to give a white solid (346 mg); MS (APCI⁻) m/z 195 ([M−H]⁻, 50%), 165(100%).

### d) 3-Oxo-3,4-dihydro-2H-pyrido[3,2-b][1,4]thiazine-6-carboxaldehyde

A solution of 6-Hydroxymethyl-3-oxo-3,4-dihydro-2H-pyrido[3,2-b][1,4]thiazine (330 mg) in dichloromethane (30 mL)/THF (30 mL) was treated with manganese dioxide (730 mg) and stirred at room temperature. Further manganese dioxide was added after 1 hour (730 mg) and 16 hours (300 mg). After a total of 20 hours the mixture was filtered through kieselguhr and the filtrate evaporated. The product was triturated with EtOAc/hexane (1:1) and collected to give a solid (180 mg); MS (APCI⁻) m/z 195 ([M−H]⁻, 95%), 165 (100%).

### e) 3-oxo-3,4-dihydro-2H-pyrido[3,2-b][1,4]thiazine-6-carboxylic acid

This acid was prepared from 3-Oxo-3,4-dihydro-2H-pyrido[3,2-b][1,4]thiazine-6-carboxaldehyde (890 mg) by oxidation with Oxone (potassium peroxymonosulphate) (3.1 g) in a DMF solution (50 mL). After 1.5 hours at room temperature, dilution with water (50 mL) filtration and drying in vacuo afforded the acid as a white solid (750 mg, 77%).

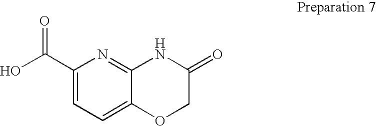

### Preparation of 3-oxo-3,4-dihydro-2H-pyrido[3,2-b][1,4]oxazine-6-carboxylic acid

### a) 2-Bromo-5-hydroxy-6-nitropyridine

3-Hydroxy-2-nitropyridine (20 g, 0.143 mole) was dissolved in methanol (400 mL) and a solution of 25% sodium methoxide in methanol (33 mL, 0.13 mole) was added at room temperature. The mixture was stirred for 30 min, then was cooled to 0° C., and bromine (7.2 mL, 0.14 mole) was added slowly. The reaction was stirred at 0° C. for 30 min, then was quenched with glacial AcOH (2.5 mL). The solvent was removed in vacuo to afford material (30 g, 96%), which was used without further purification. MS (ES) m/z 219.0 (M+H)⁺.

### b) Ethyl (6-bromo-2-nitro-pyridin-3-yloxy)acetate

2-Bromo-5-hydroxy-6-nitropyridine (30 g, 0.14 mole) was suspended in acetone (200 ml), and potassium carbonate (39 g, 0.28 mole) was added, followed by ethyl bromoacetate (15.7 ml, 0.14 mmole). The reaction was heated at reflux for 10 hr, then was cooled to room temperature and diluted with Et₂O. The precipitate was removed by suction filtration, and the filtrate was concentrated in vacuo to afford material (38 g, 89%), which was used without further purification; MS (ES) m/z 305.0 (M+H)⁺.

### c) 6-Bromo-4H-pyrido[3,2-b][1,4]oxazin-3-one

Ethyl (6-bromo-2-nitro-pyridin-3-yloxy)acetate (38 g, 0.125 mole) was dissolved in glacial AcOH (150 mL), and iron powder (20 g, 0.36 mole) was added. The mixture was mechanically stirred and heated at 90° C. for 5 hr, then was cooled to room temperature and diluted with EtOAc (300 mL). The mixture was filtered through a pad of silica gel and the filtrate was concentrated in vacuo and the residue recrystallized from MeOH (15 g, 52%); MS (ES) m/z 229.0 (M+H)⁺.

### d) 6-((E)-Styryl)-4H-pyrido[3,2-b][1,4]oxazin-3-one

6-Bromo-4H-pyrido[3,2-b][1,4]oxazin-3-one (6.0 g, 26.3 mmole) and trans-2-phenylvinylboronic acid (3.9 g, 26.3 mmole) were dissolved in 1,4-dioxane (150 mL) and the solution was degassed with argon. (Ph₃P)₄Pd (230 mg, 0.2 mmole) was added, followed by a solution of potassium carbonate (6.9 g, 50 mmole) in H₂O (20 mL). The reaction was heated at reflux under argon overnight, then was cooled to room temperature and diluted with EtOAc (200 mL). The solution was washed sequentially with H₂O and brine, dried (Na₂SO₄), and concentrated in vacuo. The solid residue was purified by flash chromatography on silica gel (5-10% EtOAc/CHCl₃) to afford a solid (2.5 g, 38%): LCMS (ES) m/z 253.0 (M+H)⁺.

### e) 3-Oxo-3,4-dihydro-2H-pyrido[3,2-b][1,4]oxazine-6-carboxaldehyde

6-((E)-Styryl)-4H-pyrido[3,2-b][1,4]oxazin-3-one (1.2 g, 4.8 mmole) was dissolved in CH₂Cl₂ (200 mL) and the solution was cooled to −78° C. Ozone was bubbled through the solution with stirring until a pale blue color appeared, then the excess ozone was removed by bubbling oxygen through the solution for 15 min. Dimethylsulfide (1.76 mL, 24 mmole) was added to the solution, and the reaction was stirred at −78° C. for 3 hr, then at room temperature overnight. The solvent was removed in vacuo, and the residue was triturated with Et₂O (50 mL). The collected solid was washed with additional Et₂O and dried to afford a solid (700 mg, 82%): MS (ES) m/z 179.0 (M+H)⁺.

### f) 3-oxo-3,4-dihydro-2H-pyrido[3,2-b][1,4]oxazine-6-carboxylic acid

This acid was prepared from 3-Oxo-3,4-dihydro-2H-pyrido[3,2-b][1,4]oxazine-6-carboxaldehyde (900 mg) by oxidation with Oxone (potassium peroxymonosulphate) (3.7 g) in a DMF solution (50 mL). After 1.5 hours at room temperature, dilution with water (50 mL) filtration and drying in vacuo afforded the acid as an off-white solid (687 mg, 70%): LCMS (ES) m/e 195 (M+H)⁺; ¹H NMR δ 7.81 (d, J=8.1 Hz, 1H), 7.41 (d, J=8.1 Hz, 1H), 4.77 (s, 2H).

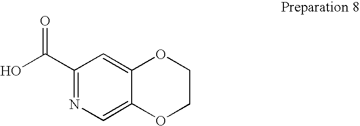

### Preparation of 2,3-dihydro[1,4]dioxino[2,3-c]pyridine-7-carboxylic acid

### a) 5-Benzyloxy-2-hydroxymethyl-1H-pyridin-4-one

A mixture of 5-benzyloxy-2-hydroxymethyl-4-pyrone (prepared from Kojic acid by the method of D. Erol, J. Med. Chem., 1994, 29, 893) (9.7 g, 40 mmol), concentrated aqueous (880) ammonia (100 mL), and ethanol (20 mL) was heated to reflux overnight. The mixture was allowed to cool to room temperature then filtered. The resultant solid was washed with ether and dried in vacuo (5.9 g); MS (APCl+) m/z 232 (MH+).

### b) (2,3-Dihydro-[1,4]dioxino[2,3-c]pyridin-7-yl)-methanol

A solution of 5-Benzyloxy-2-hydroxymethyl-1H-pyridin-4-one (2 g, 8.7 mmol) in water (220 mL) containing sodium hydroxide (17 mmol) was hydrogenated over 10% palladium on charcoal (1 g) for 4 hours. The mixture was filtered and evaporated to give a white solid. This solid was dissolved in N,N-dimethylformamide (8 mL) then treated with potassium carbonate (2.9 g) and 1,2-dibromoethane (0.6 mL, 7 mmol). The mixture was heated at 85° C. overnight. The cooled mixture was evaporated onto silica and chromatographed eluting with 10-30% methanol in ethyl acetate affording a white solid (250 mg, 21%); MS (APCl+) m/z 168 (MH+).

### c) 2,3-Dihydro-[1,4]dioxino[2,3-c]pyridine-7-carboxaldehyde

A solution of (2,3-Dihydro-[1,4]dioxino[2,3-c]pyridin-7-yl)-methanol (250 mg, 1.5 mmol) in dichloromethane (5 mL) was treated with manganese dioxide (650 mg, 7.5 mmol). After 3 days the mixture was filtered and evaporated affording a white solid (150 mg, 61%); MS (APCl+) m/z 166 (MH+).

### d) 2,3-dihydro[1,4]dioxino[2,3-c]pyridine-7-carboxylic acid

To a solution of 2,3-Dihydro-[1,4]dioxino[2,3-c]pyridine-7-carboxaldehyde (780 mg, 4.74 mmol) in acetone-H₂O (1:4, 47 mL) at 25° C. were added NaClO₂ (575 mg, 6.36 mmol) followed by H₂NSO₃H (599 mg, 6.17 mmol). After 2 h, the solution was concentrated and the residue purified through a plug of silica (10% MeOH in DCM (1% NH₄OH)) affording the title compound as an off-white solid (600 mg, 70%): LCMS (ES) m/e 182 (M+H)⁺; ¹H NMR (CD₃OD, 400 MHz) δ 8.11 (s, 1H), 7.57 (s, 1H), 4.37-4.92 (m, 4H).

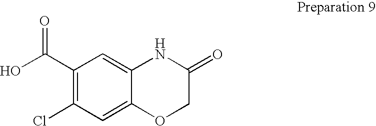

### Preparation of 7-chloro-3-oxo-3,4-dihydro-2H-1,4-benzoxazine-6-carboxylic acid

### a) 2-Bromo-5-hydroxy-6-nitropyridine

3-Hydroxy-2-nitropyridine (20 g, 0.143 mole) was dissolved in methanol (400 mL) and a solution of 25% sodium methoxide in methanol (33 mL, 0.13 mole) was added at room temperature. The mixture was stirred for 30 min, then was cooled to 0° C., and bromine (7.2 mL, 0.14 mole) was added slowly. The reaction was stirred at 0° C. for 30 min, then was quenched with glacial AcOH (2.5 mL). The solvent was removed in vacuo to afford material (30 g, 96%), which was used without further purification. MS (ES) m/z 219.0 (M+H)⁺.

### b) Ethyl (6-bromo-2-nitro-pyridin-3-yloxy)acetate

The hydroxypyridine (30 g, 0.14 mole) was suspended in acetone (200 ml), and potassium carbonate (39 g, 0.28 mole) was added, followed by ethyl bromoacetate (15.7 ml, 0.14 mmole). The reaction was heated at reflux for 10 hr, then was cooled to room temperature and diluted with Et₂O. The precipitate was removed by suction filtration, and the filtrate was concentrated in vacuo to afford material (38 g, 89%), which was used without further purification: LCMS (ES) m/z 305.0 (M+H)⁺.

### c) 6-Bromo-4H-pyrido[3,2-b][1,4]oxazin-3-one

The nitropyridine (38 g, 0.125 mole) was dissolved in glacial AcOH (150 mL), and iron powder (20 g, 0.36 mole) was added. The mixture was mechanically stirred and heated at 90° C. for 5 hr, then was cooled to room temperature and diluted with EtOAc (300 mL). The mixture was filtered through a pad of silica gel and the filtrate was concentrated in vacuo and the residue recrystallized from MeOH (15 g, 52%): LCMS (ES) m/z 229.0 (M+H)⁺.

### d) 6-((E)-Styryl)-4H-pyrido[3,2-b][1,4]oxazin-3-one

The bromopyridine (10c) (6.0 g, 26.3 mmole) and trans-2-phenylvinylboronic acid (3.9 g, 26.3 mmole) were dissolved in 1,4-dioxane (150 mL) and the solution was degassed with argon. (Ph₃P)₄Pd (230 mg, 0.2 mmole) was added, followed by a solution of potassium carbonate (6.9 g, 50 mmole) in H₂O (20 mL). The reaction was heated at reflux under argon overnight, then was cooled to room temperature and diluted with EtOAc (200 mL). The solution was washed sequentially with H₂O and brine, dried (Na₂SO₄), and concentrated in vacuo. The solid residue was purified by flash chromatography on silica gel (5-10% EtOAc/CHCl₃) to afford a solid (2.5 g, 38%): LCMS (ES) m/z 253.0 (M+H)⁺.

### e) 3-Oxo-3,4-dihydro-2H-pyrido[3,2-b][1,4]oxazine-6-carboxaldehyde

The pyridine (10d) (1.2 g, 4.8 mmole) was dissolved in CH₂Cl₂ (200 mL) and the solution was cooled to −78° C. Ozone was bubbled through the solution with stirring until a pale blue color appeared, then the excess ozone was removed by bubbling oxygen through the solution for 15 min. Dimethylsulfide (1.76 mL, 24 mmole) was added to the solution, and the reaction was stirred at −78° C. for 3 hr, then at room temperature overnight. The solvent was removed in vacuo, and the residue was triturated with Et₂O (50 mL). The collected solid was washed with additional Et₂O and dried to afford a solid (700 mg, 82%); MS (ES) m/z 179.0 (M+H)⁺.

### f) 6-Bromo-7-chloro-4H-pyrido[3,2-b][1,4]oxazin-3-one

6-Bromo-4H-pyrido[3,2-b][1,4]oxazin-3-one (20 g, 87.7 mmole) was dissolved in DMF (175 mL) and cooled in an ice bath. Chlorine gas was then slowly bubbled in for 45 minutes, and then the saturated solution was stirred in the ice bath for 2 hours. The mixture was purged with nitrogen and slowly added with stirring to 1 L of ice water which contained 100 g of Na₂SO₃, making sure to keep the temperature <15° C. After stirring 30 minutes the product was filtered, washed thoroughly with water and dried to afford (22.5 g, 98%) of a white solid. ¹H NMR (400 MHz, DMSO-d6) δ 4.76 (2H, s,), 7.78 (1H, s), 11.71 (1H, s).

### g) 7-Chloro-6-((E)-styryl)-4H-pyrido[3,2-b][1,4]oxazin-3-one

6-Bromo-7-chloro-4H-pyrido[3,2-b][1,4]oxazin-3-one (22 g, 83.7 mmole) and trans-2-phenylvinylboronic acid (17.33 g, 117 mmole) were dissolved in 1,4-dioxane (300 mL) and the solution was degassed with argon. (Ph₃P)₄Pd (1.9 g, 2 mole %) was added, followed by a solution of potassium hydrogen carbonate (21 g, 210 mmole) in H₂O (100 mL). The reaction was heated at reflux under argon overnight, then was cooled to room temperature and diluted with ethyl acetate (1 L). The solution was washed sequentially with H₂O and brine, dried (Na₂SO₄), and concentrated in vacuo. The residue was slurried with chloroform (120 mL), then diluted with diethyl ether (100 mL). The precipitated product was collected by filtration and washed with ether to provide the product (16.4 g, 68%) as an off-white solid. ¹H NMR (400 MHz, DMSO-d6) δ 4.71 (2H, s), 7.32-7.46 (3H, m), 7.54-7.74 (4H, m), 11.6 (1H, s).

### h) 7-Chloro-3-oxo-3,4-dihydro-2H-pyrido[3,2-b][1,4]oxazine-6-carboxaldehyde

7-Chloro-6-((E)-styryl)-4H-pyrido[3,2-b][1,4]oxazin-3-one (8.0 g, 27.9 mmole) was dissolved in a mixture of DMF (400 mL) and methanol (40 mL), and the solution was cooled to −78° C. Ozone was bubbled through the solution with stirring for 45 minutes, then the excess ozone was removed by bubbling oxygen through the solution for 30 min. Dimethylsulfide (21 mL, 279 mmole) was added to the solution, and the reaction was stirred at −78° C. for 3 hr, then at room temperature overnight. The solvent was removed in vacuo, and the residue was triturated with Et₂O (150 mL). The collected solid was washed with additional Et₂O and dried to afford a white solid (4 g, 68%). ¹H NMR (400 MHz, DMSO-d6) δ 4.86 (2H, m), 7.73 (1H, s); 10.05 (1H, s), 11.84 (1H, s).

### i) 7-chloro-3-oxo-3,4-dihydro-2H-1,4-benzoxazine-6-carboxylic acid

To a solution of 7-Chloro-3-oxo-3,4-dihydro-2H-pyrido[3,2-b][1,4]oxazine-6-carboxaldehyde (1.0 g, 4.71 mmol) in acetone-H2O (1:4, 5 mL) at 25° C. were added NaClO₂ (570 mg, 6.31 mmol) followed by H₂NSO₃H (594 mg, 6.12 mmol). After 2 h the solid precipitate was filtered affording the title compound as an off-white solid (840 mg, 71%): LCMS (ES) m/e 229 (M+H)⁺; ¹H NMR (CD₃OD, 400 MHz) δ 7.47 (s, 1H), 4.77 (s, 2H).

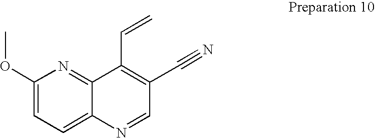

### Preparation of 4-ethenyl-6-(methyloxy)-1,5-naphthyridine-3-carbonitrile

### a) (2-[(6-Methoxypyridin-3-ylamino)methylene]malonic acid diethyl ester

A solution of 5-amino-2-methoxypyridine (Aldrich, 100 g, 0.806 mole) and diethyl ethoxymethylenemalonate (Aldrich, 163 mL, 0.806 mole) in EtOH (1 L) was heated at reflux for 4 hours, then cooled to RT. Concentration to dryness gave the title compound (238 g, quantitative).

### b) 6-Methoxy-4-oxo-1,4-dihydro-[1,5]naphthyridine-3-carboxylic acid ethyl ester

Dowtherm A (Fluka, 500 mL) was brought to boiling (250° C.) in a 2 L 3-neck flask fitted with a still-head and a reflux condenser. 2-[(6-Methoxypyridin-3-ylamino)methylene]malonic acid diethyl ester (100 g, 0.34 mole) was added portionwise over 5 min. The solution was heated at reflux for an additional 15 min, allowing some solvent to distill over. The resulting solution was cooled to RT and diluted with hexanes (750 mL). The mixture was cooled in ice for 1 hr, and the resulting brown precipitate was filtered off, washed with hexanes, and dried under vacuum to afford the title compound (61.72 g, 73%).

### c) 4-Bromo-6-methoxy-[1,5]naphthyridine-3-carboxylic acid ethyl ester

A suspension of 6-methoxy-4-oxo-1,4-dihydro-[1,5]naphthyridine-3-carboxylic acid ethyl ester (74.57 g, 300 mmole) in dry DMF (260 mL) under argon was stirred efficiently* in a water bath (to maintain approximately RT—may need slight ice-cooling on a large scale). Phosphorus tribromide (30.0 mL, 316 mmole) was added dropwise over 15 min and stirring was continued for an additional 30 min. Water (1 L) was added, followed by saturated sodium carbonate solution to pH 7. The solid was collected by suction filtration, washed with water and dried under vacuum over phosphorus pentoxide to give the title compound (83.56 g, 90%).

### d) 4-Bromo-6-methoxy-[1,5]naphthyridine-3-carboxylic acid

2 N NaOH (300 mL, 600 mmole) was added dropwise over 30 min to a stirred solution of 4-bromo-6-methoxy-[1,5]naphthyridine-3-carboxylic acid ethyl ester (83.56 g, 268 mmole) in THF (835 mL). Stirring was continued overnight, at which time LC/MS showed that the saponification was complete. 2 N HCl was added to pH 6 and the THF was removed in vacuo. 2 N HCl was added to pH 2, then water (250 mL) was added, and the mixture was cooled thoroughly in ice. The solid was collected by suction filtration, washed with water and dried (first using a rotary evaporator at 50° C. and then under high vacuum at 50° C. overnight) to give the title compound (76.7 g, slightly over quantitative). This material was used without further purification.

### (e) 4-chloro-6-(methyloxy)-1,5-naphthyridine-3-carboxamide

To a solution of 4-Bromo-6-methoxy-[1,5]naphthyridine-3-carboxylic acid ethyl ester (840 mg, 3.0 mmol) in toluene (10 mL) was added thionyl chloride (3 mL) as one portion under N₂ protection. After refluxing at 100° C. for 2 h, the mixture was concentrated and azeotropically dried with toluene to afford a yellow solid, which was dissolved in anhydrous DCM (3 mL). The resulting solution was cooled to 0° C. and treated with NH₃ solution (5 mL, 50% in water). After stirring at 0° C. for 30 min, the reaction mixture was warmed to 25° C. and stirred for 12 h. DCM was removed, and the solid was collected by suction filtration, washed with water and dried under vacuum over phosphorus pentoxide to give the title compound (648 mg, 91%).

### (f) 4-chloro-6-(methyloxy)-1,5-naphthyridine-3-carbonitrile

To a solution of 4-chloro-6-(methyloxy)-1,5-naphthyridine-3-carboxamide (647 mg, 2.7 mmol) in anhydrous DCM (2 mL) with triethyamine (2 mL) at 0° C. was added trifluororacetic anhydride (1 mL) slowly. The resulting solution was warmed to 25° C. and stirred for 1 h. The mixture was partitioned between CHCl₃ and H₂O and the aqueous layer was extracted several times with CHCl₃. The organic fractions were combined, concentrated and purified with column chromatography (silica, 0-25% ethyl acetate/hexane) affording the title compound as an off-white solid (540 mg, 91%): LC/MS (ES) m/e 220 (M+H)⁺.

### (g) 4-ethenyl-6-(methyloxy)-1,5-naphthyridine-3-carbonitrile

To a solution of 4-chloro-6-(methyloxy)-1,5-naphthyridine-3-carbonitrile (280 mg, 1.28 mmol), potassium carbonate (885 mg, 6.4 mmole), tetrakis-triphenylphosphine (30 mg, 0.026 mmole) in DME/H₂O (20 mL, 3:1) was added 2,4,6-trivinylcycloborane-pyridine complex (154 mg, 0.64 mmole). After stirring for 1 h at 90° C., another batch of tetrakis-triphenylphosphine (30 mg, 0.026 mmol) was added. After refluxing for another 1.5 h, the mixture contents were cooled to room temperature and extrated with diethyl ether. The ether fractions were combined, concentrated and purified by column chromatography (silica, 0-10% ehtyl acetate in hexane) to give the title compound as a light yellow solid (176 mg, 65%): LC/MS (ES) m/e 212 (M+H)⁺.

### EXAMPLE 1

### Preparation of N-(4-{2-[6-(methyloxy)-1,5-naphthyridin-4-yl]ethyl}-1-piperazinyl)-3-oxo-3,4-dihydro-2H-pyrido[3,2-b][1,4]thiazine-6-carboxamide

### (a) 2-(methyloxy)-8-[2-(4-nitroso-1-piperazinyl)ethyl]-1,5-naphthyridine

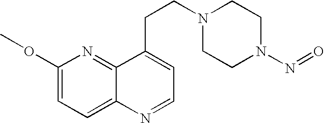

1-Nitrosopiperazine (402 mg, 3.50 mmol) [Prepared according to Vazques Tato, M. P.; Castedo, L.; Riguera, R. Chem. Lett. 1985, 623.] and 8-ethenyl-2-(methyloxy)-1,5-naphthyridine (592 mg, 3.17 mmol) were combined in DMF (1 mL) and stirred at 90° over 12 h. The solution was then concentrated and the residue purified via column chromatography (silica, 3% MeOH in DCM (1% NH₄OH)) yielding the title compound (400 mg, 38%) as an orange oil: LCMS (ES) m/e 302 (M+H)⁺.

### (b) 4-{2-[6-(methyloxy)-1,5-naphthyridin-4-yl]ethyl}-1-piperazinamine

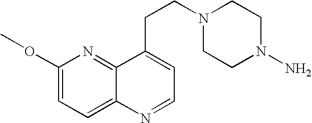

To a solution of 2-(methyloxy)-8-[2-(4-nitroso-1-piperazinyl)ethyl]-1,5-naphthyridine (366 mg, 1.22 mmol) in THF (12 mL) at 0° C. was added dropwise a solution of LAH (2.43 mL, 2.43 mmol, 1M in THF). The reaction mixture warmed to 25° C. over 12 h and was subsequently quenched by dropwise addition of a saturated solution of potassium sodium tartrate. The aqueous phase was extracted several times with DCM and the combined organic fractions were dried (Na₂SO₄) and concentrated at 25° C. yielding a yellow oil that was used without further purification: LCMS (ES) m/e 288 (M+H)⁺.

### (c) N-(4-{2-[6-(methyloxy)-1,5-naphthyridin-4-yl]ethyl}-1-piperazinyl)-3-oxo-3,4-dihydro-2H-pyrido[3,2-b][1,4]thiazine-6-carboxamide

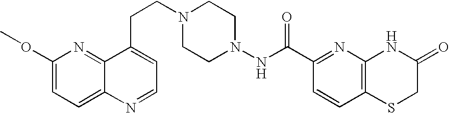

To a solution of crude 4-{2-[6-(methyloxy)-1,5-naphthyridin-4-yl]ethyl}-1-piperazinamine in DCM:DMF(12 ml, 4:1) was added EDC (227 mg, 1.46 mmol), HOBT (197 mg, 1.46 mmol) and 3-oxo-3,4-dihydro-2H-pyrido[3,2-b][1,4]thiazine-6-carboxylic acid (2.57 mg, 1.22 mmol). After 12 h at 25° C., the reaction was concentrated under reduced pressure and purified via trituration with MeOH affording the title compound as a off-white solid (200 mg, 40%): LCMS (ES) m/e 480 (M+H)+; ¹H NMR (CD₃OD, 400 MHz) δ 8.65 (d, J=4.8 Hz, 1H), 8.21 (d, J=9.1 Hz, 1H), 7.91 (d, J=7.9 Hz, 1H), 7.73 (d, J=7.9 Hz, 1H), 7.64 (d, J=4.6 Hz,1H), 7.24 (d, J=9.1 Hz,1H), 4.13 (s, 3H), 3.62 (s, 2H), 3.46-3.52 (m, 2H), 2.97-2.99 (m, 4H), 2.89-2.94 (m, 6H).

This material, as a solution in MeOH, was treated with an excess of 4M HCl in dioxane and evaporated to dryness to provide the dihydrochloride salt of the title compound.

### EXAMPLE 2

### Preparation of N-(4-{2-[6-(methyloxy)-1,5-naphthyridin-4-yl]ethyl}-1-piperazinyl)-3-oxo-3,4-dihydro-2H-pyrido[3,2-b][1,4]thiazine-6-sulfonamide

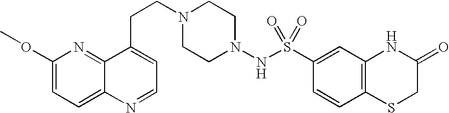

To a solution of crude 4-{2-[6-(methyloxy)-1,5-naphthyridin-4-yl]ethyl}-1-piperazinamine (350 mg, 1.22 mmol) in DCM (12 mL) was added DIPEA (425 μL, 2.44 mmol) and 3-oxo-3,4-dihydro-2H-pyrido[3,2-b][1,4]thiazine-6-sulfonyl chloride (320 mg, 1.22 mmol). After 2 h the solution was concentrated at room temperature and the residue purified by column chromatography (silica, 5% MeOH in DCM) affording the title compound as an off-white solid (37 mg, 6%): LCMS (ES) m/e 515 (M+H)+; ¹H NMR (CD₃OD, 400 MHz) δ 8.49 (d, J=4.6 Hz, 1H), 8.07 (d, J=9.1 Hz, 1H), 7.44 (d, J=4.6 Hz, 1H), 7.41 (bs, 3H), 7.10 (d, J=9.1 Hz, 1H), 3.97 (s, 3H), 3.42 (s, 2H), 3.25-3.29 (m, 2H), 2.66-2.70 (m, 2H), 2.49-2.56 (m, 8H).

This material, as a solution in MeOH, was treated with an excess of 4M HCl in dioxane and evaporated to dryness to provide the dihydrochloride salt of the title compound.

### EXAMPLE 3

### Preparation of N-(4-{2-[3-fluoro-6-(methyloxy)-1,5-naphthyridin-4-yl]ethyl}-1-piperazinyl)-3-oxo-3,4-dihydro-2H-pyrido[3,2-b][1,4]thiazine-6-carboxamide

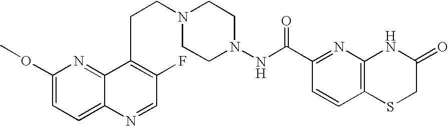

The title compound (500 mg, 51%) was prepared as an off-white solid according to Example 1, except substituting 8-ethenyl-7-fluoro-2-(methyloxy)-1,5-naphthyridine (620 mg, 3.65 mmol) for 8-ethenyl-2-(methyloxy)-1,5-naphthyridine: LCMS (ES) m/e 498 (M+H)⁺; ¹H NMR (CD₃OD, 400 MHz) δ 8.67 (s, 1H), 8.23 (d, J=9.1 Hz, 1H), 7.91 (d, J=8.1 Hz, 1H), 7.73 (d, J=9.1 Hz, 1H), 7.20 (d, J=9.1 Hz, 1H), 4.14 (s, 3H), 3.62 (s, 2H), 3.48-3.51 (m, 2H), 2.94-2.97 (m, 4H), 2.85-2.87 (m, 6H).

This material, as a solution in MeOH, was treated with an excess of 4M HCl in dioxane and evaporated to dryness to provide the dihydrochloride salt of the title compound.

### EXAMPLE 4

### Preparation of N-(4-{(2S)-2-hydroxy-2-[6-(methyloxy)-1,5-naphthyridin-4-yl]ethyl}-1-piperazinyl)-3-oxo-3,4-dihydro-2H-pyridro[3,2-b][1,4]thiazine-6-carboxamide

### (a) (1S)-1-[6-(methyloxy)-1,5-naphthyridin-4-yl]-2-(4-nitroso-1-piperazinyl)ethanol

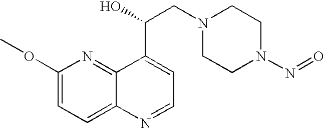

A solution of 2-(methyloxy)-8-(2-oxiranyl)-1,5-naphthyridine (712 mg, 3.51 mmol) and 1-nitrosopiperazine (404 mg, 3.51 mmol) [Prepared according to Vazques Tato, M. P.; Castedo, L.; Riguera, R. Chem. Lett. 1985, 623.] in DMF (1 mL) were heated to 90° C. After 12 h, the resulting solution was concentrated and purified via column chromatography (silica, 2% MeOH in DCM (1% NH₄OH) yielding the title compound as an orange oil (1.05 g, 94%): LCMS (ES) m/e 318 (M+H)⁺.

### (b) N-(4-{(2S)-2-hydroxy-2-[6-(methyloxy)-1,5-naphthyridin-4-yl]ethyl}-1-piperazinyl)-3-oxo-3,4-dihydro-2H-pyrido[3,2-b][1,4]thiazine-6-carboxamide

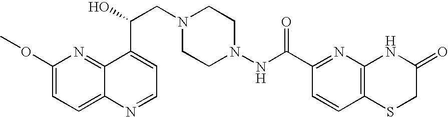

The title compound (50 mg, 19%) was prepared as a yellow solid according to Example 1, except substituting (1S)-1-[6-(methyloxy)-1,5-naphthyridin-4-yl]-2-(4-nitroso-1-piperazinyl)ethanol (1.05 g, 3.31 mmol) for 2-(methyloxy)-8-[2-(4-nitroso-1-piperazinyl)ethyl]-1,5-naphthyridine: LCMS (ES) m/e 496 (M+H)⁺; ¹H NMR (CD₃OD, 400 MHz) δ 8.74 (d, J=4.6 Hz, 1H), 8.21 (d, J=9.1 Hz, 1H), 7.87-7.90 (m, 2H), 7.71 (d, J=7.9 Hz, 1H), 7.23 (d, J=9.1 Hz, 1H), 5.97 (dd, J=2.5, 9.2 Hz, 1H), 4.10 (s, 3H), 3.62 (s, 2H), 2.95-3.01 (m, 6H), 2.81-2.93 (m, 2H), 2.63-2.69 (m, 2H).

This material, as a solution in MeOH, was treated with an excess of 4M HCl in dioxane and evaporated to dryness to provide the dihydrochloride salt of the title compound.

### EXAMPLE 5

### Preparation of N-(4-{2-[3-chloro-6-(methyloxy)-1,5-naphthyridin-4-yl]ethyl}-1-piperazinyl)-3-oxo-3,4-dihydro-2H-pyrido[3,2-b][1,4]thiazine-6-carboxamide

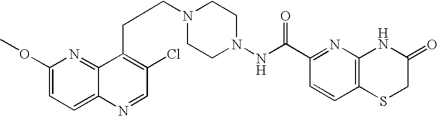

The title compound (700 mg, 80%) was prepared as an off-white solid according to Example 1, except substituting 7-chloro-8-ethenyl-2-(methyloxy)-1,5-naphthyridine (1.0 g, 4.55 mmol) for 8-ethenyl-2-(methyloxy)-1,5-naphthyridine: LCMS (ES) m/e 515 (M+H)⁺; ¹H NMR (CD₃OD, 400 MHz) δ 8.71 (s, 1H), 8.22 (d, J=9.1 Hz, 1H), 7.91 (d, J=7.9 Hz, 1H), 7.73 (d, J=7.9 Hz, 1H), 7.25 (d, J=9.1 Hz, 1H), 4.14 (s, 3H), 3.63-3.68 (m, 2H), 3.62 (s, 2H), 2.98-3.01 (m, 4H), 2.81-2.86 (m, 6H).

This material, as a solution in MeOH, was treated with an excess of 4M HCl in dioxane and evaporated to dryness to provide the dihydrochloride salt of the title compound.

### EXAMPLE 6

### Preparation of N-(4-{2-[3-chloro-6-(methyloxy)-1,5-naphthyridin-4-yl]ethyl}-1-piperazinyl)-3-oxo-3,4-dihydro-2H-pyrido[3,2-b][1,4]oxazine-6-carboxamide

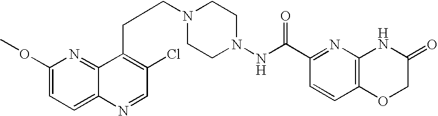

The title compound (700 mg, 82%) was prepared as an off-white solid according to Example 1, except substituting 7-chloro-8-ethenyl-2-(methyloxy)-1,5-naphthyridine (1.0 g, 4.55 mmol) for 8-ethenyl-2-(methyloxy)-1,5-naphthyridine and 3-oxo-3,4-dihydro-2H-pyrido[3,2-b][1,4]oxazine-6-carboxylic acid (332 mg, 1.71 mmol) for 3-oxo-3,4-dihydro-2H-pyrido[3,2-b][1,4]thiazine-6-carboxylic acid: LCMS (ES) m/e 498 (M+H)⁺; ¹H NMR (CD₃OD, 400 MHz) δ 8.71 (s, 1H), 8.22 (d, J=9.1 Hz, 1H), 7.75 (d, J=8.2 Hz, 1H), 7.43 (d, J=8.1 Hz, 1H), 7.25 (d, J=9.1 Hz, 1H), 4.77 (s, 2H), 4.14 (s, 3H), 3.63-3.68 (m, 2H), 2.97-3.00 (m, 4H), 2.83-2.86 (m, 6H).

This material, as a solution in MeOH, was treated with an excess of 4M HCl in dioxane and evaporated to dryness to provide the dihydrochloride salt of the title compound.

### EXAMPLE 7

### Preparation of N-methyl-N-(4-{2-[6-(methyloxy)-1,5-naphthyridin-4-yl]ethyl}-1-piperazinyl)-3-oxo-3,4-dihydro-2H-pyrido[3,2-b][1,4]thiazine-6-carboxamide

### (a) 1,1-dimethylethyl 4-{2-[6-(methyloxy)-1,5-naphthyridin-4-yl]ethyl}-1-piperazinecarboxylate

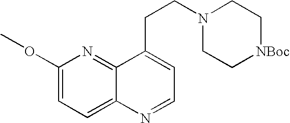

The title compound (2.1 g, 53%) was prepared as a yellow oil according to Example 1, except substituting 1,1-dimethylethyl 1-piperazinecarboxylate (2 g, 10.7 mmol) for 1-nitrosopiperazine: LCMS (ES) m/e 317 (M-tBu)⁺.

### (b) 2-(methyloxy)-8-[2-(1-piperazinyl)ethyl]-1,5-naphthyridine

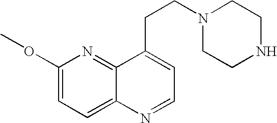

To a solution of 1,1-dimethylethyl 4-{2-[6-(methyloxy)-1,5-naphthyridin-4-yl]ethyl}-1-piperazinecarboxylate (2.0 g, 5.41 mmol) in MeOH (27 mL) at 25° C. was added an HCl solution (8.0 mL, 32.44 mmol, 4M HCl in dioxane) dropwise. After 12 h, the solution was concentrated and the residue neutralized with excess DIPEA. The free base was passed through a silica plug (5% MeOH in DCM (1% NH₄OH)) affording the title compound as a off-white solid (1.3 g, 92%): LCMS (ES) m/e 273 (M+H)⁺.

### (c) 2-(methyloxy)-8-[2-(4-nitroso-1-piperazinyl)ethyl]-1,5-naphthyridine

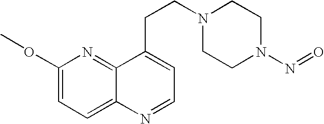

To a solution of 2-(methyloxy)-8-[2-(1-piperazinyl)ethyl]-1,5-naphthyridine (500 mg, 1.84 mmol) in DCM (18 mL) was added, in one portion, Diazald® (788 mg, 3.68 mmol). After stirring at reflux for 12 h, the solution was cooled, concentrated and purified by column chromatography (silica, 1% MeOH in DCM (1% NH₄OH)) affording the title compound as a orange oil (480 mg, 80% brsm), which was identical to that reported in Example 1.

### (d) N-methyl-4-{2-[6-(methyloxy)-1,5-naphthyridin-4-yl]ethyl}-1-piperazinamine

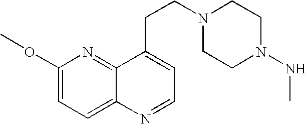

To a solution of 4-{2-[6-(methyloxy)-1,5-naphthyridin-4-yl]ethyl}-1-piperazinamine (346 mg, 1.21 mmol) in MeOH (1.0 mL) at 25° C. was added formaldehyde (37 wt % in H₂O, 90 μL, 1.21 mmol). After 2 h, the solution was concentrated and the residue was used directly without further purification: LCMS (ES) m/e 300 (M+H)⁺.

To the crude imine (326 mg, 1.09 mmol) in EtOH (6 mL) at 0° C. was added dropwise AcOH (98 μL) followed by portion-wise addition of NaBH₄ (412 mg, 10.9 mmol). After 12 h warming to 25° C., the solution was concentrated and the residue partitioned between DCM-H₂O. The combined organic fractions were dried (Na₂SO₄) and the crude residue was purified via column chromatography (silica, 1-2% MeOH in DCM (1% NH₄OH)) yielding the title compound as a yellow oil (125 mg, 38%): LCMS (ES) m/e 302 (M+H)⁺.

### (e) N-methyl-N-(4-{2-[6-(methyloxy)-1,5-naphthyridin-4-yl]ethyl}-1-piperazinyl)-3-oxo-3,4-dihydro-2H-pyrido[3,2-b][1,4]thiazine-6-carboxamide

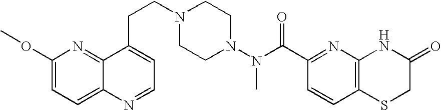

The title compound (120 mg, 59%) was prepared as a yellow solid according to Example 1, except substituting N-methyl-4-{2-[6-(methyloxy)-1,5-naphthyridin-4-yl]ethyl}-1-piperazinamine (125 mg, 0.415 mmol) for 4-{2-[6-(methyloxy)-1,5-naphthyridin-4-yl]ethyl}-1-piperazinamine: LCMS (ES) m/e 494 (M+H)⁺; ¹H NMR (CD₃OD, 400 MHz) δ 8.57 (d, J=4.4 Hz, 1H), 8.13 (d, J=9.0 Hz, 1H), 7.55 (d, J=7.6 Hz, 1H), 7.26 (d, J=4.4 Hz, 1H), 7.00 (9.0 Hz, 1H), 6.92 (d, J=7.5 Hz, 1H), 3.94 (s, 3H), 3.43 (s, 2H), 3.09-3.19 (m, 2H), 2.99 (s, 3H), 2.70-2.88 (m, 4H), 2.59-2.68 (m, 4H), 1.84-1.89 (m, 2H).

This material, as a solution in MeOH, was treated with an excess of 4M HCl in dioxane and evaporated to dryness to provide the dihydrochloride salt of the title compound.

### EXAMPLE 8

### Preparation of N-(4-{2-[3-fluoro-6-(methyloxy)-1,5-naphthyridin-4-yl]ethyl}-1-piperazinyl)-3-oxo-3,4-dihydro-2H-pyrido[3,2-b][1,4]oxazine-6-carboxamide

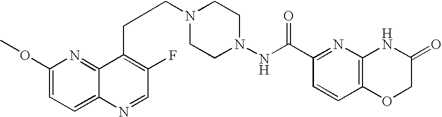

The title compound (140 mg, 88%) was prepared as an off-white solid according to Example 7, except substituting 8-ethenyl-7-fluoro-2-(methyloxy)-1,5-naphthyridine (100 mg, 0.33 mmol) for 8-ethenyl-2-(methyloxy)-1,5-naphthyridine and 3-oxo-3,4-dihydro-2H-pyrido[3,2-b][1,4]oxazine-6-carboxylic acid (64 mg, 0.35 mmol) for 3-oxo-3,4-dihydro-2H-pyrido[3,2-b][1,4]thiazine-6-carboxylic acid: LCMS (ES) m/e 482 (M+H)⁺; ¹H NMR (CDCl₃, 400 MHz) δ 7.10 (s, 1H), 6.66 (d, J=9.2 Hz, 1H), 6.20 (d, J=8.4 Hz, 1H), 5.85 (d, J=8.4 Hz, 1H), 5.64 (d, J=9.2 Hz, 1H), 3.20 (s, 2H), 2.58 (s, 3H), 1.97-1.91 (m, 2H), 1.42-1.36 (m, 4H), 1.34-1.24 (m, 6H).

This material, as a solution in MeOH, was treated with an excess of 4M HCl in dioxane and evaporated to dryness to provide the dihydrochloride salt of the title compound.

### EXAMPLE 9

### Preparation of N-(4-{2-[3-fluoro-6-(methyloxy)-1,5-naphthyridin-4-yl]ethyl}-1-piperazinyl)-2,3-dihydro[1,4]dioxino[2,3-c]pyridine-7-carboxamide

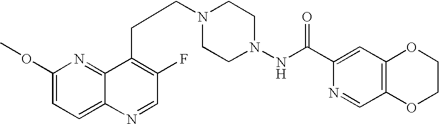

The title compound (20 mg, 24%) was prepared as an off-white solid according to Example 7, except substituting 8-ethenyl-7-fluoro-2-(methyloxy)-1,5-naphthyridine (55 mg, 0.18 mmol) for 8-ethenyl-2-(methyloxy)-1,5-naphthyridine and 2,3-dihydro[1,4]dioxino[2,3-c]pyridine-7-carboxylic acid (42 mg, 0.23 mmol) for 3-oxo-3,4-dihydro-2H-pyrido[3,2-b][1,4]thiazine-6-carboxylic acid: LCMS (ES) m/e 469 (M+H)⁺; ¹H NMR (CDCl₃, 400 MHz) δ 8.61 (s, 1H), 8.50 (s, 1H), 8.14 (d, J=9.2 Hz, 1H), 8.05 (s, 1H), 7.73 (s, 1H), 7.07 (d, J=9.2 Hz, 1H), 4.37-4.32 (m, 4H), 4.08 (s, 3H), 3.38-3.43 (m, 2H), 3.0-2.94 (m, 4H), 2.87-2.78 (m, 6H).

This material, as a solution in MeOH, was treated with an excess of 4M HCl in dioxane and evaporated to dryness to provide the dihydrochloride salt of the title compound.

### EXAMPLE 10

### Preparation of 7-chloro-N-(4-{2-[3-fluoro-6-(methyloxy)-1,5-naphthyridin-4-yl]ethyl}-1-piperazinyl)-2,3-dihydro[1,4]dioxino[2,3-b]pyridine-6-carboxamide

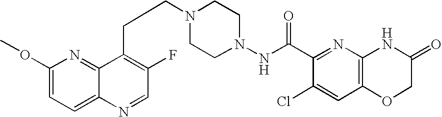

The title compound (33 mg, 36%) was prepared as an off-white solid according to Example 7, except substituting 8-ethenyl-7-fluoro-2-(methyloxy)-1,5-naphthyridine (55 mg, 0.18 mmol) for 8-ethenyl-2-(methyloxy)-1,5-naphthyridine and 7-chloro-2,3-dihydro[1,4]dioxino[2,3-b]pyridine-6-carboxylic acid (53 mg, 0.23 mmol) for 3-oxo-3,4-dihydro-2H-pyrido[3,2-b][1,4]thiazine-6-carboxylic acid: LCMS (ES) m/e 516 (M+H)⁺; ¹H NMR (CDCl₃, 400 MHz) δ 8.61 (s, 1H), 8.17 (d, J=9.2 Hz, 1H), 7.92 (s, 1H), 7.35 (s, 1H), 7.07 (d, J=9.2 Hz, 1H), 4.73 (s, 2H), 4.07 (s, 3H), 3.75-3.81 (bs, 1H), 3.40-3.44 (m, 2H), 3.03-2.98 (m, 4H), 2.91-2.83 (m, 6H).

This material, as a solution in MeOH, was treated with an excess of 4M HCl in dioxane and evaporated to dryness to provide the dihydrochloride salt of the title compound.

### EXAMPLE 11

### Preparation of N-((1S,4S)-5-{2-[3-fluoro-6-(methyloxy)-1,5-naphthyridin-4-yl]ethyl}-2,5-diazabicyclo[2.2.1]hept-2-yl)-3-oxo-3,4-dihydro-2H-pyrido[3,2-b][1,4]thiazine-6-carboxamide

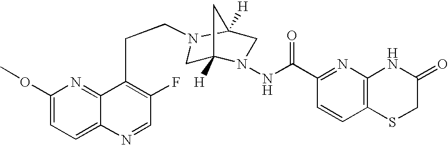

The title compound (33 mg, 36%) was prepared as a yellow solid according to Example 7, except substituting 8-ethenyl-7-fluoro-2-(methyloxy)-1,5-naphthyridine (55 mg, 0.18 mmol) for 8-ethenyl-2-(methyloxy)-1,5-naphthyridine and 1,1-dimethylethyl (1S,4S)-2,5-diazabicyclo[2.2.1]heptane-2-carboxylate (250 mg, 1.27 mmol) for 1,1-dimethylethyl 1-piperazinecarboxylate: LCMS (ES) m/e 510 (M+H)⁺; ¹H NMR (CD₃OD, 400 MHz) δ 8.54 (s, 1H), 8.07 (d, J=9.1 Hz, 1H), 7.75 (d, J=7.9 Hz, 1H), 7.55 (d, J=7.9 Hz, 1H), 9.1 (d, J=9.1 Hz, 1H), 3.99 (s, 3H), 3.53 (d, J=8.1 Hz, 2H), 3.49 (s, 2H), 3.31 (dd, J=7.4, 8.0 Hz, 2H), 3.10 (d, J=11.1 Hz, 1H), 2.92-3.00 (m, 3H), 2.77-2.90 (m, 2H), 1.87 (d, J=10.2 Hz, 1H), 1.78 (d, J=10.5 Hz, 1H).

This material, as a solution in MeOH, was treated with an excess of 4M HCl in dioxane and evaporated to dryness to provide the dihydrochloride salt of the title compound.

### EXAMPLE 12

### Preparation of N′-methyl-N′-(1-{2-[6-(methyloxy)-1,5-naphthyridin-4-yl]ethyl}-3-pyrrolidinyl)-3-oxo-3,4-dihydro-2H-pyrido[3,2-b][1,4]thiazine-6-carboxamide

### (a) 1,1-dimethylethyl methyl(1-{2-[6-(methyloxy)-1,5-naphthyridin-4-yl]ethyl}-3-pyrrolidinyl)carbamate

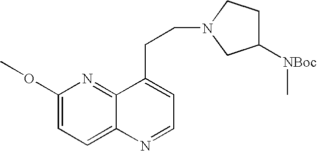

A solution of 1,1-dimethylethyl methyl(3-pyrrolidinyl)carbamate (1.3 mL, 6.45 mmol) and 8-ethenyl-2-(methyloxy)-1,5-naphthyridine (1.2 g, 6.45 mmol) in DMF (1 mL) was heated to 90° C. After 12 h, the solution was cooled, concentrated and the residue purified via column chromatography (silica, 0-1% MeOH in DCM (1% NH₄OH)) yielding the title compound as a yellow oil (2.5 g, quant.): LCMS (ES) m/e 386 (M+H)⁺.

### (b) N-methyl-1-{2-[6-(methyloxy)-1,5-naphthyridin-4-yl]ethyl}-3-pyrrolidinamine

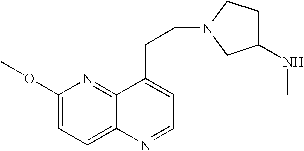

A solution of 1,1-dimethylethyl methyl(1-{2-[6-(methyloxy)-1,5-naphthyridin-4-yl]ethyl}-3-pyrrolidinyl)carbamate (2.5 g, 6.45 mmol) in MeOH (32 mL) was treated dropwise with a solution of HCl (8 mL, 32.2 mmol, 4M solution in dioxane). After 12 h, the solution was concentrated and neutralized with excess DIPEA. The residue was purified through a small plug of silica (3% MeOH in DCM (1% NH₄OH)) yielding the title compound as a yellow oil (1.7 g, 92%): LCMS (ES) m/e 287 (M+H)⁺.

### (c) N-methyl-1-{2-[6-(methyloxy)-1,5-naphthyridin-4-yl]ethyl}-N-nitroso-3-pyrrolidinamine

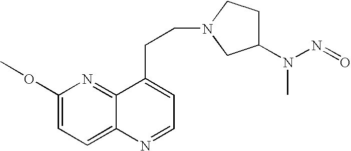

To a solution of N-methyl-1-{2-[6-(methyloxy)-1,5-naphthyridin-4-yl]ethyl}-3-pyrrolidinamine (1.7 g, 5.94 mmol) in DCM (59 mL) was added, in one portion, Diazald® (3.2 g, 14.86 mmol). After stirring at reflux for 12 h, the solution was cooled, concentrated and purified by column chromatography (silica, 1% MeOH in DCM (1% NH₄OH)) affording the title compound as an orange oil (1.1 g, 70% brsm): LCMS (ES) m/e 316 (M+H)⁺.

### (d) 8-{2-[3-(1-methylhydrazino)-1-pyrrolidinyl]ethyl}-2-(methyloxy)-1,5-naphthyridine

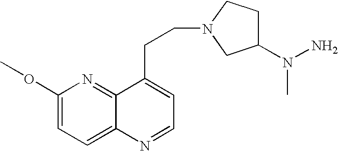

To a solution of N-methyl-1-{2-[6-(methyloxy)-1,5-naphthyridin-4-yl]ethyl}-N-nitroso-3-pyrrolidinamine (100 mg, 0.317 mmol) in THF (3.2 mL) at 0° C. was added dropwise a solution of LAH (1.3 mL, 1.27 mmol, 1M in THF). The reaction mixture warmed to 25° C. over 12 h and was subsequently quenched by dropwise addition of a saturated solution of potassium sodium tartrate. The aqueous phase was extracted several times with DCM and the combined organic fractions were dried (Na₂SO₄) and concentrated at 25° C. yielding a yellow oil that was used without further purification: LCMS (ES) m/e 302 (M+H)⁺.

### (e) N′-methyl-N′-(1-{2-[6-(methyloxy)-1,5-naphthyridin-4-yl]ethyl}-3-pyrrolidinyl)-3-oxo-3,4-dihydro-2H-pyrido[3,2-b][1,4]thiazine-6-carbohydrazide

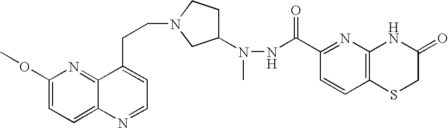

To a solution of 8-{2-[3-(1-methylhydrazino)-1-pyrrolidinyl]ethyl}-2-(methyloxy)-1,5-naphthyridine (73 mg, 0.243 mmol) in DCM-DMF (4:1, 2 mL) were added 3-oxo-3,4-dihydro-2H-pyrido[3,2-b][1,4]thiazine-6-carboxylic acid (51 mg, 0.243 mmol), EDC (45 mg, 0.291 mmol) and HOBT (39 mg, 0.291 mmol). After 12 h, the solution was concentrated and the residue purified via column chromatography (silica, 1-3% MeOH in DCM (1% NH₄OH)) yielding the title compound as a yellow foam (32 mg, 27%): LCMS (ES) m/e 494 (M+H)⁺; ¹H NMR (CD₃OD, 400 MHz) δ 8.45 (d, J=4.5 Hz, 1H), 8.02 (d, J=9.0 Hz, 1H), 7.78 (d, J=7.9 Hz, 1H), 7.59 (d, J=7.9 Hz, 1H), 7.43 (d, J=4.5 Hz, 1H), 7.05 (d, J=9.0 Hz, 1H), 3.90 (s, 3H), 3.50 (s, 2H), 3.43-3.47 (m, 1H), 3.24-3.29 (m, 2H), 2.93-3.04 (m, 1H), 2.70-2.89 (m, 3H), 2.55-2.69 (m, 1H), 2.52 (s, 3H), 2.43-2.49 (m,1H), 1.91-1.98 (m, 1H), 1.74-1.81 (m,1H).

This material, as a solution in MeOH, was treated with an excess of 4M HCl in dioxane and evaporated to dryness to provide the dihydrochloride salt of the title compound.

### EXAMPLE 13

### Preparation of N-(4-hydroxy-4-{2-[6-(methyloxy)-1,5-naphthyridin-4-yl]ethyl}-1-piperidinyl)-3-oxo-3,4-dihydro-2H-pyrido[3,2-b][1,4]thiazine-6-carboxamide

### (a) 1,1-dimethylethyl4-ethynyl-4-hydroxy-1-piperidinecarboxylate

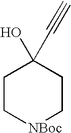

To a solution of 1,1-dimethylethyl 4-hydroxy-4-[(trimethylsilyl)ethynyl]-1-piperidinecarboxylate (1.0 g, 3.37 mmol) [prepared according to J. Med. Chem. 1999, 42, 12, 2087.] in THF (34 mL) at 0° C. was added TBAF (4 mL, 4.04 mmol, 1M in THF). After warming to 25° C. over 12 h, the solution was partitioned between H₂O-DCM. The aqueous phase was washed several times with DCM and the combined organic fractions were dried (Na₂SO₄), concentrated and the residue purified via column chromatography (silica, 2% MeOH in DCM (1% NH₄OH)) affording the title compound as a white solid (760 mg, quant.): LCMS (ES) m/e 226 (M+H)⁺.

### (b) 1,1-dimethylethyl 4-hydroxy-4-{2-[6-(methyloxy)-1,5-naphthyridin-4-yl]ethynyl}-1-piperidinecarboxylate

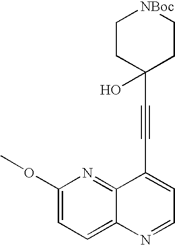

To a solution of 6-(methyloxy)-1,5-naphthyridin-4-yl trifluoromethanesulfonate (342 mg, 1.11 mmol) in DMF-Et₃N (1.6:1, 4 mL) were added 1,1-dimethylethyl 4-ethynyl-4-hydroxy-1-piperidinecarboxylate (300 mg, 1.33 mmol), CuI (11 mg, 55.5 μmol) and Pd(PPh₃)₂Cl₂ (78 mg, 0.11 mmol). The reaction contents were heated for 20 min at 120° C. in a microwave reactor and then partitioned between H₂O-DCM. The aqueous phase was extracted several times with DCM and the combined organic fractions were dried (Na₂SO₄), concentrated and purified via column chromatography (silica, 0-2% MeOH in DCM (1% NH₄OH)) yielding the title compound as a yellow oil (550 mg, quant.): LCMS (ES) m/e 384 (M+H)⁺.

### (c) 1,1-dimethylethyl 4-hydroxy-4-{2-[6-(methyloxy)-1,5-naphthyridin-4-yl]ethyl}-1-piperidinecarboxylate

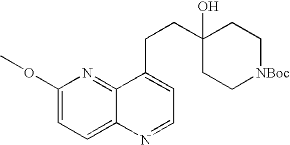

A solution of 1,1-dimethylethyl 4-hydroxy-4-{[6-(methyloxy)-1,5-naphthyridin-4-yl]ethynyl}-1-piperidinecarboxylate (550 mg, 1.43 mmol) and 10% Pd—C (40 wt %, 220 mg) in EtOH (14 mL) were hydrogenated at 50 psi using a Parr Shaker. After 12 h, the solution was filtered through Celite, concentrated and purified via column chromatography (silica, 2% MeOH in DCM (1% NH₄OH)) yielding the title compound as a clear oil (450 mg, 57%): LCMS (ES) m/e 388 (M+H)⁺.

### (d) Preparation of 4-{2-[6-(methyloxy)-1,5-naphthyridin-4-yl]ethyl}-4-piperidinol

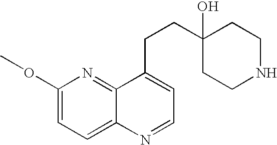

To a solution of 1,1-dimethylethyl 4-hydroxy-4-{2-[6-(methyloxy)-1,5-naphthyridin-4-yl]ethyl}-1-piperidinecarboxylate (450 mg, 1.16 mmol) in MeOH (6 mL) at 25° C. was added dropwise a solution of HCl in dioxane (872 μL, 4M in dioxane). After 8 h, the solution was concentrated and the residue was used directly without further purification: LCMS (ES) m/e 288 (M+H)⁺.

### (e) 4-{2-[6-(methyloxy)-1,5-naphthyridin-4-yl]ethyl}-1-nitroso-4-piperidinol

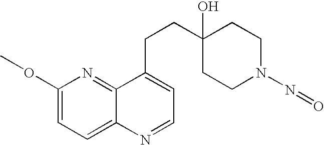

To a solution of crude 4-{2-[6-(methyloxy)-1,5-naphthyridin-4-yl]ethyl}-4-piperidinol (1.16 mmol) and potassium nitrosodisulfonate (3.9 g, 14.5 mmol) in 2% Na₂CO₃ (58 mL) and pyridine (8.0 mL) was added NH₂OH.HCl (582 mg, 8.57 mmol) in H₂O (5 mL). After 30 min, the solution was partitioned between H₂O-DCM. The aqueous phase was extracted several times with DCM and the combined organic fractions were dried (Na₂SO₄), concentrated and the residue was used directly without further purification: LCMS (ES) m/e 317 (M+H)⁺.

### (f) 1-amino-4-{2-[6-(methyloxy)-1,5-naphthyridin-4-yl]ethyl}-4-piperidinol

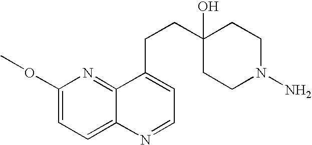

To a solution of 4-{2-[6-(methyloxy)-1,5-naphthyridin-4-yl]ethyl}-1-nitroso-4-piperidinol (215 mg, 0.68 mmol) in THF (7 mL) at 0° C. was added dropwise a solution of LAH (2.2 mL, 1M in THF). After warming to 25° C. over 12 h, the solution was quenched by dropwise addition of a saturated aqueous solution of sodium potassium tartrate. This mixture was then partitioned between H₂O-DCM and the aqueous phase was washed several times with DCM. The combined organic fractions were dried (Na₂SO₄), concentrated and used directly without further purification: LCMS (ES) m/e 302 (M+H)⁺.

### (g) N-(4-hydroxy-4-{2-[6-(methyloxy)-1,5-naphthyridin-4-yl]ethyl}-1-piperidinyl)-3-oxo-3,4-dihydro-2H-pyrido[3,2-b][1,4]thiazine-6-carboxamide

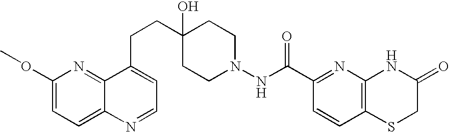

To a solution of 1-amino-4-{2-[6-(methyloxy)-1,5-naphthyridin-4-yl]ethyl}-4-piperidinol (143 mg, 0.476 mmol) in DCM-DMF (4:1, 5 mL) were added 3-oxo-3,4-dihydro-2H-pyrido[3,2-b][1,4]thiazine-6-carboxylic acid (111 mg, 0.524 mmol), EDC (104 mg, 0.667 mmol) and HOBT (90 mg, 0.667 mmol). After 12 h, the solution was concentrated and the residue purified via column chromatography (silica, 3% MeOH in DCM (1% NH₄OH)) yielding the title compound as a yellow foam (89 mg, 38%): LCMS (ES) m/e 495 (M+H)⁺; ¹H NMR (CD₃OD, 400 MHz) δ 8.57 (d, J=4.4 Hz, 1H), 8.12 (d, J=9.0 Hz, 1H), 7.80 (d, J=7.8 Hz, 1H), 7.66 (d, J=7.8 Hz, 1H), 7.50 (d, J=4.5 Hz, 1H), 7.14 (d, J=9.0 Hz, 1H), 4.08 (s, 3H), 3.59 (s, 2H), 3.24-3.33 (m, 2H), 2.99-3.09 (m, 2H), 2.94-2.96 (m, 2H), 1.69-1.89 (m, 6H).

This material, as a solution in MeOH, was treated with an excess of 4M HCl in dioxane and evaporated to dryness to provide the dihydrochloride salt of the title compound.

### EXAMPLE 14

### Preparation of N-(4-{2-[3-fluoro-6-(methyloxy)-1,5-naphthyridin-4-yl]ethyl}-4-hydroxy-1-piperidinyl)-3-oxo-3,4-dihydro-2H-pyrido[3,2-b][1,4]thiazine-6-carboxamide

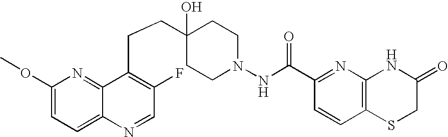

The title compound (36 mg, 10%) was prepared according to Example 13, except substituting 8-bromo-7-fluoro-2-(methyloxy)-1,5-naphthyridine (300 mg, 1.17 mmol) for 6-(methyloxy)-1,5-naphthyridin-4-yl trifluoromethanesulfonate: LCMS (ES) m/e 513 (M+H)⁺; ¹H NMR (CD₃OD, 400 MHz) δ 8.68 (s, 1H), 8.21 (d, J=9.0 Hz, 1H), 7.93 (d, J=7.8 Hz, 1H), 7.76 (d, J=7.9 Hz, 1H), 7.20 (d, J=9.1 Hz, 1H), 4.15 (s, 3H), 3.63 (s, 2H), 3.44-3.49 (m, 2H), 3.33-3.39 (m, 4H), 2.08-2.13 (m, 2H), 1.90-2.05 (m, 4H).

This material, as a solution in MeOH, was treated with an excess of 4M HCl in dioxane and evaporated to dryness to provide the dihydrochloride salt of the title compound.

### EXAMPLE 15

### Preparation of 5-fluoro-N-(4-{2-[3-fluoro-6-(methyloxy)-1,5-naphthyridin-4-yl]ethyl}-1-piperazinyl)-1H-indole-2-carboxamide

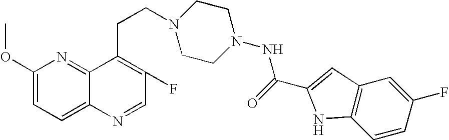

The title compound (22.4 mg, 21%) was prepared as a yellow solid according to Example 7, except substituting 8-ethenyl-7-fluoro-2-(methyloxy)-1,5-naphthyridine (300 mg, 1.47 mmol) for 8-ethenyl-2-(methyloxy)-1,5-naphthyridine and 5-fluoro-1H-indole-2-carboxylic acid (54 mg, 0.30 mmol) for 3-oxo-3,4-dihydro-2H-pyrido[3,2-b][1,4]thiazine-6-carboxylic acid: LCMS (ES) m/e 467 (M+H)⁺; ¹H NMR (CD₃OD, 400 MHz)δ 8.63 (s, 1H), 8.19 (d, J=9 Hz, 1H), 7.34-7.37 (m, 2H), 7.07-7.10 (m, 2H), 6.75-6.9 (m,1H), 4.08 (s, 3H), 3.41-3.49 (m, 2H), 3.2-3.3 (m,1H), 3.05-3.2 (m,1H), 2.95-3.03 (m, 2H), 2.82-2.91 (m, 4H), 2.62-2.75 (m, 1H), 2.49-2.61 (m, 1H).

This material, as a solution in MeOH, was treated with an excess of 4M HCl in dioxane and evaporated to dryness to provide the dihydrochloride salt of the title compound.

### EXAMPLE 16

### Preparation of N-(4-{2-[3-fluoro-6-(methyloxy)-1,5-naphthyridin-4-yl]ethyl}-1-piperazinyl)-3,4-dihydro-2H-1,5-benzodioxepin-7-carboxamide

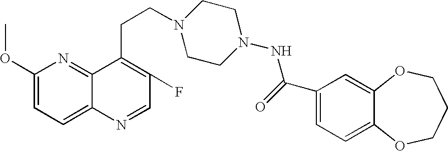

The title compound (13.8 mg, 12%) was prepared as a yellow solid according to Example 7, except substituting 8-ethenyl-7-fluoro-2-(methyloxy)-1,5-naphthyridine (300 mg, 1.47 mmol) for 8-ethenyl-2-(methyloxy)-1,5-naphthyridine and 3,4-dihydro-2H-1,5-benzodioxepin-7-carboxylic acid (59 mg, 0.30 mmol) for 3-oxo-3,4-dihydro-2H-pyrido[3,2-b][1,4]thiazine-6-carboxylic acid: LCMS (ES) m/e 482 (M+H)⁺; ¹H NMR (CDCl₃, 400 MHz) δ 8.63 (s, 1H), 8.19 (d, J=9.0 Hz, 1H), 7.31-7.38 (m, 2H), 7.07 (d, J=Hz, 1H), 6.95 (d, J=Hz, 1H), 6.58 (s, 1H), 4.22-4.32 (m, 4H), 4.05(s, 3H), 3.36-3.48 (m, 2H), 2.88-2.97 (m, 3H), 2.75-2.86 (m, 6H), 2.17-2.26 (m, 2H).

This material, as a solution in MeOH, was treated with an excess of 4M HCl in dioxane and evaporated to dryness to provide the dihydrochloride salt of the title compound.

### EXAMPLE 17

### Preparation of N-(4-{2-[3-fluoro-6-(methyloxy)-1,5-naphthyridin-4-yl]ethyl}-1-piperazinyl)-2-(1H-indol-3-yl)-2-oxoacetamide

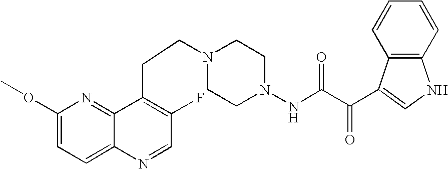

The title compound (30 mg, 27%) was prepared as a white solid according to Example 7, except substituting 8-ethenyl-7-fluoro-2-(methyloxy)-1,5-naphthyridine (300 mg, 1.47 mmol) for 8-ethenyl-2-(methyloxy)-1,5-naphthyridine and 1H-indol-3-yl(oxo)acetic acid (57 mg, 0.30 mmol) for 3-oxo-3,4-dihydro-2H-pyrido[3,2-b][1,4]thiazine-6-carboxylic acid: LCMS (ES) m/e 477 (M+H)⁺; ¹H NMR (CDCl₃, 400 MHz) δ 9.07 (d, J=3.3 Hz, 1H), 8.8 (br, 1H), 8.63 (s, 1H), 8.39-8.41 (m, 1H), 8.18 (d, J=9.0 Hz, 1H), 8.12 (s, 1H), 7.44-7.47 (m, 1H), 7.33-7.6 (m, 1H), 7.08 (d, J=9.0 Hz, 1H), 4.08 (s, 3H), 3.38-3.42 (m, 2H), 2.95-3.02 (m, 4H), 2.81-2.88 (m, 6H).

This material, as a solution in MeOH, was treated with an excess of 4M HCl in dioxane and evaporated to dryness to provide the dihydrochloride salt of the title compound.

### EXAMPLE 18

### Preparation of N-((2R,5S)-4-{2-[3-fluoro-6-(methyloxy)-1,5-naphthyridin-4-yl]ethyl}-2,5-dimethyl-1-piperazinyl)-3-oxo-3,4-dihydro-2H-pyrido[3,2-b][1,4]thiazine-6-carboxamide

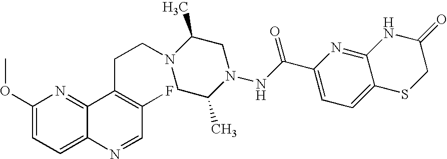

The title compound (40 mg, 53%) was prepared as a yellow solid according to Example 7, except substituting 8-ethenyl-7-fluoro-2-(methyloxy)-1,5-naphthyridine (239 mg, 1.17 mmol) for 8-ethenyl-2-(methyloxy)-1,5-naphthyridine and (2R,5S)-N-(1,1-dimethylethyl)-2,5-dimethyl-1-piperazinecarboxamide (250 mg, 1.17 mmol) for 1,1-dimethylethyl 1-piperazinecarboxylate: LCMS (ES) m/e 526 (M+H)⁺; ¹H NMR (CDCl₃, 400 MHz) δ 8.55 (s, 2H), 8.12 (d, J=9.0 Hz, 1H), 8.07 (s, 1H), 7.82 (d, J=7.8 Hz, 1H), 7.71 (d, J=7.8 Hz, 1H), 7.01 (d, J=9.0 Hz, 1H), 4.01 (s, 3H), 3.48 (s, 2H), 3.29-3.38 (m, 2H), 3.02-3.11 (m, 3H), 2.86-2.95 (m, 2H), 2.65-2.78 (m, 2H), 2.39-2.47 (m, 1H), 1.01-1.12 (m, 6H).

This material, as a solution in MeOH, was treated with an excess of 4M HCl in dioxane and evaporated to dryness to provide the dihydrochloride salt of the title compound.

### EXAMPLE 19

### Preparation of N-(4-{2-[3-fluoro-6-(methyloxy)-1,5-naphthyridin-4-yl]ethyl}-2-methyl-1-piperazinyl)-3-oxo-3,4-dihydro-2H-pyrido[3,2-b][1,4]thiazine-6-carboxamide

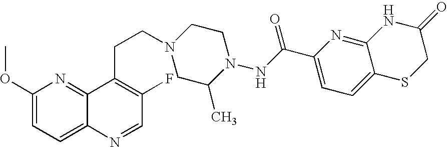

The title compound (99 mg, 57%) was prepared as a yellow solid according to Example 7, except substituting 8-ethenyl-7-fluoro-2-(methyloxy)-1,5-naphthyridine (190 mg, 0.931 mmol) for 8-ethenyl-2-(methyloxy)-1,5-naphthyridine and 2-methylpiperazine (187 mg, 1.86 mmol) for 1,1-dimethylethyl 1-piperazinecarboxylate: LCMS (ES) m/e 512 (M+H)⁺; ¹H NMR (CD₃OD, 400 MHz) δ 8.65 (s, 1H), 8.21 (d, J=9.0 Hz, 1H), 7.9 (d, J=7.8 Hz, 1H), 7.71 (d, J=7.8 Hz, 1H), 7.19 (d, J=9.0 Hz, 1H), 4.13 (s, 3H), 3.6 (s, 2H), 3.5-3.57 (m, 2H), 3.07-3.19(m, 3H), 2.81-2.97 (m, 4H), 2.59-2.64 (m,1H), 2.22-2.30 (m, 1H), 1.05 (d, J=6.4 Hz, 3H).

This material, as a solution in MeOH, was treated with an excess of 4M HCl in dioxane and evaporated to dryness to provide the dihydrochloride salt of the title compound.

### EXAMPLE 20

### Preparation of N-(4-{2-[3-fluoro-6-(methyloxy)-1,5-naphthyridin-4-]ethyl}hexahydro-1H-1,4-diazepin-1-yl)-3-oxo-3,4-dihydro-2H-pyrido[3,2-b][1,4]thiazine-6-carboxamide

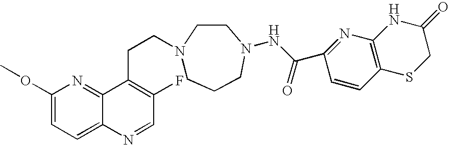

The title compound (15 mg, 23%) was prepared as a yellow solid according to Example 7, except substituting 8-ethenyl-7-fluoro-2-(methyloxy)-1,5-naphthyridine (250 mg, 1.23 mmol) for 8-ethenyl-2-(methyloxy)-1,5-naphthyridine and hexahydro-1H-1,4-diazepine (252 μl, 1.23 mmol) for 1,1-dimethylethyl 1-piperazinecarboxylate: LCMS (ES) m/e 526 (M+H)⁺; ¹H NMR (CDCl₃, 400 MHz) δ 9.31 (s, 1H), 8.56 (s,1H), 8.45 (bs, 1H), 8.15 (d, J=9.0 Hz, 1H), 7.81 (d, J=7.8 Hz, 1H), 7.69 (d, J=7.8 Hz, 1H), 7.02 (d, J=9.0 Hz, 1H), 4.00 (s, 3H), 3.41-3.46 (m, 4H), 3.25-3.29 (m, 2H), 3.12-3.17 (m, 2H), 2.95-3.02 (m, 4H), 2.88-2.92 (m, 2H), 1.82-1.86 (bs, 2H).

This material, as a solution in MeOH, was treated with an excess of 4M HCl in dioxane and evaporated to dryness to provide the dihydrochloride salt of the title compound.

### EXAMPLE 21

### Preparation of N-(4-{2-[3-fluoro-6-(methyloxy)-1,5-naphthyridin-4-yl]-2-hydroxyethyl}-1-piperazinyl)-3-oxo-3,4-dihydro-2H-pyrido[3,2-b][1,4]thiazine-6-carboxamide

### (a) 1,1-dimethylethyl 4-nitroso-1-piperazinecarboxylate

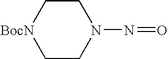

To a solution of 1,1-dimethylethyl 1-piperazinecarboxylate (5.0 g, 26 mmol) in DCM (130 mL) was added, in one portion, Diazald® (23 g, 107 mmol). After stirring at reflux for 12 h, the solution was cooled, concentrated and purified by column chromatography (silica, 5% Ethyl acetate in hexane) affording the title compound as an orange oil (4.7 g, 81%): LCMS (ES) m/e 216 (M+H)⁺.

### (b) 1,1-dimethylethyl 4-amino-1-piperazinecarboxylate

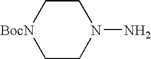

To a solution of 1,1-dimethylethyl 4-nitroso-1-piperazinecarboxylate (500 mg, 2.33 mmol) in THF (23 mL) at 0° C. was added dropwise a solution of LAH (5.8 mL, 5.8 mmol, 1M in THF). The reaction mixture warmed to 25° C. over 12 h and was subsequently quenched by dropwise addition of a saturated solution of potassium sodium tartrate. The aqueous phase was extracted several times with DCM and the combined organic fractions were dried (Na₂SO₄) and concentrated at 25° C. yielding a yellow oil that was used without further purification (270 mg, 58%): LCMS (ES) m/e 202 (M+H)⁺.

### (c) 1,1-dimethylethyl 4-{[(3-oxo-3,4-dihydro-2H-pyrido[3,2-b][1,4]thiazin-6-yl)carbonyl]amino}-1-piperazinecarboxylate

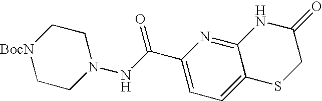

To a solution of 1,1-dimethylethyl 4-amino-1-piperazinecarboxylate (270 mg, 1.34 mmol) in DCM-DMF (4:1, 15 mL) were added 3-oxo-3,4-dihydro-2H-pyrido[3,2-b][1,4]thiazine-6-carboxylic acid (283 mg, 1.34 mmol), EDC (250 mg, 1.61 mmol) and HOBT (217 mg, 1.61 mmol). After 12 h, the solution was concentrated and the residue purified via column chromatography (silica, 1% MeOH in DCM (1% NH₄OH)) yielding the title compound as a yellow foam (412 mg, 78%): LCMS (ES) m/e 394 (M+H)⁺.

### (d) 3-oxo-N-1-piperazinyl-3,4-dihydro-2H-pyrido[3,2-b][1,4]thiazine-6-carboxamide

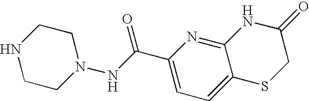

To a solution of 1,1-dimethylethyl 4-{[(3-oxo-3,4-dihydro-2H-pyrido[3,2-b][1,4]thiazin-6-yl)carbonyl]amino}-1-piperazinecarboxylate (212 mg, 0.539 mmol) in MeOH (5 mL) at 25° C. was added dropwise a solution of HCl in dioxane (809 μL, 3.24 mmol, 4M in dioxane). After 12 h, the solution was concentrated. The resulting residue was dissolved in DCM (5 mL) and neutralized with DIPEA (0.48 mL). The solution was concentrated and purified through a silica plug (5% MeOH in DCM (1% NH₄OH)) yielding the title compound as a yellow oil (278 mg, 91%): LCMS (ES) m/e 294 (M+H)⁺.

### (e) N-(4-{2-[3-fluoro-6-(methyloxy)-1,5-naphthyridin-4-yl]-2-hydroxyethyl}-1-piperazinyl)-3-oxo-3,4-dihydro-2H-pyrido[3,2-b][1,4]thiazine-6-carboxamide

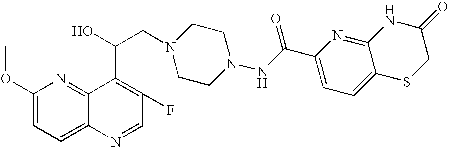

A solution of 7-fluoro-2-(methyloxy)-8-(2-oxiranyl)-1,5-naphthyridine (188 mg, 0.853 mmol) and 3-oxo-N-1-piperazinyl-3,4-dihydro-2H-pyrido[3,2-b][1,4]thiazine-6-carboxamide (250 mg, 0.853 mmol) in DMF (1 mL) was heated to 90° C. After 12 h, the solution was cooled, concentrated and the residue purified via column chromatography (silica, 0-1% MeOH in DCM (1% NH₄OH)) yielding the title compound as a yellow solid (150 mg, 31%): LCMS (ES) m/e 514 (M+H)⁺; ¹H NMR (CD₃OD, 400 MHz) δ 8.69 (s, 1H), 8.25 (d, J=9.1 Hz, 1H), 7.89 (d, J=7.9 Hz, 1H), 7.70 (d, J=7.9 Hz, 1H), 7.22 (dd, J=6.0 Hz, 1H), 4.11 (s, 3H), 3.61 (s, 2H), 3.32-3.34 (m, 1H), 2.93-3.17 (m, 1H), 2.86-2.89 (br m, 5H), 2.72-2.79 (br m, 3H).

This material underwent chiral separation using a Chiralpak AD-H column (4.6×150 mm) with MeOH (0.1% isopropylamine) as the mobile phase at a flow rate of 1 ml/min. The faster eluting enantiomer, with a retention time of 9 min, was assigned 21 E1 (GSK506782) and the slower eluting enantiomer, with a retention time of 17.3 min, was assigned 21 E2 (GSK506781). The E1 and E2 enantiomers were obtained with ee's of >99% and >94.5% respectively.

### EXAMPLE 22

### Preparation of N-(4-{2-[3-cyano-6-(methyloxy)-1,5-naphthyridin-4-yl]ethyl}-1-piperazinyl)-3-oxo-3,4-dihydro-2H-pyrido[3,2-b][1,4]thiazine-6-carboxamide

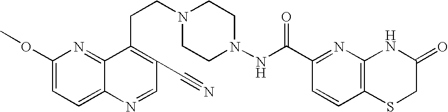

The title compound (95 mg, 39%) was prepared as a white solid according to Example 21 except substituting 4-ethenyl-6-(methyloxy)-1,5-naphthyridine-3-carbonitrile (113 mg, 0.535 mmol) for 8-ethenyl-7-fluoro-2-(methyloxy)-1,5-naphthyridine: LCMS (+ve ion electrospray) m/z 505 (MH+)¹H NMR (CHCl₃, 400 MHz) δ 8.89 (s,1H), 8.26 (d, J=9.08 Hz, 1H), 7.88 (d, J=7.90 Hz, 1H), 7.79 (d, J=7.93 Hz, 1H), 7.28 (d, J=9.01 Hz, 1H), 4.14 (s, 3H), 3.72 (br m, 2H), 3.56 (s, 2H), 3.51 (d, 1H), 3.09 (br m, 9H).

### EXAMPLE 23

### Preparation of N-[4-{2-[3-fluoro-6-(methyloxy)-1,5-naphthyridin-4-yl]ethyl}-2-(hydroxymethyl)-1-piperazinyl]-3-oxo-3,4-dihydro-2H-pyrido[3,2-b][1,4]thiazine-6-carboxamide

### (a) phenylmethyl 1,4-bis(phenylmethyl)-2-piperazinecarboxylate

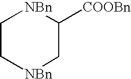

To a solution of piperazine-2-carboxylic acid dihydrochloride (4 g, 4.92 mmol) in dioxane-DMF (35 mL, 2.5:1) at 25° C. was added triethylamine (3.43 mL, 24.62 mmol) followed by benzyl chloride (25 mL, 24.62 mmol). After stirring at 100° C. for 12 h, the solution was cooled and partitioned between sat. NaCl aqueous solution and DCM. The aqueous layer was extracted several times with DCM. The combined organic fractions were dried (Na₂SO₄), concentrated and purified by column chromatography (silica, 5% ethyl acetate in hexane) affording the title compound as an off-white solid (1.2 g, 61%): LCMS (ES) m/e 401 (M+H)⁺.

### (b) [1,4-bis(phenylmethyl)-2-piperazinyl]methanol

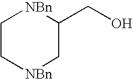

To a solution of phenylmethyl 1,4-bis(phenylmethyl)-2-piperazinecarboxylate (4 g, 1 mmol) in THF (100 mL) at 0° C. was added dropwise a solution of LAH (12 mL, 12 mmol, 1M in THF). The reaction mixture was stirred at 0° C. for 0.5 h and was subsequently quenched by dropwise addition of a saturated solution of potassium sodium tartrate. The aqueous phase was extracted several times with DCM and the combined organic fractions were dried (Na₂SO₄), concentrated and purified by column chromatography (silica, 10-50% ethyl acetate in hexane) yielding the title compound as a yellow oil (2.3 g, 74%): LCMS (ES) m/e 297 (M+H)⁺.

### (c) 2-({[(1,1-dimethylethyl)(dimethyl)silyl]oxy}methyl)-1,4-bis(phenylmethyl)piperazine

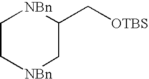

To a solution of [1,4-bis(phenylmethyl)-2-piperazinyl]methanol (1.1 g, 3.72 mmol) in DCM (37 mL) at 0° C. was added triethylamine (625 mL, 4.46 mmol), DMAP (454 mg, 3.72 mmol) and chloro(1,1-dimethylethyl)dimethylsilane (672 mg, 4.46 mmol). After stirring at 0° C. for 1 h, the solution was partitioned between water and DCM. The aqueous layer was extracted several times with DCM. The combined organic fractions were concentrated and purified through a silica plug (50% ethyl acetate in hexane) affording the title compound as an off-white solid (1.5 g, 98%): LCMS (ES) m/e 411 (M+H)⁺.

### (d) 2-({[(1,1-dimethylethyl)(dimethyl)silyl]oxy}methyl)piperazine

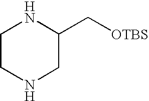

A solution of Pd(OH)₂ (30 wt %, 450 mg) and 2-({[(1,1-dimethylethyl)(dimethyl)silyl]oxy}methyl)-1,4-bis(phenylmethyl)piperazine (1.5 g, 3.66 mmol) in EtOH (36 mL) was hydrogenated at 50 psi using a Parr Shaker. After 12 h, the solution was filtered through Celite, concentrated and purified via column chromatography (silica, 5% MeOH in DCM (1% NH₄OH)) yielding the title compound as a clear oil (450 mg, 57%): LCMS (ES) m/e 211 (M+H)⁺.

### (e) 8-{2-[3-({[(1,1-dimethylethyl)(dimethyl)silyl]oxy}methyl)-1-piperazinyl]ethyl}-7-fluoro-2-(methyloxy)-1,5-naphthyridine

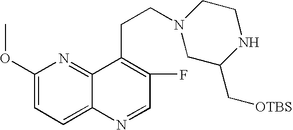

8-ethenyl-7-fluoro-2-(methyloxy)-1,5-naphthyridine (713 mg, 3.48 mmol) and 2-({[(1,1-dimethylethyl)(dimethyl)silyl]oxy}methyl)piperazine (765 mg, 3.48 mmol) were combined in EtOH (0.5 mL) and stirred at 90° C. over 12 h. The solution was then concentrated and the residue purified via column chromatography (silica, 3% MeOH in DCM (1% NH₄OH)) yielding the title compound (1 g, 66%) as an orange oil: LCMS (ES) m/e 435 (M+H)⁺.

### (f) 8-{2-[3-({[(1,1-dimethylethyl)(dimethyl)silyl]oxy}methyl)-4-nitroso-1-piperazinyl]ethyl}-7-fluoro-2-(methyloxy)-1,5-naphthyridine

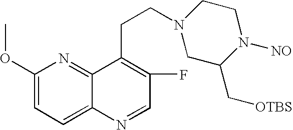

To a solution of 8-{2-[3-({[(1,1-dimethylethyl)(dimethyl)silyl]oxy}methyl)-1-piperazinyl]ethyl}-7-fluoro-2-(methyloxy)-1,5-naphthyridine (1.0 g, 2.3 mmol) in DCE (23 mL) was added, in one portion, Diazald® (2.5 g, 11.52 mmol). After stirring at reflux at 80° C. for 12 h, the solution was cooled, concentrated and purified by column chromatography (silica, 0.5% methanol in DCM) affording the title compound as an orange oil (178 mg, 17%): LCMS (ES) m/e 465 (M+H)⁺.

### (g) (1-amino-4-{2-[3-fluoro-6-(methyloxy)-1,5-naphthyridin-4-yl]ethyl}-2-piperazinyl)methanol

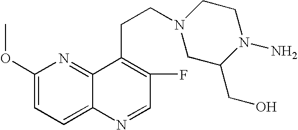

To a solution of 8-{2-[3-({[(1,1-dimethylethyl)(dimethyl)silyl]oxy}methyl)-4-nitroso-1-piperazinyl]ethyl}-7-fluoro-2-(methyloxy)-1,5-naphthyridine (178 mg, 0.38 mmol) in THF (4 mL) at 0° C. was added dropwise a solution of LAH (1.2 mL, 1.2 mmol, 1M in THF). The reaction mixture warmed to 25° C. over 12 h and was subsequently quenched by dropwise addition of a saturated solution of potassium sodium tartrate. The aqueous phase was extracted several times with DCM and the combined organic fractions were dried (Na₂SO₄) and concentrated at 25° C. yielding a yellow oil that was used without further purification (103 mg, 80%): LCMS (ES) m/e 336 (M+H)⁺.

### (h) N-[4-{2-[3-fluoro-6-(methyloxy)-1,5-naphthyridin-4-yl]ethyl}-2-(hydroxymethyl)-1-piperazinyl]-3-oxo-3,4-dihydro-2H-pyrido[3,2-b][1,4]thiazine-6-carboxamide

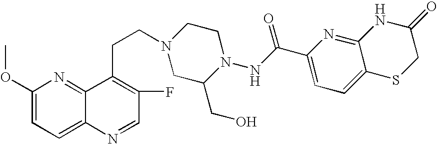

To a solution of (1-amino-4-{2-[3-fluoro-6-(methyloxy)-1,5-naphthyridin-4-yl]ethyl}-2-piperazinyl)methanol (103 mg, 0.307 mmol) in DCM-DMF (4:1, 5 mL) were added 3-oxo-3,4-dihydro-2H-pyrido[3,2-b][1,4]thiazine-6-carboxylic acid (65 mg, 0.307 mmol), EDC (57 mg, 0.369 mmol) and HOBT (50 mg, 0.369 mmol). After 12 h, the solution was concentrated and the residue purified via column chromatography (silica, 3% MeOH in DCM (1% NH₄OH)) yielding the title compound as a yellow foam (70 mg, 43%): LCMS (ES) m/e 528 (M+H)⁺; ¹H NMR (CD₃OD, 400 MHz) δ 8.55 (s, 1H), 8.11 (d, J=7.6 Hz, 1H), 7.80 (d, J=7.9 Hz, 1H), 7.72 (d, J=7.9 Hz, 1H), 7.01 (d J=7.6 Hz, 1H), 4.00-4.06 (m, 4H), 3.64-3.71 (m, 1H), 3.47 (s, 2H), 3.29-3.3.39 (m, 3H), 3.17-3.19 (m, 1H), 2.95-3.03 (m, 3H), 2.77-2.84 (m, 2H), 2.51-2.61 (m, 2H).

This material, as a solution in MeOH, was treated with an excess of 4M HCl in dioxane and evaporated to dryness to provide the dihydrochloride salt of the title compound.

### EXAMPLE 24

### Preparation of N-{4-{2-[3-fluoro-6-(methyloxy)-1,5-naphthyridin-4-yl]ethyl}-2-[(methyloxy)methyl]-1-piperazinyl}-3-oxo-3,4-dihydro-2H-pyrido[3,2-b][1,4]thiazine-6-carboxamide

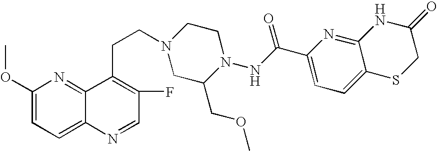

The title compound (85 mg, 57%) was prepared as a yellow foam according to Example 23, except substituting 2-[(methyloxy)methyl] (365 mg, 2.81 mmol) for 2-({[(1,1-dimethylethyl)(dimethyl)silyl]oxy}methyl)piperazine: LCMS (ES) m/e 542 (M+H)⁺; ¹H NMR (CDCl₃, 400 MHz) δ 8.55 (s, 1H), 8.11 (d, J=7.6 Hz, 1H), 7.79 (d, J=7.9 Hz, 1H), 7.70 (d, J=7.9 Hz, 1H), 7.01 (d J=7.6 Hz, 1H), 4.00 (s, 3H), 3.49-3.61 (m, 4H), 3.25-3.33 (m, 3H), 3.18 (s, 2H), 3.03-3.12 (m, 2H), 2.87-2.94 (m, 2H), 2.80-2.87 (m, 1H), 2.69-2.73 (m, 2H), 2.51-2.61 (m, 1H), 2.30-2.37 (m, 1H).

This material, as a solution in MeOH, was treated with an excess of 4M HCl in dioxane and evaporated to dryness to provide the dihydrochloride salt of the title compound.

| Example | Structure | Formula |
|---|---|---|
| 1 | 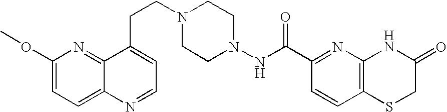 | N-(4-{2-[6-(methyloxy)-1,5- naphthyridin-4-yl]ethyl}-1- piperazinyl)-3-oxo-3,4-dihydro-2H- pyrido[3,2-b][1,4]thiazine-6- carboxamide |
| 2 | 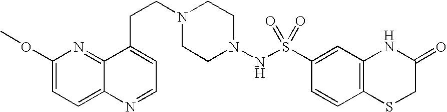 | N-(4-{2-[6-(methyloxy)-1,5- naphthyridin-4-yl]ethyl}-1- piperazinyl)-3-oxo-3,4-dihydro-2H- pyrido[3,2-b][1,4]thiazine-6- sulfonamide |
| 3 | 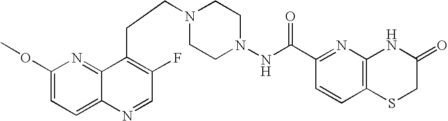 | N-(4-{2-[3-fluoro-6-(methyloxy)-1,5- naphthyridin-4-yl]ethyl}-1- piperazinyl)-3-oxo-3,4-dihydro-2H- pyrido[3,2-b][1,4]thiazine-6- carboxamide |
| 4 | 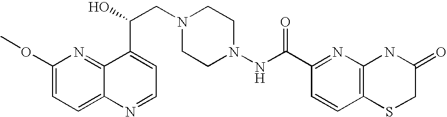 | N-(4-{(2S)-2-hydroxy-2-[6- (methyloxy)-1,5-naphthyridin-4- yl]ethyl}-1-piperazinyl)-3-oxo-3,4- dihydro-2H-pyrido[3,2- b][1,4]thiazine-6-carboxamide |
| 5 | 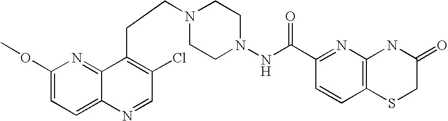 | N-(4-{2-[3-chloro-6-(methyloxy)- 1,5-naphthyridin-4-yl]ethyl}-1- piperazinyl)-3-oxo-3,4-dihydro-2H- pyrido[3,2-b][1,4]thiazine-6- carboxamide |
| 6 | 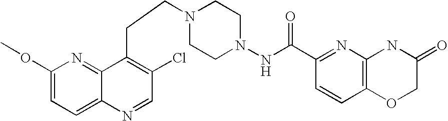 | N-(4-{2-[3-chloro-6-(methyloxy)- 1,5-naphthyridin-4-yl]ethyl}-1- piperazinyl)-3-oxo-3,4-dihydro-2H- pyrido[3,2-b][1,4]oxazine-6- carboxamide |
| 7 | 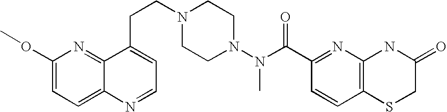 | N-methyl-N-(4-{2-[6-(methyloxy)- 1,5-naphthyridin-4-yl]ethyl}-1- piperazinyl)-3-oxo-3,4-dihydro-2H- pyrido[3,2-b][1,4]thiazine-6- carboxamide |
| 8 | 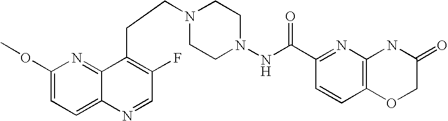 | N-4-{2-[3-fluoro-6-(methyloxy)-1,5- naphthyridin-4-yl]ethyl}-1- piperazinyl)-3-oxo-3,4-dihydro-2H- pyrido[3,2-b][1,4]oxazine-6- carboxamide |
| 9 | 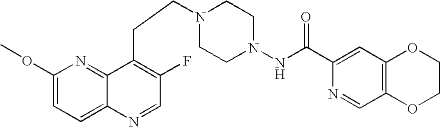 | N-(4-{2-[3-fluoro-6-(methyloxy)-1,5- naphthyridin-4-yl]ethyl}-1- piperazinyl)-2,3- dihydro[1,4]dioxino[2,3-c]pyridine- 7-carboxamide |
| 10 | 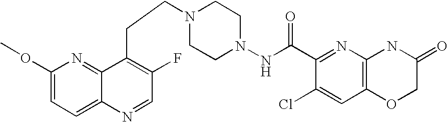 | 7-chloro-N-(4-{2-[3-fluoro-6- (methyloxy)-1,5-naphthyridin-4- yl]ethyl}-1-piperazinyl)-3-oxo-3,4- dihydro-2H-pyrido[3,2- b][1,4]oxazine-6-carboxamide |
| 11 | 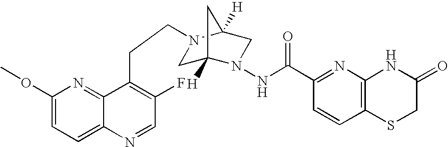 | N-((1S,4S)-5-{2-[3-fluoro-6- (methyloxy)-1,5-naphthyridin-4- yl]ethyl}-2,5- diazabicyclo[2.2.1]hept-2-yl)-3-oxo- 3,4-dihydro-2H-pyrido[3,2- b][1,4]thiazine-6-carboxamide |
| 12 | 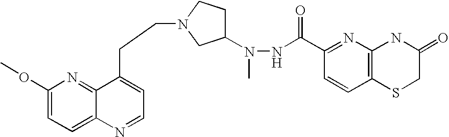 | N′-methyl-N′-(1-{2-[6-(methyloxy)- 1,5-naphthyridin-4-yl]ethyl}-3- pyrrolidinyl)-3-oxo-3,4-dihydro-2H- pyrido[3,2-b][1,4]thiazine-6- carboxamide |
| 13 | 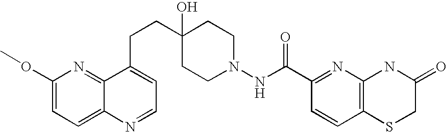 | N-(4-hydroxy-4-{2-[6-(methyloxy)- 1,5-naphthyridin-4-yl]ethyl}-1- piperidinyl)-3-oxo-3,4-dihydro-2H- pyrido[3,2-b][1,4]thiazine-6- carboxamide |
| 14 | 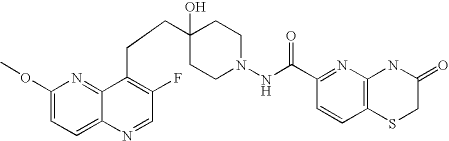 | N-(4-{2-[3-fluoro-6-(methyloxy)-1,5- naphthyridin-4-yl]ethyl}-4-hydroxy- 1-piperidinyl)-3-oxo-3,4-dihydro- 2H-pyrido[3,2-b][1,4]thiazine-6- carboxamide |
| 15 | 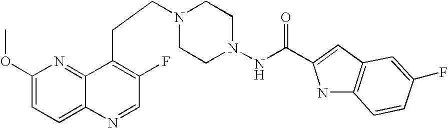 | 5-fluoro-N-(4-{2-[3-fluoro-6- (methyloxy)-1,5-naphthyridin-4- yl]ethyl}-1-piperazinyl)-1H-indole-2- carboxamide |
| 16 | 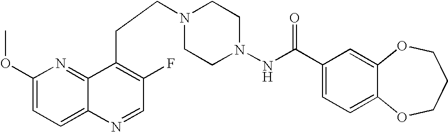 | N-(4-{2-[3-fluoro-6-(methyloxy)-1,5- naphthyridin-4-yl]ethyl}-1- piperazinyl)-3,4-dihydro-2H-1,5- benzodioxepin-7-carboxamide |
| 17 | 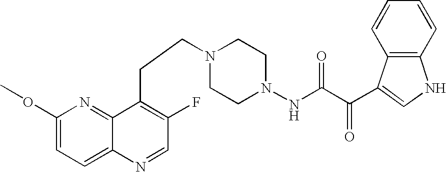 | N-(4-{2-[3-fluoro-6-(methyloxy)-1,5- naphthyridin-4-yl]ethyl}-1- piperazinyl)-2-(1H-indol-3-yl)-2- oxoacetamide |
| 18 | 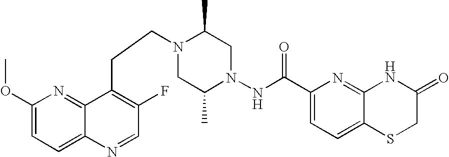 | N-((2R,5S)-4-{2-[3-fluoro-6- (methyloxy)-1,5-naphthyridin-4- yl]ethyl}-2,5-dimethyl-1- piperazinyl)-3-oxo-3,4-dihydro-2H- pyrido[3,2-b][1,4]thiazine-6- carboxamide |
| 19 | 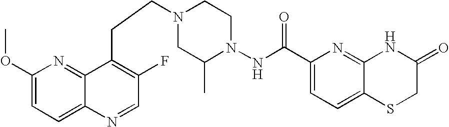 | N-(4-{2-[3-fluoro-6-(methyloxy)-1,5- naphthyridin-4-yl]ethyl}-2-methyl-1- piperazinyl)-3-oxo-3,4-dihydro-2H- pyrido[3,2-b][1,4]thiazine-6- carboxamide |
| 20 | 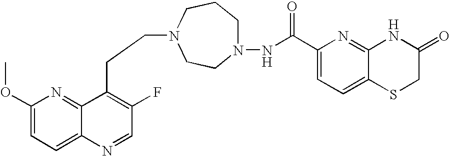 | N-(4-{2-[3-fluoro-6-(methyloxy)-1,5- naphthyridin-4-yl]ethyl}hexahydro- 1H-1,4-diazepin-1-yl)-3-oxo-3,4- dihydro-2H-pyrido[3,2- b][1,4]thiazine-6-carboxamide |
| 21 E1 | 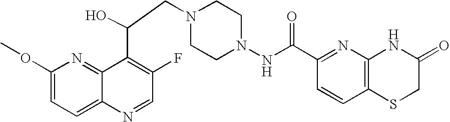 | N-(4-{2-[3-fluoro-6-(methyloxy)-1,5- naphthyridin-4-yl]-2-hydroxyethyl}- 1-piperazinyl)-3-oxo-3,4-dihydro- 2H-pyrido[3,2-b][1,4]thiazine-6- carboxamide (E1 isomer) |
| 21 E2 | 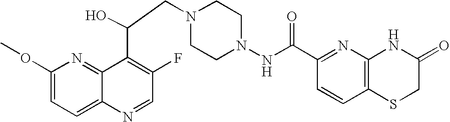 | N-(4-{2-[3-fluoro-6-(methyloxy)-1,5- naphthyridin-4-yl]-2-hydroxyethyl}- 1-piperazinyl)-3-oxo-3,4-dihydro- 2H-pyrido[3,2-b][1,4]thiazine-6- carboxamide (E2 isomer) |
| 22 | 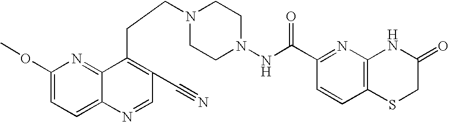 | N-(4-{2-[3-cyano-6-(methyloxy)- 1,5-naphthyridin-4-yl]ethyl}-1- piperazinyl)-3-oxo-3,4-dihydro-2H- pyrido[3,2-b][1,4]thiazine-6- carboxamide |
| 23 | 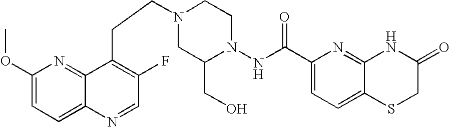 | N-[4-{2-[3-fluoro-6-(methyloxy)-1,5- naphthyridin-4-yl]ethyl}-2- (hydroxymethyl)-1-piperazinyl]-3- oxo-3,4-dihydro-2H-pyrido[3,2- b][1,4]thiazine-6-carboxamide |
| 24 | 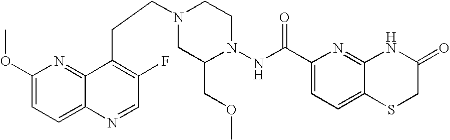 | N-{4-{2-[3-fluoro-6-(methyloxy)-1,5- naphthyridin-4-yl]ethyl}-2- [(methyloxy)methyl]-1-piperazinyl}- 3-oxo-3,4-dihydro-2H-pyrido[3,2- b][1,4]thiazine-6-carboxamide |

### EXAMPLE 25

Antimicrobial Activity Assay:

Whole-cell antimicrobial activity was determined by broth microdilution using the National Committee for Clinical Laboratory Standards (NCCLS) recommended procedure, Document M7-A6, “Methods for Dilution Susceptibility Tests for Bacteria that Grow Aerobically”. The compounds were tested in serial two-fold dilutions ranging from 0.016 to 16 mcg/mL.

Compounds were evaluated against a panel of Gram-positive organisms, including Staphylococcus aureus, Streptococcus pneumoniae, Streptococcus pyogenes, Enterococcus faecalis and Enterococcus faecium.

In addition, compounds were evaluated against a panel of Gram-negative strains including Haemophilus influenzae, Moraxella catarrhalis, Escherichia coli, Legionella Pneumophila, Chlamydophila Pneumoniae, Pseudomonas aeruginosa, Proteus mirabilis, Enterobacter cloacae, Enterobacter aerogenes, Klebsiella pneumoniae and Stenotrophomonas maltophilia.

The minimum inhibitory concentration (MIC) was determined as the lowest concentration of compound that inhibited visible growth. A mirror reader was used to assist in determining the MIC endpoint.

One skilled in the art would consider any compound with a MIC of less than 20 mg/mL to be a potential lead compound. For instance, each of the listed Examples (1 to 24, with the exception of Example 2), as identified in the present application, had a MIC ≦20 mg/ml against at least one of the organisms listed above.

### EXAMPLE 26

Rat Infection Model

Certain compounds of this invention were tested in the rat infection model. Specific pathogen-free male Sprague-Dawley CD rats were used for all bacterial strains. Each therapy group consists of 5 animals. Infection was carried out by intrabronchial instillation of 100 ml bacterial suspension for H. influenzae H128, and 50 ml of bacterial suspension for S. pneumoniae 1629 via non-surgical intubation. All compounds were administered at 1, 7, 24 and 31 hour post infection via oral gavage. In each experiment, an additional group of animals was included and served as untreated infected controls. Approximately 17 hour after the end of therapy, the animals were killed and their lungs excised and enumeration of the viable bacteria was conducted by standard methods. The lower limit of detection was 1.7 log 10 CFU/lungs.

In vivo, activity was observed in infection models in rats versus S. pneumoniae 1629 at doses ranging from 25-100 mg/Kg with oral dosing and for some compounds versus H. influenzae H128 at doses from 25-100 mg/Kg with oral dosing. Certain formula (I) compounds showed a greater than 2 log drop in viable counts in the lungs compared to non-treated controls versus S. pneumoniae 1629. Certain compounds of formula (I) showed greater than a 4 log drop in viable counts in the lungs compared to non-treated controls versus H. influenzae H 128.

It is to be understood that the invention is not limited to the embodiments illustrated hereinabove and the right is reserved to the illustrated embodiments and all modifications coming within the scope of the following claims.

1. A compound of formula (I) 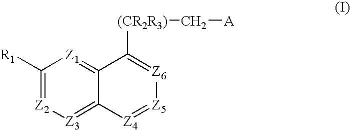
wherein:
Z₁, Z₃, and Z₄ are independently N or CR^{1a};
Z₂, Z₅, and Z₆ are each CR^{1a};
R₁ and R^{1a} are independently at each occurrence hydrogen; cyano; halogen; hydroxy; (C₁₋₆)alkoxy unsubstituted or substituted by (C₁₋₆)alkoxy, hydroxy, amino, piperidyl, guanidino or amidino any of which is unsubstituted or N-substituted by one or two (C₁₋₆)alkyl, acyl, (C₁₋₆)alkylsulphonyl, CONH₂, hydroxy, (C₁₋₆)alkylthio, heterocyclylthio, heterocyclyloxy, arylthio, aryloxy, acylthio, acyloxy or (C₁₋₆)alkylsulphonyloxy; (C₁₋₆)alkyl; (C₁₋₆)alkylthio; trifluoromethyl; trifluoromethoxy; nitro; azido; acyl; acyloxy; acylthio; (C₁₋₆)alkylsulphonyl; (C₁₋₆)alkylsulphoxide; arylsulphonyl; arylsulphoxide; or an amino, piperidyl, guanidino or amidino group unsubstituted or N-substituted by one or two (C₁₋₆)alkyl, acyl or (C₁₋₆)alkylsulphonyl groups; or R₁ and R^{1a} of Z₂ together form ethylenedioxy;
R₂ is hydrogen; halogen; hydroxy; acyloxy; or (C₁₋₆)alkoxy;
R₃ is hydrogen;
A is 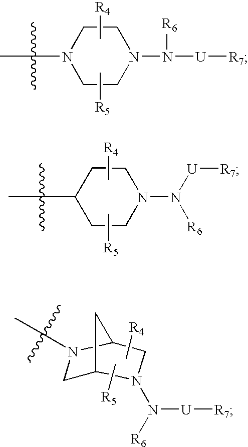 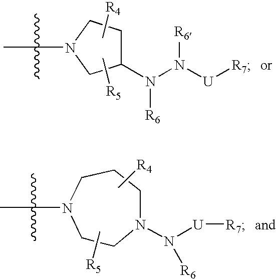
R₄ and R₅ are independently hydrogen; thiol; (C₁₋₆)alkylthio; halogen; trifluoromethyl; azido; (C₁₋₆)alkyl (optionally substituted with hydroxy or (C₁₋₆)alkoxy); (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonyl; aryl; aralkyl; aryl; heterocyclyl; heterocyclylalkyl; hydroxy; NR^{1b}R^{1b}; (C₁₋₆)alkylsulphonyl; (C₂₋₆)alkenylsulphonyl; or (C₁₋₆)aminosulphonyl wherein the amino group is optionally and independently substituted with hydrogen; (C₁₋₆)alkyl; (C₂₋₆)alkenyl; or aralkyl;
R^{1b} and R^{1b′} are independently at each occurrence hydrogen; (C₁₋₆)alkyl; aralkyl; aryl; heterocyclyl; heterocyclylalkyl; or together with the nitrogen that they are attached to form an aziridine, azetidine, pyrrolidine, piperidine or hexamethyleneimine ring (wherein said aziridine, azetidine, pyrrolidine, piperidine or hexamethyleneimine ring are optionally substituted with from 1 to 3 substituents selected from halogen, hydroxy; cyano; nitro; (C₁₋₆)alkyl; and aryl);
R₆ and R_{6′} are independently hydrogen, trifluoromethyl; (C₁₋₆)alkyl; (C₂₋₆)alkenyl; (C₁₋₆)alkoxycarbonyl; (C₁₋₆)alkylcarbonyl; (C₂₋₆)alkenyloxycarbonyl; aryl; aralkyl; (C₃₋₈)cycloalkyl; heterocyclyl; or heterocyclylalkyl;
U is (C(═O))ₙ or SO₂;
n is 1 or 2;
R₇ is a substituted or unsubstituted bicyclic carbocyclic or heterocyclic ring system (A): 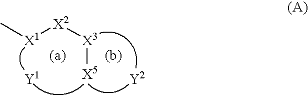
containing up to four heteroatoms in each ring in which at least one of rings (a) and (b) is aromatic;
X¹ is C or N when part of an aromatic ring or CR₈ when part of a non aromatic ring;
X² is N, NR₉, O, S(O)_{n′}, CO, a bond, or CR₈ when part of an aromatic or non-aromatic ring or may in addition be CR₁₀R₁₁ when part of a non aromatic ring;
n′ is independently at each occurrence 0, 1 or 2;
X³ and X⁵ are independently N or C;
Y is a 0 to 4 atom linker group each atom of which is independently selected from N, NR₉, O, S(O)_{n′}, CO and CR₈ when part of an aromatic or non-aromatic ring or may additionally be CR₁₀R₁₁ when part of a non aromatic ring,
Y₂ is a 2 to 6 atom linker group, each atom of Y² being independently selected from N, NR₉, O, S(O)_{n′}, CO and CR₈ when part of an aromatic or non-aromatic ring or may additionally be CR₁₀R₁₁ when part of a non aromatic ring;
R₈, R₁₀ and R₁₁ are at each occurrence independently selected from: H; (C₁₋₄)alkylthio; halogen; (C₁₋₄)alkyl; (C₂₋₄)alkenyl; hydroxy; hydroxy(C₁₋₄)alkyl; mercapto(C₁₋₄)alkyl; (C₁₋₄)alkoxy; trifluoromethoxy; nitro; cyano; carboxy; amino or aminocarbonyl unsubstituted or substituted by (C₁₋₄)alkyl;
R₉ is at each occurrence independently hydrogen; trifluoromethyl; (C₁₋₄)alkyl unsubstituted or substituted by hydroxy, carboxy, (C₁₋₄)alkoxy, (C₁₋₆)alkylthio, halogen or trifluoromethyl; (C₂₋₄)alkenyl; or aminocarbonyl wherein the amino group is optionally substituted with (C₁₋₄)alkyl;
or a pharmaceutically acceptable salt or solvate thereof.

2. A compound or salt according to claim 1, wherein
Z₁ and Z₄ are N; and
Z₃ is CR^{1a}.

3. A compound or salt according to claim 1, wherein:
R₁ is OCH₃.

4. A compound or salt according to claim 1, wherein R^{1a} is at each occurrence independently hydrogen; halogen; or cyano.

5. A compound or salt according to claim 1, wherein:
A is

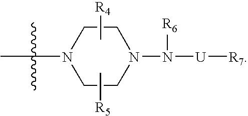

6. A compound or salt according to claim 1, wherein:
A is

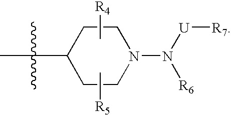

7. A compound or salt according to claim 1, wherein:
A is

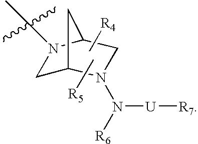

8. A compound or salt according to claim 1, wherein:
A is

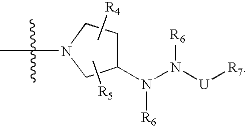

9. A compound or salt according to claim 1, wherein:
A is

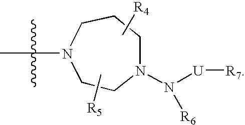

10. A compound or salt according to claim 1, wherein R₇ is:
Indol-3-yl;
5-Fluoro-indol-2-yl;
4H-Pyrido[3,2-b][1,4]thiazin-3-oxo-6-yl;
4H-Pyrido[3,2-b][1,4]oxazin-3-oxo-6-yl;
2,3-Dihydro-[1,4]dioxino[2,3-c]-pyridin-7-yl;
4H-Benzo[3,2-b][1,4]thiazin-3-oxo-6-yl;
4H-Pyrido[3,2-b][1,4]oxazin-7-chloro-3-oxo-6-yl; or
3,4-Dihydro-2H-benzo[b][1,4]dioxepin-7-yl.

11. A compound or salt according to claim 5, wherein:
R₁ is OCH₃;
Z₁ and Z₄ are N;
Z₃ is CR^{1a};
R^{1a} of Z₂, Z₃, and Z₅ is hydrogen;
R^{1a} of Z₆ is hydrogen, fluoro, chloro, or cyano;
R₂ is hydrogen or hydroxy;
R₄ and R₅ are independently hydrogen, hydroxy or (C₁₋₆)alkyl (optionally substituted with hydroxy or (C₁₋₆)alkoxy); and
R₆ is hydrogen or (C₁₋₆)alkyl.

12. A compound or salt according to claim 11, wherein R₇ is:
Indol-3-yl;
5-Fluoro-indol-2-yl;
4H-Pyrido[3,2-b][1,4]thiazin-3-oxo-6-yl;
4H-Pyrido[3,2-b][1,4]oxazin-3-oxo-6-yl;
2,3-Dihydro-[1,4]dioxino[2,3-c]-pyridin-7-yl;
4H-Benzo[3,2-b][1,4]thiazin-3-oxo-6-yl;
4H-Pyrido[3,2-b][1,4]oxazin-7-chloro-3-oxo-6-yl; or
3,4-Dihydro-2H-benzo[b][1,4]dioxepin-7-yl.

13. A compound or salt according to claim 6, wherein:
R₁ is OCH₃;
Z₁ and Z₄ are N;
Z₃ is CR^{1a};
R^{1a} of Z₂, Z₃, and Z₅ is hydrogen;
R^{1a} of Z₆ is hydrogen, fluoro, chloro, or cyano;
R₂ is hydrogen or hydroxy;
R₄ and R₅ are independently hydrogen, hydroxy or (C₁₋₆)alkyl (optionally substituted with hydroxy or (C₁₋₆)alkoxy); and
R₆ is hydrogen or (C₁₋₆)alkyl.

14. A compound or salt according to claim 13, wherein R₇ is:
Indol-3-yl;
5-Fluoro-indol-2-yl;
4H-Pyrido[3,2-b][1,4]thiazin-3-oxo-6-yl;
4H-Pyrido[3,2-b][1,4]oxazin-3-oxo-6-yl;
2,3-Dihydro[1,4]dioxino[2,3-c]-pyridin-7-yl;
4H-Benzo[3,2-b][1,4]thiazin-3-oxo-6-yl;
4H-Pyrido[3,2-b][1,4]oxazin-7-chloro-3-oxo-6-yl; or
3,4-Dihydro-2H-benzo[b][1,4]dioxepin-7-yl.

15. A compound or salt according to claim 7, wherein:
R₁ is OCH₃;
Z₁ and Z₄ are N;
Z₃ is CR^{1a};
R^{1a} of Z₂, Z₃, and Z₅ is hydrogen;
R^{1a} of Z₆ is hydrogen, fluoro, chloro, or cyano;
R₂ is hydrogen or hydroxy;
R₄ and R₅ are independently hydrogen, hydroxy or (C₁₋₆)alkyl (optionally substituted with hydroxy or (C₁₋₆)alkoxy); and
R₆ is hydrogen or (C₁₋₆)alkyl.

16. A compound or salt according to claim 15, wherein R₇ is:
Indol-3-yl;
5-Fluoro-indol-2-yl;
4H-Pyrido[3,2-b][1,4]thiazin-3-oxo-6-yl;
4H-Pyrido[3,2-b][1,4]oxazin-3-oxo-6-yl;
2,3-Dihydro-[1,4]dioxino[2,3-c]-pyridin-7-yl;
4H-Benzo[3,2-b][1,4]thiazin-3-oxo-6-yl;
4H-Pyrido[3,2-b][1,4]oxazin-7-chloro-3-oxo-6-yl; or
3,4-Dihydro-2H-benzo[b][1,4]dioxepin-7-yl.

17. A compound or salt according to claim 8, wherein:
R₁ is OCH₃;
Z₁ and Z₄ are N;
Z₃ is CR^{1a};
R^{1a} of Z₂, Z₃, and Z₅ is hydrogen;
R^{1a} of Z₆ is hydrogen, fluoro, chloro, or cyano;
R₂ is hydrogen or hydroxy;
R₄ and R₅ are independently hydrogen, hydroxy or (C₁₋₆)alkyl (optionally substituted with hydroxy or (C₁₋₆)alkoxy); and
R₆ and R_{6′} are independently hydrogen or (C₁₋₆)alkyl.

18. A compound or salt according to claim 17, wherein R₇ is:
Indol-3-yl;
5-Fluoro-indol-2-yl;
4H-Pyrido[3,2-b][1,4]thiazin-3-oxo-6-yl;
4H-Pyrido[3,2-b][1,4]oxazin-3-oxo-6-yl;
2,3-Dihydro-[1,4]dioxino[2,3-c]-pyridin-7-yl;
4H-Benzo[3,2-b][1,4]thiazin-3-oxo-6-yl;
4H-Pyrido[3,2-b][1,4]oxazin-7-chloro-3-oxo-6-yl; or
3,4-Dihydro-2H-benzo[b][1,4]dioxepin-7-yl.

19. A compound or salt according to claim 9, wherein:
R₁ is OCH₃;
Z₁ and Z₄ are N;
Z₃ is CR^{1a};
R^{1a} of Z₂, Z₃, and Z₅ is hydrogen;
R^{1a} of Z₆ is hydrogen, fluoro, chloro, or cyano;
R₂ is hydrogen or hydroxy;
R₄ and R₅ are independently hydrogen, hydroxy or (C₁₋₆)alkyl (optionally substituted with hydroxy or (C₁₋₆)alkoxy); and
R₆ is hydrogen or (C₁₋₆)alkyl.

20. A compound or salt according to claim 19, wherein R₇ is:
Indol-3-yl;
5-Fluoro-indol-2-yl;
4H-Pyrido[3,2-b][1,4]thiazin-3-oxo-6-yl;
4H-Pyrido[3,2-b][1,4]oxazin-3-oxo-6-yl;
2,3-Dihydro-[1,4]dioxino[2,3-c]-pyridin-7-yl;
4H-Benzo[3,2-b][1,4]thiazin-3-oxo-6-yl;
4H-Pyrido[3,2-b][1,4]oxazin-7-chloro-3-oxo-6-yl; or
3,4-Dihydro-2H-benzo[b][1,4]dioxepin-7-yl.

21. A compound according to claim 1, wherein the compound is:
a) N-(4-{2-[6-(methyloxy)-1,5-naphthyridin-4-yl]ethyl}-1-piperazinyl)-3-oxo-3,4-dihydro-2H-pyrido[3,2-b][1,4]thiazine-6-carboxamide;
b) N-(4-{2-[3-fluoro-6-(methyloxy)-1,5-naphthyridin-4-yl]ethyl}-1-piperazinyl)-3-oxo-3,4-dihydro-2H-pyrido[3,2-b][1,4]thiazine-6-carboxamide;
c) N-(4-{(2S)-2-hydroxy-2-[6-(methyloxy)-1,5-naphthyridin-4-yl]ethyl}-1-piperazinyl)-3-oxo-3,4-dihydro-2H-pyrido[3,2-b][1,4]thiazine-6-carboxamide;
d) N-(4-{2-[3-chloro-6-(methyloxy)-1,5-naphthyridin-4-yl]ethyl}-1-piperazinyl)-3-oxo-3,4-dihydro-2H-pyrido[3,2-b][1,4]thiazine-6-carboxamide;
e) N-(4-{2-[3-chloro-6-(methyloxy)-1,5-naphthyridin-4-yl]ethyl}-1-piperazinyl)-3-oxo-3,4-dihydro-2H-pyrido[3,2-b][1,4]oxazine-6-carboxamide;
f) N-methyl-N-(4-{2-[6-(methyloxy)-1,5-naphthyridin-4-yl]ethyl}-1-piperazinyl)-3-oxo-3,4-dihydro-2H-pyrido[3,2-b][1,4]thiazine-6-carboxamide;
g) N-4-{2-[3-fluoro-6-(methyloxy)-1,5-naphthyridin-4-yl]ethyl}-1-piperazinyl)-3-oxo-3,4-dihydro-2H-pyrido[3,2-b][1,4]oxazine-6-carboxamide;
h) N-(4-{2-[3-fluoro-6-(methyloxy)-1,5-naphthyridin-4-yl]ethyl}-1-piperazinyl)-2,3-dihydro[1,4]dioxino[2,3-c]pyridine-7-carboxamide;
i) 7-chloro-N-(4-{2-[3-fluoro-6-(methyloxy)-1,5-naphthyridin-4-yl]ethyl}-1-piperazinyl)-3-oxo-3,4-dihydro-2H-pyrido[3,2-b][1,4]oxazine-6-carboxamide;
j) N-((1S,4S)-5-{2-[3-fluoro-6-(methyloxy)-1,5-naphthyridin-4-yl]ethyl}-2,5-diazabicyclo[2.2.1]hept-2-yl)-3-oxo-3,4-dihydro-2H-pyrido[3,2-b][1,4]thiazine-6-carboxamide;
k) N′-methyl-N′-(1-{2-[6-(methyloxy)-1,5-naphthyridin-4-yl]ethyl}-3-pyrrolidinyl)-3-oxo-3,4-dihydro-2H-pyrido[3,2-b][1,4]thiazine-6-carboxamide;
l) N-(4-hydroxy-4-{2-[6-(methyloxy)-1,5-naphthyridin-4-yl]ethyl}-1-piperidinyl)-3-oxo-3,4-dihydro-2H-pyrido[3,2-b][1,4]thiazine-6-carboxamide;
m) N-(4-{2-[3-fluoro-6-(methyloxy)-1,5-naphthyridin-4-yl]ethyl}-4-hydroxy-1-piperidinyl)-3-oxo-3,4-dihydro-2H-pyrido[3,2-b][1,4]thiazine-6-carboxamide;
n) 5-fluoro-N-(4-{2-[3-fluoro-6-(methyloxy)-1,5-naphthyridin-4-yl]ethyl}-1-piperazinyl)-1H-indole-2-carboxamide;
o) N-(4-{2-[3-fluoro-6-(methyloxy)-1,5-naphthyridin-4-yl]ethyl}-1-piperazinyl)-3,4-dihydro-2H-1,5-benzodioxepin-7-carboxamide;
p) N-(4-{2-[3-fluoro-6-(methyloxy)-1,5-naphthyridin-4-yl]ethyl}-1-piperazinyl)-2-(1H-indol-3-yl)-2-oxoacetamide;
q) N-((2R,5S)-4-{2-[3-fluoro-6-(methyloxy)-1,5-naphthyridin-4-yl]ethyl}-2,5-dimethyl-1-piperazinyl)-3-oxo-3,4-dihydro-2H-pyrido[3,2-b][1,4]thiazine-6-carboxamide;
r) N-(4-{2-[3-fluoro-6-(methyloxy)-1,5-naphthyridin-4-yl]ethyl}-2-methyl-1-piperazinyl)-3-oxo-3,4-dihydro-2H-pyrido[3,2-b][1,4]thiazine-6-carboxamide;
s) N-(4-{2-[3-fluoro-6-(methyloxy)-1,5-naphthyridin-4-yl]ethyl}hexahydro-1H-1,4-diazepin-1-yl)-3-oxo-3,4-dihydro-2H-pyrido[3,2-b][1,4]thiazine-6-carboxamide;
t) N-(4-{(2S)-[3-fluoro-6-(methyloxy)-1,5-naphthyridin-4-yl]-2-hydroxyethyl}-1-piperazinyl)-3-oxo-3,4-dihydro-2H-pyrido[3,2-b][1,4]thiazine-6-carboxamide;
u) N-(4-{(2R)-[3-fluoro-6-(methyloxy)-1,5-naphthyridin-4-yl]-2-hydroxyethyl}-1-piperazinyl)-3-oxo-3,4-dihydro-2H-pyrido[3,2-b][1,4]thiazine-6-carboxamide;
v) N-(4-{2-[3-cyano-6-(methyloxy)-1,5-naphthyridin-4-yl]ethyl}-1-piperazinyl)-3-oxo-3,4-dihydro-2H-pyrido[3,2-b][1,4]thiazine-6-carboxamide;
w) N-[4-{2-[3-fluoro-6-(methyloxy)-1,5-naphthyridin-4-yl]ethyl}-2-(hydroxymethyl)-1-piperazinyl]-3-oxo-3,4-dihydro-2H-pyrido[3,2-b][1,4]thiazine-6-carboxamide; or
x) N-{4-{2-[3-fluoro-6-(methyloxy)-1,5-naphthyridin-4-yl]ethyl}-2-[(methyloxy)methyl]-1-piperazinyl}-3-oxo-3,4-dihydro-2H-pyrido[3,2-b][1,4]thiazine-6-carboxamide; or
a pharmaceutically acceptable salt thereof.

22. (canceled)

23. A pharmaceutical composition comprising a compound or salt according to claim 1 and a pharmaceutically acceptable carrier.

24. A method of treating bacterial infections in mammals which comprises administering to a mammal in need thereof an effective amount of a compound or salt according to claim 1.
